SEQUENCE LISTING (54 sequences)

SEQ ID NO: 1 - RNA, 30 nt, Artificial Sequence
  RNA oligonucleotide
cucuucucucuuaaaauggcugccugucug

SEQ ID NO: 2 - DNA, 30 bp, Artificial Sequence
  DNA oligonucleotide
gagaagagagaattttaccgacggacagac

SEQ ID NO: 3 - RNA, 30 nt, Artificial Sequence
  RNA oligonucleotide
cccgccuccccaggccuacgcgcugagcuc

SEQ ID NO: 4 - DNA, 30 bp, Artificial Sequence
  DNA oligonucleotide
ctcttctctcttaaaatggctgcctgtctg

SEQ ID NO: 5 - RNA, 23 nt, Mus musculus
ggugaauuucuuccuggcagauc

SEQ ID NO: 6 - RNA, 20 nt, Photinus pyralis
ggacaucacuuacgcugagu

SEQ ID NO: 7 - DNA, 33 bp, Artificial Sequence
  Sequence used for trimming GRO-seq reads
tggaattctcgggtgccaaggaactccagtcac

SEQ ID NO: 8 - DNA, 21 bp, Artificial Sequence
  DNA adapter with 5rApp on 5′ end and 3ddC on 3′ end
tggaattctcgggtgccaagg

SEQ ID NO: 9 - DNA, 65 bp, Artificial Sequence
  DNA oligonucleotide with Sp-C18 (hexaethyleneglycol) after GAGTGT and after CACTCA
  misc_feature (2)..(5): n is A, C, G, or T
annnnagatcggaagagcgtcgtgtagggaaagagtgtcactcaccttggcacccgagaattcca

SEQ ID NO: 10 - DNA, 53 bp, Artificial Sequence
  DNA oligonucleotide
aatgatacggcgaccaccgagatctacactctttccctacacgacgctcttcc

SEQ ID NO: 11 - DNA, 55 bp, Artificial Sequence
  DNA oligonucleotide
caagcagaagacggcatacgagatcgtgatcggtccttggcacccgagaattcca

SEQ ID NO: 12 - DNA, 55 bp, Artificial Sequence
  DNA oligonucleotide
caagcagaagacggcatacgagatacatcgcggtccttggcacccgagaattcca

SEQ ID NO: 13 - DNA, 18 bp, Artificial Sequence
  DNA oligonucleotide
aatgatacggcgaccacc

SEQ ID NO: 14 - DNA, 19 bp, Artificial Sequence
  DNA oligonucleotide
caagcagaagacggcatac

SEQ ID NO: 15 - DNA, 30 bp, Artificial Sequence
  DNA oligonucleotide
ctctttccctacacgacgctcttccgatct

SEQ ID NO: 16 - DNA, 25 bp, Artificial Sequence
  DNA oligonucleotide
tggaattctcgggtgccaaggaccg

SEQ ID NO: 17 - DNA, 30 bp, Artificial Sequence
  DNA oligonucleotide
tcgctccccggccatcttggcggctggtgt

SEQ ID NO: 18 - DNA, 30 bp, Artificial Sequence
  DNA oligonucleotide
acaccagccgccaagatggccggggagcga

SEQ ID NO: 19 - DNA, 30 bp, Artificial Sequence
  DNA oligonucleotide
aggcagtggctagctcacgtccctggatcg

SEQ ID NO: 20 - DNA, 30 bp, Artificial Sequence
  DNA oligonucleotide
cgatccagggacgtgagctagccactgcct

SEQ ID NO: 21 - DNA, 30 bp, Artificial Sequence
  DNA oligonucleotide
cagacaggcagccattttaagagagaagag

SEQ ID NO: 22 - DNA, 30 bp, Artificial Sequence
  DNA oligonucleotide
cccgcctccccaggcctacgcgctgagctc

SEQ ID NO: 23 - DNA, 30 bp, Artificial Sequence
  DNA oligonucleotide
gagctcagcgcgtaggcctggggaggcggg

SEQ ID NO: 24 - DNA, 40 bp, Artificial Sequence
  DNA oligonucleotide
ggtgggagggagactgccctttgtcatgtagaaggagctt

SEQ ID NO: 25 - DNA, 40 bp, Artificial Sequence
  DNA oligonucleotide
aagctccttctacatgacaaagggcagtctccctcccacc

SEQ ID NO: 26 - DNA, 40 bp, Artificial Sequence
  DNA oligonucleotide
acgtgcagggagactgccctttgtcatgtagaagcagttg

SEQ ID NO: 27 - DNA, 40 bp, Artificial Sequence
  DNA oligonucleotide
caactgcttctacatgacaaagggcagtctccctgcacgt

SEQ ID NO: 28 - RNA, 30 nt, Artificial Sequence
  RNA oligonucleotide
cagacaggcagccauuuuaagagagaagag

SEQ ID NO: 29 - DNA, 60 bp, Artificial Sequence
  DNA/RNA chimeric oligonucleotide (first 30 nucleotides are DNA, second 30 nucleotides are RNA)
tcgctccccggccatcttggcggctggtgtcucuucucucuuaaaauggcugccugucug

SEQ ID NO: 30 - DNA, 60 bp, Artificial Sequence
  DNA/RNA chimeric oligonucleotide (first 30 nucleotides are DNA, second 30 nucleotides are RNA)
acaccagccgccaagatggccggggagcgacucuucucucuuaaaauggcugccugucug

SEQ ID NO: 31 - DNA, 20 bp, Mus musculus
gtactggctgctcaaatgtc

SEQ ID NO: 32 - DNA, 21 bp, Mus musculus
gtgatgtaggtataattagcc

SEQ ID NO: 33 - DNA, 21 bp, Mus musculus
ttgcatgttgttcctcggagc

SEQ ID NO: 34 - DNA, 21 bp, Mus musculus
ccatgtatggttacggggatc

SEQ ID NO: 35 - DNA, 20 bp, Mus musculus
gctgaaagaccacagctccc

SEQ ID NO: 36 - DNA, 21 bp, Mus musculus
ttgggtgttgagaataggtcc

SEQ ID NO: 37 - DNA, 21 bp, Artificial Sequence
  DNA oligonucleotide
ggcaaactttcggttcagtgg

SEQ ID NO: 38 - DNA, 20 bp, Artificial Sequence
  DNA oligonucleotide
ttttcctcccccaagacagg

SEQ ID NO: 39 - DNA, 23 bp, Artificial Sequence
  DNA oligonucleotide
cccaccttgtgttcaaatgctga

SEQ ID NO: 40 - DNA, 22 bp, Artificial Sequence
  DNA oligonucleotide
acgcttttcttctgccttctgc

SEQ ID NO: 41 - DNA, 21 bp, Artificial Sequence
  DNA oligonucleotide
aaccatcaggaaggcatcagg

SEQ ID NO: 42 - DNA, 21 bp, Artificial Sequence
  DNA oligonucleotide
agctgataggcactctgggta

SEQ ID NO: 43 - DNA, 21 bp, Artificial Sequence
  DNA oligonucleotide
gatctccgtacaagcaggagg

SEQ ID NO: 44 - DNA, 21 bp, Artificial Sequence
  DNA oligonucleotide
actcacctaaatctgcctgcc

SEQ ID NO: 45 - DNA, 21 bp, Artificial Sequence
  DNA oligonucleotide
agatgccagaggtcacctttg

SEQ ID NO: 46 - DNA, 21 bp, Artificial Sequence
  DNA oligonucleotide
caagaggatttgtggggctct

SEQ ID NO: 47 - DNA, 21 bp, Artificial Sequence
  DNA oligonucleotide
tacacgtgtgcgccagaagag

SEQ ID NO: 48 - DNA, 21 bp, Artificial Sequence
  DNA oligonucleotide
tctaggagcccagcccctttt

SEQ ID NO: 49 - DNA, 21 bp, Artificial Sequence
  DNA oligonucleotide
ctccattgtcctagggatgcc

SEQ ID NO: 50 - DNA, 21 bp, Artificial Sequence
  DNA oligonucleotide
actttattcacggggctccag

SEQ ID NO: 51 - DNA, 21 bp, Artificial Sequence
  DNA oligonucleotide
actgacttcctccaaggccac

SEQ ID NO: 52 - DNA, 21 bp, Artificial Sequence
  DNA oligonucleotide
tgaatgcttggtccccagtgt

SEQ ID NO: 53 - protein, 9 aa, Homo sapiens
  MISC_FEATURE (1)..(9): X is any amino acid
LXXLXXLXL

SEQ ID NO: 54 - protein, 8 aa, Homo sapiens
  MISC_FEATURE (2)..(2): X is V or I
  MISC_FEATURE (4)..(4): X is any amino acid
DXEXNPGP

(19) United States
(12) Patent Application Publication
(10) Pub. No.: US 2019/0062752 A1
(43) Pub. Date: Feb. 28, 2019

(54) Transcription Factor Trapping by RNA in Gene Regulatory Elements

(71) Applicant: Whitehead Institute for Biomedical Research, Cambridge, MA (US)

(72) Inventors: Richard A. Young, Boston, MA (US); Alla A. Sigova, Newton, MA (US); Brian J. Abraham, Cambridge, MA (US)

(21) Appl. No.: 15/771,913

(22) PCT Filed: Oct. 28, 2016
(86) PCT No.: PCT/US2016/059399

§ 371 Date: Apr. 27, 2018

Related U.S. Application Data
(60) Provisional application No. 62/248,119, filed on Oct. 29, 2015.
(60) Provisional application No. 62/266,805, filed on Dec. 14, 2015.

(51) Int. Cl. C12N 15/113

(52) U.S. Cl. CPC C12N 15/1135; C12N 2310/13

(57) ABSTRACT

Disclosed herein are methods useful for modulating expression of a target gene by modulating binding between a ribonucleic acid (RNA) transcribed from at least one regulatory element of a target gene and a transcription factor which binds to both the RNA and the regulatory element. Also disclosed herein are methods and assays for identifying agents that interfere with binding between RNA transcribed from at least one regulatory element and a transcription factor which binds to the RNA and to the regulatory element.

### CROSS-REFERENCE TO RELATED APPLICATIONS

This application claims the benefit of U.S. Provisional Application Nos. 62/248,119, filed Oct. 29, 2015, and 62/266,805, filed Dec. 14, 2015, each of which is incorporated herein by reference in its entirety.

### FEDERALLY SPONSORED RESEARCH OR DEVELOPMENT

This invention was made with government support under HG002668 awarded by the National Institutes of Health (NIH). The government has certain rights in the invention.

### INCORPORATION-BY-REFERENCE OF MATERIAL SUBMITTED ELECTRONICALLY

This application contains a sequence listing. It has been submitted electronically via EFS-Web as an ASCII text file entitled “151262-00005_ST25.txt”. The sequence listing is 10,952 bytes in size, and was created on Oct. 28, 2016. It is hereby incorporated by reference in its entirety.

### BACKGROUND

Transcription factors (TFs) bind specific sequences in promoter-proximal and distal DNA elements in order to regulate gene transcription. Active promoters and enhancer elements are transcribed bi-directionally (see e.g., Core et al., 2008; Seila et al., 2008; and Sigova et al., 2013). Although various models have been proposed for the roles of RNA species produced from these regulatory elements, their functions are not fully understood (Kim et al., 2010; Wang et al., 2011; Melo et al., Mol Cell 49, 524-535 (2013); Lai et al., 2013; Lam et al., 2013; Li et al., 2013; Kaikkonen et al., 2013; Mousavi et al., 2013; Di Ruscio et al., 2013; and Schaukowitch et al., 2014).

### SUMMARY

In one aspect, the presently disclosed subject matter provides a method of modulating expression of a target gene, the method comprising modulating binding between a ribonucleic acid (RNA) transcribed from at least one regulatory element of a target gene and a transcription factor which binds to both the RNA and the at least one regulatory element, wherein modulating binding between the RNA and the transcription factor modulates expression of the target gene. In some embodiments, the RNA is a non-coding RNA selected from the group consisting of enhancer RNA, promoter RNA, super-enhancer constituent RNA, and combinations thereof. In some embodiments, at least one regulatory element is selected from the group consisting of an enhancer, a promoter, a super-enhancer constituent, and combinations thereof.

In some embodiments, modulating binding comprises promoting binding between the RNA and the transcription factor. In some embodiments, promoting binding between the RNA and the transcription factor stabilizes occupancy of the transcription factor at the at least one regulatory element, thereby increasing expression of the target gene. In some embodiments, promoting binding between the RNA and the transcription factor comprises tethering an RNA that binds to the transcription factor to a DNA sequence in proximity to the at least one regulatory element.

In some embodiments, modulating binding comprises interfering with binding between the RNA and the transcription factor. In some embodiments, interfering with binding between the RNA and the transcription factor destabilizes occupancy of the transcription factor at the at least one regulatory element, thereby decreasing expression of the target gene.

In some embodiments, the transcription factor comprises an N-terminal region and a C-terminal region, wherein the N-terminal region binds to either the RNA or the at least one regulatory element, and the C-terminal region binds to the RNA or the at least one regulatory element which is not bound to the N-terminal region. In some embodiments, either the N-terminal region or the C-terminal region comprises a DNA binding domain selected from the group consisting of a zinc finger, leucine zipper, helix-turn-helix, winged helix-turn-helix, helix-loop-helix, HMG-box, and OB-fold. In some embodiments, either the N-terminal region or the C-terminal region comprises an RNA binding domain. In some embodiments, the transcription factor is selected from the group consisting of Yin-Yang 1 (YY1), Krueppel-like factor 4 (KLF4), Ronin (Thap11), RE1-silencing transcription factor (REST), PR domain zinc finger protein 14 (PRDM14), CCCTC-binding factor (CTCF), Signal transducer and activator of transcription 1 (STAT1), TLS/FUS, BRCA1, DLX2, ESR1, KIN, KU, NACA, NCL, NFKB1, NFYA, NR3C1, RARA, RUNX1, SOX2, TCF7, and TP53 (p53). In some embodiments, the transcription factor is Yin-Yang 1 (YY1).

In some embodiments, modulating expression of the target gene occurs in vitro or ex vivo. In some embodiments, modulating expression of the target gene comprises contacting a cell with an effective amount of an agent which interferes with binding between the RNA and the transcription factor.

In some embodiments, modulating expression of the target gene occurs in vivo. In some embodiments, modulating expression of the target gene comprises administering to a subject an effective amount of a composition which interferes with binding between the RNA and the transcription factor. In some embodiments, the composition comprises an agent which binds to the transcription factor in a manner that prevents the transcription factor from binding to the RNA. In some embodiments, the agent does not compete with a DNA sequence in the at least one regulatory element for binding to the transcription factor. In some embodiments, the agent is selected from the group consisting of small molecules, saccharides, peptides, proteins, peptidomimetics, nucleic acids, an extract made from biological materials selected from the group consisting of bacteria, plants, fungi, animal cells, and animal tissues, and any combination thereof.

In some embodiments, the agent comprises a decoy RNA. In some embodiments, the decoy RNA comprises a synthetic RNA selected from the group consisting of: (i) a synthetic RNA having a nucleotide sequence that is homologous to the RNA transcribed from the at least one regulatory element; (ii) a synthetic RNA having a nucleotide sequence that is homologous to an RNA binding site for the transcription factor; (iii) a synthetic RNA that binds to the transcription factor at a site other than the DNA binding domain of the transcription factor; (iv) a synthetic RNA having a nucleotide sequence that is at least partially complementary to the RNA transcribed from the at least one regulatory element; and (v) a synthetic RNA having a nucleotide sequence that is at least partially complementary to a binding site for the transcription factor in the RNA transcribed from the at least one regulatory element. In some embodiments, the synthetic RNA comprises a nucleotide sequence that comprises an RNA binding site for the transcription factor. In some embodiments, the synthetic RNA comprises a length of between 10 nucleotides and 300 nucleotides. In some embodiments, the synthetic RNA comprises a length of between 30 and 60 nucleotides.

In some embodiments, the synthetic RNA contains at least one modification.

In some embodiments, the composition comprises an agent which binds to the RNA in a manner that prevents the transcription factor from binding to the RNA. In some embodiments, the agent is selected from the group consisting of small molecules, saccharides, peptides, proteins, peptidomimetics, nucleic acids, an extract made from biological materials selected from the group consisting of bacteria, plants, fungi, animal cells, and animal tissues, and any combination thereof. In some embodiments, the agent is an RNA interfering agent selected from the group consisting of a ribozyme, guide RNA, small interfering RNA (siRNA), short hairpin RNA or small hairpin RNA (shRNA), microRNA (miRNA), post-transcriptional gene silencing RNA (ptgsRNA), short interfering oligonucleotide, antisense oligonucleotide, aptamer, and CRISPR RNA.

In some embodiments, the composition modifies at least one nucleotide of a DNA sequence of the at least one regulatory element in a manner that prevents RNA transcribed from the at least one regulatory element from binding to the transcription factor. In some embodiments, the composition comprises a genomic editing system selected from the group consisting of a CRISPR\Cas system, zinc finger nucleases (ZFNs), Transcription Activator-Like Effector Nucleases (TALENs), and engineered meganuclease re-engineered homing endonucleases.

In some embodiments, the composition comprises an agent which prevents exosomal degradation of untethered RNA in proximity to the at least one regulatory element or the transcriptional machinery. In some embodiments, the agent inhibits a component of the exosome. In some embodiments, the agent inhibits a component of the exosome via RNA interference.

In some embodiments, the target gene comprises a gene for which increased or aberrant transcription is associated with a disease, condition, or disorder. In some embodiments, the disease, condition, or disorder is selected from the group consisting of a cancer, a genetic disorder, a liver disorder, a neurodegenerative disorder, and an autoimmune disease. In some embodiments, the target gene comprises an oncogene. In some embodiments, the target gene comprises at least one mutation in the at least one regulatory element, wherein the at least one mutation results in the transcription factor binding to RNA transcribed from the at least one regulatory element in a manner that stabilizes occupancy of the transcription factor to the at least one regulatory element, thereby increasing expression of the target gene. In some embodiments, the at least one mutation comprises a single nucleotide polymorphism.

In some aspects, the presently disclosed subject matter provides a method of identifying a candidate agent that interferes with binding between RNA transcribed from at least one regulatory element and a transcription factor which binds to the RNA and to the at least one regulatory element, the method comprising assessing binding between RNA transcribed from at least one regulatory element and a transcription factor which binds to the RNA and to the at least one regulatory element in the presence and absence of a test agent, wherein decreased binding of the transcription factor to the RNA transcribed from the at least one regulatory element in the presence of the test agent as compared to the absence of the test agent indicates that the test agent is a candidate agent that interferes with binding between the RNA and the transcription factor.

In some embodiments, the methods further comprise identifying a transcription factor that binds to RNA transcribed from at least one regulatory element and to the at least one regulatory element. In some embodiments, the methods further comprise identifying an RNA binding domain of the transcription factor. In some embodiments, the methods further comprise identifying a consensus motif in the RNA transcribed from the at least one regulatory sequence for the RNA binding domain of the transcription factor.

In some embodiments, assessing binding comprises contacting a complex or mixture comprising the transcription factor, the at least one regulatory element, and the RNA transcribed from the at least one regulatory element with the test agent. In some embodiments, the methods further comprise assessing whether the test agent is capable of binding to the transcription factor at a site other than a DNA binding domain of the transcription factor. In some embodiments, the test agent is selected from the group consisting of small molecules, saccharides, peptides, proteins, peptidomimetics, nucleic acids, an extract made from biological materials selected from the group consisting of bacteria, plants, fungi, animal cells, and animal tissues, and any combination thereof.

In some embodiments, the test agent comprises a decoy RNA. In some embodiments, the decoy RNA comprises a synthetic RNA selected from the group consisting of: (i) a synthetic RNA having a nucleotide sequence that is homologous to the RNA transcribed from the at least one regulatory element; (ii) a synthetic RNA having a nucleotide sequence that is homologous to an RNA binding site for the transcription factor; (iii) a synthetic RNA that binds to the transcription factor at a site other than the DNA binding domain of the transcription factor; (iv) a synthetic RNA having a nucleotide sequence that is at least partially complementary to the RNA transcribed from the at least one regulatory element; and (v) a synthetic RNA having a nucleotide sequence that is at least partially complementary to a binding site for the transcription factor in the RNA transcribed from the at least one regulatory element. In some embodiments, the synthetic RNA comprises a nucleotide sequence that comprises an RNA binding site for the transcription factor. In some embodiments, the synthetic RNA comprises a length of between 10 nucleotides and 300 nucleotides. In some embodiments, the synthetic RNA comprises a length of between 30 and 60 nucleotides. In some embodiments, binding is performed in a cell. In some embodiments, the methods comprise performing cross-linking immunoprecipitation (CLIP) with the RNA and the transcription factor.

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning. A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange 10^{th} ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, available on the World Wide Web: http://www.ncbi.nlm.nih.gov/omim/ and in Online Mendelian Inheritance in Animals (OMIA), a database of genes, inherited disorders and traits in animal species (other than human and mouse), available on the World Wide Web: http://omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1A, FIG. 1B, FIG. 1C, and FIG. 1D show that YY1 binds to DNA and RNA at transcriptional regulatory elements. FIG. 1A shows a cartoon depicting divergent transcription at enhancers and promoters in mammalian cells. FIG. 1B shows an alignment of GRO-seq reads at all enhancers and promoters in ESCs. Enhancers were defined as in (Whyte et al., 2013). The x-axis indicates distance from either the enhancer center (C) or the transcription start site (TSS) in kilobases. The y-axis indicates average density of uniquely mapped GRO-seq reads per genomic bin. FIG. 1C shows gene tracks for the Arid1a gene and enhancer showing ChIP-seq and CLIP-seq data for bio-YY1 cells, as well as GRO-seq reads for mESCs. FIG. 1D shows a mean read density of YY1 ChIP-seq and CLIP-seq reads at enhancers and promoters of all RefSeq genes in ESCs;

FIG. 2A, FIG. 2B, and FIG. 2C show that YY1 binds to DNA and RNA at promoter-proximal and distal elements in murine embryonic stem cells (ESCs). FIG. 2A shows gene tracks for the Arid1a gene showing ChIP-seq reads for OCT4, SOX2, and NANOG (OSN) and bio-YY1, together with CLIP-seq data for bio-YY1 cells and control ESCs expressing only biotin ligase BirA. Bio-YY1 and control ESCs were previously described (25). Also shown are GRO-seq reads at the Arid1a gene and its enhancer. CLIP-seq and GRO-seq reads that map to Watson and Crick strands of DNA are shown separately. FIG. 2B shows a mean read density of OCT4 ChIP-seq, YY1 ChIP-seq, and YY1 CLIP-seq reads as well as alignment of Oct4 and YY1 motif occurrences at enhancers and promoters of all RefSeq genes in ESCs. The x-axis indicates distance from either the enhancer center (C) or the transcription start site (TSS) in kilobases. The y-axis indicates mean density of uniquely mapped reads or average count of motifs per genomic bin. FIG. 2C shows a heatmap comparing YY1 binding to DNA and RNA at enhancers and promoters of all RefSeq genes in ESCs using ChIP-seq and CLIP-seq data. Enhancers and promoters were ranked based on number of YY1 ChIP-seq reads;

FIG. 3 shows that OCT4 and YY1 are associated with promoters of active genes. Alignment of GRO-seq, OCT4 ChIP-seq, YY1 ChIP-seq, and YY1 CLIP-seq at promoters of transcribed RefSeq genes and at promoters of genes that are not transcribed. The x-axis indicates distance from the TSS in kilobases. The y-axis indicates average density of uniquely mapped reads per genomic bin. Transcribed promoters have more than one GRO-seq read assigned per kilobase of target per million mapped reads (RPKM), whereas not transcribed ones have no GRO-seq and no histone 3 trimethylated at lysine 4 (H3K4me3) ChIP-seq reads;

FIG. 4A, FIG. 4B, FIG. 4C, and FIG. 4D show YY1 CLIP-seq sample preparation and analysis. FIG. 4A shows a schematic of a CLIP-seq protocol used for identification of RNA species bound by YY1 in vivo. FIG. 4B shows a flowchart of a pipeline for identification of RNA regions associated with biotinylated-YY1 (bio-YY1). Each step of the analysis pipeline is indicated in bold font above rectangular boxes. Number of reads or regions at each step of the analysis pipeline is shown in red. FIG. 4C shows a western blot analysis of bio-YY1 purified during the CLIP procedure is shown alongside the autoradiograph of the nitrocellulose membrane obtained after transfer of RNA species UV-crosslinked to the affinity purified bio-YY1 protein from SDS-PAGE to the membrane. Control (Ctrl) CLIP procedure was conducted using cells containing only BirA biotin ligase and no bio-YY1. Rectangular boxes indicate regions of the membrane, which were excised to isolate the RNA. FIG. 4D shows a distribution of lengths of YY1 CLIP-seq reads either containing or lacking deletions demonstrates that deletion reads are longer than reads without deletions consistent with both types of reads being derived from UV-crosslinked RNA species;

FIG. 5 shows that YY1 ChIP-seq and YY1 CLIP-seq reads are distributed similarly among various genomic regions. Stacked bar-plots comparing distributions of YY1 ChIP-seq and YY1 CLIP-seq reads among promoters, enhancers, exons, introns, and all other genomic locations. Distributions of Oct4 ChIP-seq reads, ribo-depleted RNA-seq reads, polyA-selected RNA-seq reads, and GRO-seq reads are shown as controls. Data are presented as either raw reads or reads normalized to the size of these regions in genomic DNA;

FIG. 6A and FIG. 6B show that OCT4 does not bind to RNA in vivo or in vitro. FIG. 6A shows a comparison of YY1 CLIP-seq and OCT4 CLIP-seq reads at enhancers and promoters of all RefSeq genes after background CLIP read densities obtained by sequencing of RNA isolated from control ESCs expressing only biotin ligase BirA were subtracted from CLIP read densities obtained by sequencing of RNA isolated from ESCs expressing biotinylated versions of YY1 (bio-YY1) or OCT4 (bio-OCT4). The x-axis indicates distance from the TSS in kilobases. The y-axis indicates average density of uniquely mapped reads per genomic bin. FIG. 6B shows an EMSA analysis of DNA in complex with recombinant human OCT4 protein (Rec OCT4) in the presence and absence of competitor DNA. A radioactively labeled 40-bp DNA probe containing OCT4 consensus binding motif derived from the enhancer of Lefty1 gene was incubated with 250 nM of the recombinant human OCT4 (Abcam, # ab134876). In competition EMSA, 100-fold molar excess of cold competitor DNA containing the OCT4 motif (Lefty1 DNA comp), cold DNA competitor, in which bases adjacent the OCT4 motif were randomly substituted (mut Lefty1 DNA comp), cold DNA competitor lacking the OCT4 motif (Arid1a DNA comp), or cold RNA competitor (Arid1a RNA comp) was pre-incubated with the recombinant OCT4 before the radioactively labeled DNA probe was added to the binding reaction. The arrows indicate free probe and probe bound by OCT4. Incubation of a 30-nt RNA probe derived from the promoter region of Arid1a gene with 250 nM of the recombinant human OCT4 did not retard the probe;

FIG. 7A and FIG. 7B show that YY1 binds to DNA and RNA in vitro. FIG. 7A shows a left panel: EMSA of YY1-DNA complexes at different concentrations of recombinant YY1. 5 nM of radioactively labeled 30-bp DNA probe derived from the promoter region of Arid1a gene containing a consensus YY1 binding motif (CTCTTCTCTCTTAAAATGGCTGCCTGTCTG; SEQ ID NO: 4) was incubated with increasing concentrations of recombinant murine YY1 protein. Right panel: EMSA of YY1-RNA complexes at different concentrations of recombinant YY1. 5 nM of radioactively labeled 30-nt RNA probe derived from the same region of the Arid1a gene was incubated with increasing concentrations of recombinant YY1 protein. FIG. 7B shows a graph depicting relationship between the fraction of radioactively labeled DNA or RNA probe bound and the concentration of recombinant YY1 in the binding reaction;

FIG. 8A and FIG. 8B show that YY1 binds to DNA probes containing consensus YY1 binding motif in vitro in murine ESC nuclear extracts. FIG. 8A shows an EMSA of YY1-DNA complexes. 10 μl of ESC nuclear extract (NE) was incubated with 30-bp radioactively labeled DNA probes. For supershift assay, 0.5 μl of YY1 antibodies (# sc-7341) was added to the DNA probe pre-incubated with NE. Identity of the DNA probes is shown above the image. The arrows indicate free probe, probe bound by YY1, and supershifted probe. FIG. 8B shows an EMSA analysis of YY1-DNA complexes in the presence and absence of competitor DNA. Radioactively labeled 30-bp DNA probe containing YY1 consensus binding motif derived from the promoter region of Rpl30 gene was incubated with 10 μl of ESC nuclear extract (NE). In competition EMSA, 100-fold molar excess of cold competitor DNA containing YY1 motif (Spec comp) or lacking YY1 motif (Nspec comp) was pre-incubated with NE before the radioactively labeled DNA probe was added to the binding reaction. The arrows indicate free probe and probe bound by YY1;

FIG. 9A, FIG. 9B, and FIG. 9C show that the DNA-binding properties of recombinant murine YY1 are similar to properties of endogenous YY1 present in murine ESC nuclear extracts. FIG. 9A shows a Coomassie Blue staining and Western blot analysis of recombinant murine YY1 used for in vitro studies. FIG. 9B shows an EMSA analysis of DNA in complex with recombinant murine YY1 protein (Rec YY1) in presence and absence of competitor DNA. A radioactively labeled 30-bp DNA probe containing YY1 consensus binding motif derived from the promoter region of Arid1a gene was incubated with 80 nM of the recombinant murine YY1. For supershift assay, 0.5 μl of YY1 antibodies (# sc-7341) was added to the DNA probe pre-incubated with the recombinant protein. In competition EMSA, 100-fold molar excess of cold competitor DNA containing YY1 motif (Spec comp) or lacking YY1 motif (Nspec comp) was pre-incubated with the recombinant YY1 before the radioactively labeled DNA probe was added to the binding reaction. The arrows indicate free probe, probe bound by YY1, probe bound by YY1 fragment, and supershifted probe. FIG. 9C shows an EMSA analysis of YY1-DNA complexes in the presence and absence of competitor RNA. Radioactively labeled 30-bp DNA probe containing YY1 consensus binding motif derived from the promoter region of Rpl30 gene was incubated with 240 nM of the recombinant murine YY1. In competition EMSA, 100-fold molar excess of cold competitor RNA derived from the promoter region of Arid1a gene was pre-incubated with the recombinant murine YY1 before the radioactively labeled DNA probe was added to the binding reaction. The arrows indicate free probe and probe bound by YY1;

FIG. 10A, FIG. 10B, and FIG. 10C show that YY1 binds to some RNA species in vitro. FIG. 10A shows an EMSA analysis of RNA in complex with recombinant murine YY1 protein (Rec YY1). A radioactively labeled 30-nt RNA probe containing sequence derived from the promoter region of Arid1a gene shown in blue above the image or the complementary sequence shown in pink was incubated with 400 nM of the recombinant murine YY1. The arrows indicate free probe and probe bound by YY1. FIG. 10B shows an EMSA analysis of YY1-RNA complexes in presence and absence of competitor RNA. Radioactively labeled 30-nt RNA probe derived from the promoter region of Arid1a gene was incubated with 400 nM of recombinant murine YY1. Sequence of the probe is shown above the image in blue. In competition EMSA, 100-fold molar excess of cold competitor RNA with the same sequence as the radiolabeled RNA (Arid1a RNA A), complementary sequence (Arid1a RNA 1), or a different sequence (Arid1a RNA B) was pre-incubated with the recombinant YY1 before the probe was added to the binding reaction. The arrows indicate free probe and probe bound by YY1. Asterisk indicates the position of double-stranded RNA formed when single-stranded cold RNA was incubated with the complementary labelled RNA probe. FIG. 10C shows an EMSA analysis of YY1-RNA complexes in presence and absence of competitor RNA. Radioactively labeled 30-nt RNA probe derived from the promoter region of Arid1a gene was incubated with 400 nM of recombinant murine YY1. Sequence of the probe and competitor RNA are shown above the image. For supershift assay, 0.5 μl of YY1 antibodies (# sc-7341) was added to the RNA probe. In competition EMSA, 100-fold molar excess of cold competitor RNA with the same sequence as the radiolabeled RNA probe (Arid1a RNA A) or a different sequence (Arid1a RNA B) was pre-incubated with the recombinant YY1 before the probe was added to the binding reaction. Other cold competitors were double-stranded DNA derived from the promoter region of Arid1a gene containing the YY1 motif and the same sequence as the radiolabeled RNA probe (competitor 2) and double-stranded DNA derived from the promoter region of Rpl30 gene containing the YY1 motif (competitor 3). The arrows indicate free probe, probe bound by YY1, and supershifted probe. Asterisk indicates the position of DNA-RNA hybrid probe formed when remnants of unannealed single-stranded DNA present in the cold double-stranded Arid1a DNA competitor interacted with the complementary labelled RNA probe;

FIG. 11A, FIG. 11B, and FIG. 11C show that different regions in YY1 are responsible for binding to DNA and RNA. FIG. 11A shows a cartoon depicting regions of YY1 used in EMSA. Sizes of N-terminal and C-terminal regions of YY1 are drawn to scale. FIG. 11B shows an EMSA of YY1-DNA complexes at different concentrations of recombinant YY1. 5 nM of radioactively labeled 30-bp DNA probe derived from the promoter region of Arid1a gene containing a consensus YY1 binding motif was incubated with increasing concentrations of full-length recombinant murine YY1 protein (FL), the N-terminal portion of YY1 (N-term) lacking the zinc-fingers or the C-terminal portion (C-term) containing the zinc fingers. Concentration of the YY1 protein in binding reactions is displayed above the image. The arrows indicate free probe and probe bound by YY1. FIG. 11C shows an EMSA of YY1-RNA complexes at different concentrations of recombinant YY1. 5 nM of radioactively labeled 30-nt RNA probe derived from the promoter region of Arid1a gene was incubated with increasing concentrations of full-length recombinant murine YY1 protein (FL), the N-terminal portion of YY1 (N-term) lacking the zinc-fingers or the C-terminal portion (C-term) containing the zinc fingers. Concentrations of the YY1 protein in binding reactions are displayed above the image. The arrows indicate free probe and probe bound by YY1;

FIG. 12A, FIG. 12B, FIG. 12C, and FIG. 12D show that perturbation of RNA levels affects YY1 binding to DNA. FIG. 12A shows a cartoon depicting hypothesis that RNA transcribed from regulatory elements enhances occupancy of these elements by TFs capable of binding both DNA and RNA. FIG. 12B (top) shows GRO-seq reads (Wang et al., 2015) at promoters, enhancers, and super-enhancer constituents in cells before (DRB) and after release (Rel) from transcriptional inhibition by DRB. FIG. 12B (bottom) shows YY1 ChIP-seq reads at promoters, enhancers and super-enhancer constituents in cells before (DRB) and after release (Rel) from transcriptional inhibition by DRB. Increase in YY1 binding after release from DRB inhibition is significant: p-value <3.6×10⁻²⁰⁷ for promoters, p-value <1.6×10⁻²¹⁴ for enhancers, p-value <9.8×10⁻³⁷ for super-enhancers. FIG. 12C (top) shows box plots depicting RNA-seq data for ribo-depleted total RNA at promoters, enhancers, and super-enhancers in ESCs after targeting with control (Ctrl) or Exosc3 (ExoKD) shRNA. FIG. 12C (bottom) shows an alignment of YY1 ChIP-seq reads at promoters, enhancers and super-enhancers in ESCs after targeting with control (Ctrl) or Exosc3 (ExoKD) shRNA. The decrease in YY1 binding in ExoKD ESCs is significant: p-value <8.1×10⁻⁹ for promoters, p-value <1.8×10⁻²⁷ for enhancers, p-value <3.3×10⁻⁵ for super-enhancers. FIG. 12D shows a western blot analysis of YY1, OCT4, and histone H3 levels in whole-cell extracts (WCE), nuclei (N), and a nuclear chromatin preparation before and after RNase A treatment. Histone H3 serves as a loading control and OCT4 serves as a negative control. Quantitation of the relative levels of YY1 and OCT4 are noted;

FIG. 13A, FIG. 13B, FIG. 13C, and FIG. 13D show that perturbation of RNA levels using transcription inhibitors affects YY1 binding to DNA. FIG. 13A shows results of YY1 ChIP followed by quantitative PCR (ChIP-qPCR) analysis at the Arid1a enhancer, promoter, and a negative control region in mESCs treated with D-rybofuranosylbenzimidazole (DRB) for various time intervals, and in ESCs at several time points after DRB was removed from the culture media (release after DRB treatment). The y-axis indicates YY1 binding at the specified genomic regions as a fraction of input. FIG. 13B (top) shows an alignment of GRO-seq reads at promoters of all RefSeq genes and at enhancers and super-enhancer constituents in control untreated ESCs and cells treated with DRB (24). (Bottom) Alignment of YY1 ChIP-seq reads at promoters of control untreated ESCs and in cells treated with DRB for 30 min. The x-axis indicates distance from either the TSS or from the enhancer or super-enhancer constituent center FIG. 13C in kilobases. The y-axis indicates average density of uniquely mapped GRO-seq or ChIP-seq reads per genomic bin. Decrease in YY1 binding in presence of DRB is significant: p-value <3.8×10⁻¹⁵ for promoters, p-value <2.4×10-22 for enhancers, p-value <2.7×10⁻³ for super-enhancer constituents. p-values were calculated using the one-tailed t-test. FIG. 13C shows an alignment of GRO-seq and YY1 ChIP-seq reads at promoters of all RefSeq genes as well as at enhancers and super-enhancer constituents in control DMSO-treated ESCs and in cells treated with actinomycin D (ActD) for 6 hrs. The decrease in YY1 binding in the presence of ActD is significant: p-value <4.5×10⁻⁷ for promoters, p-value <1.0×10⁻¹⁰²³ for enhancers, p-value <3.8×10⁻⁸² for super-enhancer constituents. FIG. 13D shows an alignment of YY1 ChIP-seq reads at promoters of all RefSeq genes as well as at enhancers and super-enhancer constituents in control DMSO-treated ESCs and in cells treated with THZ1 or triptolide (TP1) for 6 hrs. Decrease in YY1 binding in presence of THZ1 is significant: p-value <2.1×10⁻⁶ for promoters, p-value <2.5×10⁻²⁷ for enhancers, p-value <1.5×10⁻⁸ for super-enhancer constituents. Decrease in YY1 binding in presence of TP1 is significant: p-value <4.4×10⁻¹⁸ for enhancers, p-value <2.8×10⁻¹ for super-enhancer constituents;

FIG. 14 shows that treatment of ESCs with DRB does not change steady-state levels of YY1. Western blot analysis of protein levels of RNA polymerase II phosphorylated at serine 2 (RNA pol II CTD phospho Ser2), YY1, and OCT4 in control ESCs, in cells treated with DRB within intervals of time shown above the graph, and in ESCs at several time points once DRB was removed from the culture media (release of inhibition). Histone H3 and OCT4 serve as controls;

FIG. 15A and FIG. 15B show RNA-seq and Western blot analyses of ESCs, in which an exosome component was targeted with shRNA. FIG. 15A shows a box plot of changes in expression of all RefSeq transcripts in ESCs targeted with control shRNA against luciferase gene (Ctrl) or with shRNA against Exosc3. RPKM values for RefSeq transcripts and ERCC spike-in probes were calculated using RPKM_count.py (RSeQC). These values were then floored at 0.01 and a pseudocount of 0.1 was added to all entries. Using normalize.loess from the affy R package, RPKM values for the RefSeq transcripts were subsequently normalized using all ERCC probes as the normalization subset, and the distributions were further log-normalized using log.it. RefSeq transcripts were significantly up-regulated in cells targeted with shRNA against Exosc3 relative to control cells (p-value <5.07×10-25). The p-value was calculated using a one-tailed Wilcoxon rank sum test. FIG. 15B shows a western blot analyses of protein levels of exosome component EXOSC3 as well as levels of YY1 and OCT4 in ESCs targeted with control shRNA against luciferase gene (Ctrl) or with shRNA against Exosc3. Levels of GAPDH and ß-Tubulin serve as loading controls. Normalized values are shown below corresponding blots;

FIG. 16A and FIG. 16B show that tethering of RNA adjacent to an YY1 DNA binding site enhances binding of YY1 to the genome in vivo. FIG. 16A shows a strategy for tethering of RNA in the vicinity of an YY1 binding site at enhancers in vivo. FIG. 16B shows a ChIP-qPCR analysis of YY1 binding at six targeted (red) and three not targeted (blue) enhancers in three independent experiments. The y-axis indicates fold change in YY1 binding in ESCs expressing the sgRNA-Arid1a RNA fusion construct relative to cells expressing the control sgRNA targeted to the same locus. The difference in YY1 binding was significant for the targeted enhancers: Klf5 (p-value=0.03), Suz12 (p-value=0.01), E2f3 (p-value=0.01), Nufip2 (p-value=0.03), Cnot6 (p-value=0.03), and Pias1 (p-value=0.01), but not for the not targeted enhancers;

FIG. 17A and FIG. 17B show that RNA sequences compatible with YY1 binding in vitro enhance YY1 binding in vivo when tethered near an YY1 binding site in DNA. FIG. 17A shows a cartoon depicting strategy for tethering of RNA in the vicinity of YY1 binding site at three different enhancers in vivo. Control ESCs were engineered to express catalytically inactive endonuclease Cas9 (dCas9) and guide RNAs (sgRNA) fused to tracrRNA, permitting targeting of dCas9 near an YY1 binding site at the three enhancers. Experimental ESC lines were engineered to express dCas9 with the same sgRNAs and tracrRNA fused to either a 60-nt RNA from the promoter region of Arid1a gene containing RNA sequence compatible with YY1 binding in vitro (RNA A shown in red) or RNA sequence not compatible with YY1 binding in vitro (RNA B shown in blue), permitting the Arid1a RNAs to be targeted near the three enhancers (tethered RNA). FIG. 17B shows a ChIP-qPCR analysis of YY1 binding at three enhancers targeted with a 60-nt RNA from the promoter region of Arid1a gene containing RNA sequence compatible with YY1 binding in vitro (shown in red) and RNA sequence not compatible with YY1 binding in vitro (shown in blue) in control ESCs and in ESCs containing tethered RNAs in three independent experiments. The y-axis indicates fold change in YY1 binding in ESCs expressing the sgRNA Arid1a RNA fusion construct relative to cells expressing the sgRNA targeted to the same locus. The difference in YY1 binding was significant for the enhancers targeted with RNA A: Suz12 enhancer (p-value=0.01), Cnot6 enhancer (p-value=0.03), and E2f3 enhancer (p-value=0.01), but not with RNA B: Suz12 enhancer (p-value=0.4), Cnot6 enhancer (p-value=0.8), and E2f3 enhancer (p-value=0.1);

FIG. 18A, FIG. 18B, and FIG. 18C show analysis of YY1 binding to probes utilized in the competition EMSA. FIG. 18A shows a schematic of probes used in competition EMSA of DNA in complex with recombinant murine YY1. Radioactively labeled 30-bp DNA probe containing YY1 consensus binding motif derived from the promoter region of Rpl30 gene (probe1), DNA probe with the same sequence containing 30-nt RNA derived from the promoter region of Arid1a gene at each 3′end of the DNA (probe 2), or 1:2 mixture of radioactively labeled 30-bp DNA containing YY1 consensus binding motif derived from the promoter region of Rpl30 gene and 30-nt cold RNA derived from the promoter region of Arid1a gene (probe 3) was incubated with various concentrations of the recombinant YY1. FIG. 18B shows an EMSA of YY1-DNA complexes at different concentrations of recombinant YY1. 0.1 pmole of radioactively labeled probes described in FIG. 18A was incubated with increasing concentrations of recombinant murine YY1. Concentrations of the YY1 protein in binding reactions are displayed above the image. Arrows indicate positions of the corresponding free and bound probes. FIG. 18C shows a graph depicting relationship between fraction of the radioactively labeled probes described in FIG. 18A bound by YY1 and concentration of YY1 in binding reactions;

FIG. 19A and FIG. 19B show that DNA containing tethered RNA outcompetes DNA with untethered RNA in competition EMSA. FIG. 19A shows a schematic of probes and competitors used in competition EMSA of DNA in complex with recombinant murine YY1. A radioactively labeled 30-bp DNA probe containing YY1 consensus binding motif derived from the promoter region of Rpl30 gene was incubated with 200 nM of recombinant YY1 in the presence and absence of cold DNA competitor with the same sequence containing 30-nt RNA derived from the promoter region of Arid1a gene at each 3′end of the duplex DNA (competitor 1) or a 1:2 mixture of cold 30-bp DNA containing YY1 consensus binding motif derived from the promoter region of Rpl30 gene and 30-nt RNA derived from the promoter region of Arid1a gene (competitor 2). FIG. 19B (top) shows a EMSA analysis of DNA in complex with recombinant murine YY1 (Rec YY1) in the presence and absence of competitor DNA. Competition assays were conducted with increasing amounts of the two competitors described in FIG. 19A. FIG. 19B (bottom) shows a graph depicting the relationship between fraction of the radioactively labeled DNA probe bound by YY1 and the level of cold competitor in the binding reaction. IC₅₀ values for the two competitors are shown above the graph. The difference between log(IC₅₀) values for the two competitors is significant: p-value <0.05. p-value is estimated using the two-tailed t-test; and

FIG. 20 shows EMSA of various transcription factors in complexes with RNA. 10 μl of ESC nuclear extract (NE) was incubated with 30-nt radioactively labeled RNA probe derived from the promoter region of Arid1a gene, and specific antibodies recognizing various TFs were added to the EMSA reaction to identify TFs bound to the probe. Ronin and CTCF antibodies were able to retard the RNA-protein complexes formed in the NE, suggesting that both Ronin and CTCF were bound to the RNA. For supershift assay, 0.5 μl of KLF4 antibodies (R&D, # AF3158), PRDM14 antibodies (Millipore, #4350), Ronin antibodies (Millipore, # ABE567), REST antibodies (Abcam, #26635), or CTCF antibodies (Millipore, #07-729) was added to the RNA probe pre-incubated with NE. Sequence of the RNA probe is shown above the image. The arrows indicate free probe, bound probe, and supershifted probe.

The patent or application file contains at least one drawing executed in color. Copies of this patent or patent application publication with color drawings will be provided by the Office upon request and payment of the necessary fee.

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

The presently disclosed subject matter provides methods, compositions, and kits for modulating expression of a target gene, and related methods of treating diseases, conditions, and disorders in which aberrant transcription (e.g., increased or decreased) of a target gene is implicated. The presently disclosed subject matter relies on work described herein that demonstrates that RNA transcribed from regulatory elements of a target gene binds to and stabilizes transcription factors occupying those regulatory elements. Without wishing to be bound by theory, it is believed that binding between the RNA transcribed from the regulatory elements of the target gene creates a positive feedback loop, for example, where the transcription factors stimulate local transcription, and newly transcribed nascent RNA reinforces local transcription factor occupancy thereby further stimulating local transcription. Accordingly, in some aspects, the presently disclosed subject matter provides a method of modulating expression of a target gene comprising modulating binding between an RNA transcribed from at least one regulatory element of a target gene and a transcription factor which binds to both the RNA and the regulatory element. In other words, the methods of the presently disclosed subject matter involve modulating transcription of target genes (and expression products of genes) by targeting the RNA transcribed from regulatory elements of target genes whose expression is regulated by transcription factors which are bound by such RNA while the transcription factor occupies the regulatory elements from which the RNA was transcribed. The methods of modulating gene expression disclosed herein may in some embodiments be used for therapeutic purposes, for example, to decrease expression of a target gene whose aberrant or increased transcription is implicated in a disease, condition, or disorder (e.g., a cancer, genetic disorder, etc.) or to increase expression of a target gene whose aberrant or decreased transcription is implicated in a disease, condition, or disorder (e.g., a cancer, genetic disorder, etc.).

### I. Methods for Modulating Expression of a Target Gene

In some embodiments, modulating expression of a target gene comprises modulating binding between a ribonucleic acid (RNA) transcribed from at least one regulatory element of a target gene and a transcription factor which binds to both the RNA and the at least one regulatory element, wherein modulating binding between the RNA and the transcription factor modulates expression of the target gene.

The term “expression” encompasses the processes by which nucleic acids (e.g., DNA) are transcribed to produce RNA, and RNA transcripts are translated into polypeptides. In some embodiments, modulating expression comprises increasing or decreasing levels of transcription. As used herein, “modulate”, or “modulating” refer to changing the rate at which a particular process occurs, inhibiting a particular process, reversing a particular process, and/or preventing the initiation of a particular process, e.g., transcription from a DNA sequence.

The “regulatory element” of the target gene refers to those sequences of the target gene, such as promoters, enhancers, and upstream activating sequences, which help modulate expression of the target gene. The terms “promoter”, “promoter region” or “promoter sequence” refer generally to transcriptional regulatory regions of a gene, which may be found at the 5′ side of the coding region, or within the coding region, or within introns. Typically, a promoter is a DNA regulatory element that is capable of binding the transcriptional machinery and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site, as well as protein binding domains (consensus sequences) responsible for the binding of the transcriptional machinery. In some embodiments, the promoter comprises an active promoter (i.e., a transcriptionally active promoter). In some embodiments, the promoter comprises a promoter that drives transcription of at least one messenger RNA. As used herein, an “active promoter” refers to a promoter that is being used for transcription and generally will be bound by components of the transcription machinery. To determine if a promoter is transcriptionally active, nascent transcripts transcribed from the promoter can be detected by sequencing. In some embodiments, the promoter is a eukaryotic promoter, e.g., a vertebrate promoter, e.g., a mammalian promoter, e.g., a human promoter. As used herein, “enhancer” refers to a short region of DNA to which proteins (e.g., transcription factors) bind to enhance transcription of a gene. In some embodiments, the enhancer comprises an active enhancer. As used herein, “active enhancer” refers to an enhancer that is being used to increase transcription.

As used herein, the term “super-enhancer” refers to genomic regions that contain tightly spaced clusters of enhancers spanning extraordinarily large domains. These “super-enhancers” are occupied by more transcriptional coactivator than the average or median enhancers, exhibit greater activity than average enhancers, and are sufficient to drive high expression of key, cell type-specific genes required to maintain cell identity or disease state (see, for e.g., U.S. Patent Publication Nos. 20140296218 and 20140287932, which are herein incorporated by reference in their entirety). Generally, super-enhancers are formed by at least two enhancers in the genomic region of DNA and are of greater length than the average single enhancer. In some embodiments, the length of the genomic region that forms the super-enhancer is at least an order of magnitude greater than the average single enhancer. In some embodiments, the genomic region spans between about 4 kilobases and about 40 kilobases in length. It should be appreciated, however, that super-enhancers may comprise genomic regions less than 4 kilobases or greater than 40 kilobases in length, as long as the genomic region contains clusters of enhancers that can be occupied when present within a cell by high levels of a transcriptional coactivator (e.g., Mediator), as well as occupied by other enhancer-associated modifications and proteins, including H3K27ac, a histone modification commonly found at enhancers and used to predict regions of enhancers activity.

As used herein, RNA transcribed from a super-enhancer constituent is referred to as super-enhancer constituent RNA. In some embodiments, at least one regulatory element is selected from the group consisting of an enhancer, a promoter, and combinations thereof. In some embodiments, the enhancer is a component of a super-enhancer.

As used herein, the term “transcription factor” refers to a protein that binds to a regulatory element of a target gene to modulate, e.g., increase or decrease, expression of the target gene. The presently disclosed subject matter contemplates the use of any transcription factor that is capable of simultaneously binding to both DNA sequences of regulatory elements and RNA sequences transcribed from those regulatory elements. As used herein, “simultaneously binding” of a transcription factor to both DNA sequences of regulatory elements and RNA sequences transcribed from those regulatory elements means that the transcription factor is capable of binding both the DNA sequence and the RNA sequence at the same time for at least a portion of a related activity (e.g., transcription of the target gene to produce an mRNA encoding a protein) even though the transcription factor might not be bound to both the DNA sequence and the RNA sequence at the same time throughout the related activity. For the avoidance of doubt, simultaneous binding contemplates situations in which the DNA sequence is occupied by the transcription factor before the transcribed RNA sequence is bound, as well as those in which the transcribed RNA sequence is bound even though the transcription factor is not occupying the DNA sequence.

Non-limiting examples of transcription factors that can bind both DNA, such as a regulatory element, and RNA include, but are not limited to, Yin-Yang 1 (YY1), Krueppel-like factor 4 (KLF4), Ronin (Thap11), RE1-silencing transcription factor (REST), PR domain zinc finger protein 14 (PRDM14), CCCTC-binding factor (CTCF), p53, Signal transducer and activator of transcription 1 (STAT1), TLS/FUS, BRCA1, DLX2, ESR1, FUS, KIN, KU, NACA, NCL, NFKB1, NFYA, NR3C1, RARA, RUNX1, SOX2, TCF7, and TP53. In some embodiments, the transcription factor is selected from the group consisting of Yin-Yang 1 (YY1), Krueppel-like factor 4 (KLF4), Ronin (Thap11), RE1-silencing transcription factor (REST), PR domain zinc finger protein 14 (PRDM14), CCCTC-binding factor (CTCF), Signal transducer and activator of transcription 1 (STAT1), and TLS/FUS, BRCA1, DLX2, ESR1, KIN, KU, NACA, NCL, NFKB1, NFYA, NR3C1, RARA, RUNX1, SOX2, TCF7, and TP53 (p53).

In some embodiments, the transcription factor is Yin-Yang 1 (YY1). Transcription factor YY1 includes a C-terminal DNA binding domain, which binds to one or more DNA consensus motifs, a C2H2-type 1 zinc finger, a C2H2-type 2 zinc finger, a C2H2-type 3 zinc finger, and a C2H2-type 4 zinc finger. In some embodiments, the transcription factor is KLF4. Transcription factor KLF4 includes a C-terminal DNA binding domain, which binds one or more DNA consensus motifs, a C2H2-type 1 zinc finger, a C2H2-type 2 zinc finger, and a C2H2-type 3 zinc finger. In some embodiments, the transcription factor is REST. Transcription factor REST includes a DNA binding domain, which binds one or more DNA consensus motifs, and a beta beta alpha-zinc finger. In some embodiments, the transcription factor is Ronin. In some embodiments, the transcription factor is PRDM14. In some embodiments, the transcription factor is CTCF. Transcription factor CTCF includes a DNA binding domain, which binds one or more DNA consensus motifs, a C2H2-type 1 zinc finger, a C2H2-type 2 zinc finger, a C2H2-type 3 zinc finger, a C2H2-type 4 zinc finger, a C2H2-type 5 zinc finger, a C2H2-type 6 zinc finger, a C2H2-type 7 zinc finger, a C2H2-type 8 zinc finger, a C2H2-type 9 zinc finger, a C2H2-type 10 zinc finger, and a C2H2-type 11 zinc finger. In some embodiments, the transcription factor is TP53. Transcription factor TP53 includes an N-terminal DNA binding domain, which binds one or more DNA consensus motifs, zinc-coordinating, and a helix-loop-helix DNA binding domain. In some embodiments, the transcription factor is STAT-1. Transcription factor STAT1 includes a DNA binding domain, which binds one or more DNA consensus motifs, and a Ig-fold (STAT). In some embodiments, the transcription factor is FUS. Transcription factor FUS includes a DNA binding domain, which binds one or more DNA consensus motifs, a winged helix-turn-helix, a EcoRII fold, and a TF-B3 DNA binding region. In some embodiments, the transcription factor is BRCA1. Transcription factor BRCA1 includes a C-terminal DNA binding domain, which binds one or more DNA consensus motifs, and a RING type zinc finger region. In some embodiments, the transcription factor is DLX2. Transcription factor DLX2 includes a DNA binding domain, which binds one or more DNA consensus motifs, and a helix-turn-helix (homeobox DNA binding region). In some embodiments, the transcription factor is ESR1. Transcription factor ESR1 includes a DNA binding domain, which binds one or more DNA consensus motifs, a zinc-coordinating, nuclear receptor DNA binding region, and a NR C4 type zinc finger region. In some embodiments, the transcription factor is FUS. Transcription factor FUS includes a DNA binding domain, which binds one or more DNA consensus motifs, a winged helix-turn-helix, EcoRII fold, and a TF-B3 DNA binding region. In some embodiments, the transcription factor is KIN. Transcription factor KIN includes a DNA binding domain, which binds one or more DNA consensus motifs, a leucine zipper, and a STAT. In some embodiments, the transcription factor is KU. Transcription factor KU includes a DNA binding domain, which binds one or more DNA consensus motifs, a helix-turn-helix (homeobox-DNA binding region). In some embodiments, the transcription factor is NACA. In some embodiments, the transcription factor is NCL. Transcription factor NCL includes an N-terminal DNA binding domain, which binds one or more DNA consensus motifs, and a zinc-coordinating (GATA). In some embodiments, the transcription factor is NFKB1. Transcription factor NFKB1 includes an N-terminal DNA binding domain, which binds one or more DNA consensus motifs, and a Rel homology DNA binding domain. In some embodiments, the transcription factor is NFYA. Transcription factor NFYA includes a C-terminal DNA binding domain, which binds one or more DNA consensus motifs, a NFYA/HAP2 type DNA binding region, and a CCAAT-binding site. In some embodiments, the transcription factor is NR3C1. Transcription factor NR3C1 includes a DNA binding domain, which binds one or more DNA consensus motifs, and a zinc-coordinating (hormone-nuclear receptor). In some embodiments, the transcription factor is RARA. Transcription factor RARA includes an N-terminal DNA binding domain, which binds one or more DNA consensus motifs, and a zinc-coordinating (hormone-nuclear receptor). In some embodiments, the transcription factor is RUNX1. Transcription factor RUNX1 includes an N-terminal DNA binding domain, which binds one or more DNA consensus motifs, and a Ig-fold (Runt). In some embodiments, the transcription factor is SOX2. Transcription factor SOX2 includes a C-terminal DNA binding domain, which binds one or more DNA consensus motifs, and a helix-turn-helix (homeo DNA binding region). In some embodiments, the transcription factor is TCF7. Transcription factor TCF7 includes a DNA binding domain, which binds one or more DNA consensus motifs, and an alpha-helix (HMG box). It should be appreciated that the aforementioned transcription factors and DNA-RNA binding proteins listed in Table 1 can be excluded from some embodiments.

The experiments described in Example 1 were performed in murine embryonic stem cells (mESCs). However, the skilled artisan will appreciate the protocols described herein can be modified for use in other organisms, as well as other cell types. Further, it should be appreciated that the protocols described herein can be readily adapted to identify relevant transcription factors, including but not limited to the transcription factors and DRBPs disclosed herein, in other organisms (subjects) and cell types. It is expected that transcription factors which bind to at least one regulatory element and RNA transcribed from the at least one regulatory element may differ in different cell types within the same organisms, and such transcription factors may further differ between differ organisms and different cell types within those organisms. Accordingly, the presently disclosed subject matter contemplates the use of any transcription factor in any cell type in any organism, as long as the transcription factor simultaneously binds to the at least one regulatory element and the RNA transcribed from the at least one regulatory element.

For example, identification of suitable transcription factors in any particular cell or organism can be identified by selecting an organism of interest, selecting a cell type of interest, identifying active regulatory elements (e.g., enhancers, promoters, super-enhancer constituents, etc.) throughout the genome of the cell within that particular organism, identifying transcription factors that interact with those active regulatory elements, identifying transcription factors that bind to those regulatory elements and RNA transcribed from those regulatory elements, and assessing whether modulation of the RNA transcribed from those regulatory elements modulates transcription of one or more target genes regulated by those regulatory elements (i.e., whether binding of the transcribed RNA stabilizes occupancy of the transcription factor at the at least one regulatory element), wherein a transcription factor which binds to the at least one regulatory element and the RNA transcribed from the at least one regulatory element and stabilizes occupancy of the transcription factor at the at least one regulatory element is a suitable candidate transcription factor for further evaluation in accordance with the experimental protocols described herein. It should be appreciated that a variety of publicly available resources are available to assist with genome-wide identification of active promoters and enhancers in various organisms and cell types. For example, a promoter-level mammalian expression atlas generated by the FANTOM Consortium and the RIKEN PMI and CLST (DGT) (Forrest et al., “A promoter-level mammalian expression atlas,” Nature. 2014; 507(7493):462-470, which is incorporated herein by reference in its entirety) describes the FANTOM5 promoter atlas, as well as mammalian promoter architectures, expression levels and tissue specificity, promoter conversion between human and mouse, features of cell-type-specific promoters, key cell-type-specific transcription factors, and inferring function from expression profiles, which can be used to identify active promoters in a specific cell type in mammals to identify transcription factors of use herein. Similarly, an integrated encyclopedia of DNA elements in the human genome generated by the ENCODE Project Consortium (The ENCODE Project Consortium, “An Integrated Encyclopedia of DNA Elements in the Human Genome,” Nature. 2012; 489(7414):57-74, which is incorporated by reference in its entirety) describes a comprehensive catalog of human protein-coding and non-coding RNAs as well as pseudogenes (GENCODE reference gene set), an extensive RNA expression catalog, regions bound by transcription factors, transcriptional machinery, and other proteins, DNaseI hypersensitivity sites, footprints and nucleosome-depleted regions, regions of histone modifications, DNA methylation, chromosome-interacting regions, ENCODE assays which directly or indirectly provide information about the action of promoters, transcription factor-binding sites, sequence variants, for example common variants associated with human diseases and phenotypes, that can be used to identify active promoters and enhancers in specific cell types in humans to identify transcription factors of use herein. As another example, an atlas of active enhancers across human cell types and tissues is available (Andersson, et al., “An atlas of active enhancers across human cell types and tissues,” Nature. 2014; 507:455-461), which can also be used to identify active enhancers in specific cell types in humans to assist with identifying transcription factors of use herein.

Other transcription factors that bind both DNA and RNA can be identified using methods known to a person with ordinary skill in the art, such as cross-linking immunoprecipitation (CLIP) and chromatin immunoprecipation (ChIP).

TABLE 1
DNA-RNA Binding Proteins

| | DNA bound by | RNA bound | Structural | General protein | DNA binding |
|---|---|---|---|---|---|
| Protein/Gene | protein | by protein | domain | function | motifs |
| BRCA1 | Branched DNA | miRNA | Not fully | DNA Repair | RING type |
| | and dsDNA | | characterized; | Apoptosis | zinc finger |
| | | | AAs 230-534 | | region |
| CTCF | dsDNA | mRNA, | 11 C2H2 zinc | Transcriptional | C2H2-type 1 |
| | | IncRFNA | fingers | regulation | zinc finger, |
| | | | | | C2H2-type 2 |
| | | | | | zinc finger, |
| | | | | | C2H2-type 3 |
| | | | | | zinc finger, |
| | | | | | C2H2-type 4 |
| | | | | | zinc finger |
| | | | | | C2H2-type 5 |
| | | | | | zinc finger, |
| | | | | | C2H2-type 6 |
| | | | | | zinc finger, |
| | | | | | C2H2-type 7 |
| | | | | | zinc finger, |
| | | | | | C2H2-type 8 |
| | | | | | zinc finger |
| | | | | | C2H2-type 9 |
| | | | | | zinc finger, |
| | | | | | C2H2-type 10 |
| | | | | | zinc finger, |
| | | | | | C2H2-type 11 |
| | | | | | zinc finger |
| DLX2 | dsDNA (ex: | Evf-2 | Homeodomain | Embryonic | Helix-turn- |
| | Wnt | ncRNA | | development | helix |
| | enhancer) | (ssRNA) | | Lymphocyte | (homeobox |
| | | | | maturation | DNA binding |
| | | | | Transcriptional | region) |
| | | | | regulation | |
| ESR1 (Estrogen | dsDNA | G-rich | Two C4 Zinc | Female | Zinc- |
| receptor) | (estrogen | sequences | fingers | reproduction | coordinating, |
| | response | | | Hormone responses | Nuclear |
| | elements) | | | Transcriptional | receptor DNA |
| | | | | regulation | binding |
| | | | | | region, NR C4 |
| | | | | | type zinc |
| | | | | | finger region |
| FUS (TLS) | ssDNA and | ncRNAs | RRM | Transcriptional | Winged helix- |
| | dsDNA | mRNA | Zinc finger | regulation | turn-helix, |
| | | | (RanBP2-type) | Neurodegeneration | EcoRII fold, |
| | | | | | TF-B3 DNA |
| | | | | | binding |
| | | | | | region |
| KIN (KIN17) | dsDNA | G-rich RNA | RNA: C- | DNA replication | Leucine |
| | | | terminal SH3- | DNA damage | zipper, |
| | | | like domains | response | Other (STAT) |
| | | | DNA: C2H2 Zn | | |
| | | | finger | | |
| KLF4 | dsDNA | | Three zinc fingers | Transcription factor | C2H2-type 1 |
| | | | in very C- | | zinc finger, |
| | | | terminal end | | C2H2-type 2 |
| | | | | | zinc finger, |
| | | | | | C2H2-type 3 |
| KU | dsDNA | TLC1 | Ku70/Ku80 ring | Non-homologous | Helix-turn- |
| (Saccharomyces) | Damaged DNA | telomerase | around DNA | end-joining and | helix, |
| | | RNA | | DNA repair | (homeobox- |
| | | mRNA | | Telomere | DNA binding |
| | | | | maintenance | region) |
| NACA (α-NAC) | dsDNA | rRNA | NAC domain | Transcriptional | Unknown |
| | ssDNA | tRNA | | regulation | |
| | | | | Chaperone of | |
| | | | | nascent | |
| | | | | polypeptides | |
| NCL (Nucleolin) | dsDNA (ex: | mRNA (ex: | Four RRMs and | RNA metabolism | Zinc- |
| | CD34 | APP 3′ UTR) | RGG domain | DNA replication | coordinating |
| | promoter) | | | Ribosome assembly | (GATA) |
| NFKB1 (and RELA) | dsDNA (ex: IFN- | RNA | Rel homology | Immune signaling | Rel homology |
| | β) | aptamers | domain | | DNA binding |
| | | Lethe RNA | | | domain |
| NFYA (NF-YA) | dsDNA (CCAAT | lincRNA | Binds along with | DNA damage and | NFYA/HAP2 |
| | boxes) | PANDA | NF-YB and NF-YC, | apoptosis | type DNA |
| | | | which contain | | binding |
| | | | histone-like | | region |
| | | | domains | | CCAAT- |
| | | | | | binding |
| NR3C1 | dsDNA | mRNA | Two C4 Zinc | Hormone responses | Zinc- |
| (Glucocorticoid | (glucocorticoid | lincRNA | fingers | Transcriptional | coordinating, |
| receptor) | response | Gas5 | | regulation | Zinc- |
| | elements) | tRNA | | Immunity | coordinating |
| | | | | Metabolism | (Hormone- |
| | | | | | nuclear |
| | | | | | receptor) |
| PRDM14 | dsDNA | | Six C2H2 zinc | Transcriptional | C2H2-type 1 |
| | | | fingers at C- | regulation | zinc finger, |
| | | | terminal end | | C2H2-type 2 |
| | | | | | zinc finger, |
| | | | | | C2H2-type 3 |
| | | | | | zinc finger, |
| | | | | | C2H2-type 4 |
| | | | | | zinc finger |
| | | | | | C2H2-type 5 |
| | | | | | zinc finger, |
| | | | | | C2H2-type 6 |
| | | | | | zinc finger |
| RARA (RARα) | dsDNA | 5′ UTRs (ex: | CCCC Zinc finger | Transcriptional | Zinc- |
| | | GluR1) | and F domain | control | coordinating, |
| | | | | Development and | Zinc- |
| | | | | cell differentiation | coordinating |
| | | | | | (Hormone- |
| | | | | | nuclear |
| | | | | | receptor) |
| RE1-silencing | dsDNA | | Nine C2H2 zinc | Transcriptional | C2H2-type 1 |
| transcription | | | fingers | regulation | zinc finger, |
| factor (REST) | | | | Tumor suppressor | C2H2-type 2 |
| | | | | | zinc finger, |
| | | | | | C2H2-type 3 |
| | | | | | zinc finger, |
| | | | | | C2H2-type 4 |
| | | | | | zinc finger |
| | | | | | C2H2-type 5 |
| | | | | | zinc finger, |
| | | | | | C2H2-type 6 |
| | | | | | zinc finger, |
| | | | | | C2H2-type 7 |
| | | | | | zinc finger, |
| | | | | | C2H2-type 8 |
| | | | | | zinc finger |
| | | | | | C2H2-type 9 |
| | | | | | zinc finger |
| Ronin (Thap11) | dsDNA | | THAP-type zinc | Transcriptional | Putative |
| | | | finger at N- | regulation | HCFC1- |
| | | | terminal end | | binding motif |
| RUNX1 (AML1) | dsDNA | RNA | Runt domain | Hematopoiesis | Ig-fold (Runt) |
| | (ex: ALOX2 | aptamers | | Transcriptional | |
| | promoter) | | | regulation | |
| | | | | Cell proliferation | |
| SOX2 | dsDNA | IncRNAs | HMG Box | Pluripotency | Helix-turn- |
| | | (ex: RMST) | | | helix (homeo |
| | | | | | DNA binding |
| | | | | | region), Other |
| | | | | | alpha-helix |
| STAT1 | dsDNA (ex: IFN- | TSU RNA | Immunoglobulin | Transcriptional | Ig-fold (STAT) |
| | γ activation | | fold (p53 and NF- | regulation | |
| | sites (GAS)) | | κB-like) | Immune signaling | |
| TCF7 (TCF-1) | dsDNA | RNA | HMG box | T cell development | Other alpha- |
| | | aptamers | | Transcriptional | helix (HMG |
| | | | | regulation | box) |
| | | | | Pluripotency | |
| TP53 (p53) | ssDNA | ssRNA | Core DNA- | DNA damage | Zinc- |
| | dsDNA | 5′ and 3′ | binding domain; | response | coordinating, |
| | Damaged DNA | UTRS (ex: | β-sandwich | Transcriptional | Helix-loop- |
| | | PAI-1) | containing | activation | helix DNA |
| | | | | Apoptosis | binding |
| | | | | | domain |
| YY1 | dsDNA (ex: grp | lincRNA Xist | Four C2H2 zinc | Transcriptional | C2H2-type 1 |
| | promoter) | | fingers | regulation | zinc finger, |
| | | | | T cell homeostasis | C2H2-type 2 |
| | | | | | zinc finger, |
| | | | | | C2H2-type 3 |
| | | | | | zinc finger, |
| | | | | | C2H2-type 4 |
| | | | | | zinc finger |

In some embodiments, any region of the transcription factor can bind to the RNA or at least one regulatory element as long as the RNA and the regulatory element are not binding in the same region and therefore competing for binding to the transcription factor. As shown in Table 1, DNA binding motifs can occur throughout a transcription factor and are not limited to one specific region. In some embodiments, the transcription factor comprises an N-terminal region and a C-terminal region, wherein the N-terminal region binds to either the RNA or the at least one regulatory element, and the C-terminal region binds to the RNA or the at least one regulatory element which is not bound to the N-terminal region. In some embodiments, a region (e.g., one or more domains) of the transcription factor between the C-terminal region and the N-terminal region (i.e., central region) binds to the RNA and/or at least one regulatory element.

In some embodiments, either the N-terminal region or the C-terminal region comprises a DNA binding domain selected from the group consisting of a zinc finger, leucine zipper, helix-turn-helix, winged helix-turn-helix, helix-loop-helix, HMG-box, and OB-fold. In some embodiments, either the N-terminal region or the C-terminal region comprises an RNA binding domain. Non-limiting examples of RNA binding domains contemplated herein, such as the RNA Recognition Motif (RRM), the K homology (KH) domain, the CCCH zinc finger domain, the Like Sm domain, the Cold-shock domain, the PUA domain, the Ribosomal protein S1-like domain, the Surp module/SWAP domain, the Lupus La RNA-binding domain, the PWI domain, the YTH domain, the THUMP domain, the Pumilio-like domain, the Sterile alpha motif, the C2H2 zinc finger domain, the RNP-1 motif, and the RNP-2 motif can be found in the database of RNA-binding protein specificities (RBPDB; http://rbpdb.ccbr.utoronto.ca). In some embodiments, at least one of the N-terminal region, the central region, or the C-terminal region of the transcription factor comprises a DNA binding domain, and at least one of the N-terminal region, the central region, or the C-terminal region lacking the DNA binding domain contains an RNA binding domain.

In some embodiments, the RNA is a non-coding RNA selected from the group consisting of enhancer RNA, promoter RNA, and super-constituent RNA. In some embodiments, the enhancer RNA is transcribed from an enhancer that is a super-enhancer. As used herein, a “non-coding RNA” is a RNA that is not translated into protein. In some embodiments, the RNA is nascent RNA which may still bound to RNA polymerase, such as RNA polymerase II. In some embodiments, the RNA is RNA that has been fully released from the RNA polymerase (e.g., RNA subject to degradation by the exosome).

In some embodiments, modulating binding comprises promoting binding between the RNA and the transcription factor. As used herein, “binding” between the RNA and the transcription factor includes binding via non-covalent interactions, such as van der Waals interactions, electrostatic interactions (salt bridges), dipolar interactions (hydrogen bonding), and entropic effects (hydrophobic interactions). It is believed that promoting binding between the RNA and the transcription factor stabilizes occupancy of the transcription factor at the at least one regulatory element, thereby increasing expression of the target gene (e.g., increasing transcription).

Accordingly, in some embodiments, the disclosure provides a method of increasing expression of a target gene, the method comprising promoting binding between a ribonucleic acid (RNA) and a transcription factor which binds to both the RNA and the at least one regulatory element, wherein promoting binding between the RNA and the transcription factor stabilizes occupancy of the transcription factor at the at least one regulatory element, thereby increasing expression of the target gene.

The term “stabilizes occupancy” means that the transcribed RNA keeps the transcription factor sufficiently bound to, or close enough to, the at least one regulatory element for the transcription of the target gene to occur, for example, by increasing the binding affinity or apparent binding affinity of the transcription factor to one of its consensus motifs in the at least one regulatory element. Without wishing to be bound by theory, it is believed that the RNA transcribed from the at least one regulatory element captures the transcription factor via relatively weak interactions as it is dissociating from the at least one regulatory element, which allows the transcription factor to rebind to nearby DNA sequences, thus creating a kinetic sink that increases transcription factor occupancy on the at least one regulatory element. In some embodiments, stabilizing occupancy of the transcription factor at the at least one regulatory element increases the level of transcription of the target gene by at least about 1-fold, 1.1-fold, 1.2-fold, 1.3-fold, 1.4-fold 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold or more, e.g., within a cell, tissue, or subject. In some embodiments, stabilizing occupancy of the transcription factor at the at least one regulatory element increases the level of transcription of the target gene by between 1-fold and 5-fold. In some embodiments, stabilizing occupancy of the transcription factor at the at least one regulatory element increases the level of transcription of the target gene by between 1-fold and 2-fold. In some embodiments, the binding affinity or the apparent binding affinity of the transcription factor for at least one regulatory element is increased by about 1-fold, 1.1-fold, 1.2-fold, 1.3-fold, 1.4-fold 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold or more, e.g., within a cell, tissue, or subject. In some embodiments, the binding affinity or the apparent binding affinity of the transcription factor for at least one regulatory element is increased by between 1-fold and 5-fold. In some embodiments, the binding affinity or the apparent binding affinity of the transcription factor for at least one regulatory element is increased by between 1-fold and 2-fold.

In some embodiments, determining whether promoting binding between an RNA and a transcription factor stabilizes occupancy of the transcription factor at the at least one regulatory element and/or increases transcription of the target gene comprising the at least one regulatory element can be achieved by detecting levels of mRNA encoded by the target gene. In some embodiments, determining whether promoting binding between an RNA and a transcription factor stabilizes occupancy of the transcription factor at the at least one regulatory element and/or increases transcription of the target gene comprising the at least one regulatory element can be achieved by detecting levels and/or activity of protein encoded by the target gene.

A variety of methods for detecting levels of mRNA and/or levels and/or activity of protein expressed by a target gene are well known in the art. The presently disclosed subject matter contemplates the use of any such method. Examples of such suitable methods include RNA-Seq, RT-PCR, real-time PCR, Northern blotting, Western blotting, in situ hybridization, oligonucleotide arrays (e.g., microarray) or chips, to name more than a few. In some embodiments determining whether promoting binding between an RNA and a transcription factor stabilizes occupancy of the transcription factor at the at least one regulatory element and/or increases transcription of the target gene comprising the at least one regulatory element may be performed using a reporter construct comprising a nucleic acid sequence encoding a reporter protein operably linked to the regulatory element of interest. One could detect the reporter protein as an indicator of transcription driven by the regulatory element (e.g., in the presence of a test agent being tested for its ability to interfere with or promote binding between the RNA and the transcription factor). It should be appreciated that such reporter construct could also be used to determine whether inhibiting binding between an RNA and a transcription factor destabilizes occupancy of the transcription factor at the at least one regulatory element and/or decreases transcription of the target gene comprising the at least one regulatory element. In some embodiments, a fluorescent reporter RNA can be used as an indicator of transcription driven by the regulatory element (e.g., in the presence of a test agent being tested for its ability to interfere with or promote binding between the RNA and the transcription factor). Examples of suitable fluorescent reporter RNAs include RNA mimics of green fluorescent protein (see, e.g., Paige et al., “RNA Mimics of Green Fluorescent Protein,” Science. 2011 (333): 642-646, which is incorporated herein by reference). It should be appreciated that transcription of the target gene can be modulated by promoting binding between the RNA transcribed from the at least one regulatory element, as well as by promoting binding between RNA that is not transcribed from the at least one regulatory element but nevertheless is capable of binding to the transcription factor either at the same RNA binding domain at which the transcription factor binds the RNA transcribed from the at least one regulatory element, or at another site of the transcription factor that is distinct from the DNA binding domain (and/or does not interfere with binding between the transcription factor and the at least one regulatory element). That is, the presently disclosed subject matter contemplates the use of any RNA that is capable of binding to the transcription factor in a way that stabilizes occupancy of the transcription factor at the at least one regulatory element.

In some embodiments, promoting binding between the RNA and the transcription factor comprises tethering an RNA that binds to the transcription factor to a DNA sequence proximal to the at least one regulatory element. In some embodiments, the RNA is tethered to a DNA sequence proximal to at least one regulatory element. In some embodiments, the RNA is tethered within at least one regulatory element. In these embodiments, the RNA that is tethered is not the RNA transcribed from a regulatory element or an RNA that is released by RNA polymerase. Rather, the RNA that is tethered is a synthetic RNA that binds to the transcription factor in a way that stabilizes the transcription factor. In some embodiments, the tethered RNA is homologous to the RNA transcribed from a regulatory element.

The term “homologous” means that a polynucleotide, such as an RNA, comprises a sequence that has a desired identity, for example, at least 60% identity, preferably at least 70% sequence identity, more preferably at least 80%, still more preferably at least 90% and even more preferably at least 95%, compared to a reference sequence. In some embodiments, the synthetic RNA is at least 81% identical to RNA transcribed from the at least one regulatory element. In some embodiments, the synthetic RNA is at least 82% identical to RNA transcribed from the at least one regulatory element. In some embodiments, the synthetic RNA is at least 83% identical to RNA transcribed from the at least one regulatory element. In some embodiments, the synthetic RNA is at least 84% identical to RNA transcribed from the at least one regulatory element. In some embodiments, the synthetic RNA is at least 85% identical to RNA transcribed from the at least one regulatory element. In some embodiments, the synthetic RNA is at least 86% identical to RNA transcribed from the at least one regulatory element. In some embodiments, the synthetic RNA is at least 87% identical to RNA transcribed from the at least one regulatory element. In some embodiments, the synthetic RNA is at least 88% identical to RNA transcribed from the at least one regulatory element. In some embodiments, the synthetic RNA is at least 89% identical to RNA transcribed from the at least one regulatory element. In some embodiments, the synthetic RNA is at least 90% identical to RNA transcribed from the at least one regulatory element. In some embodiments, the synthetic RNA is at least 91% identical to RNA transcribed from the at least one regulatory element. In some embodiments, the synthetic RNA is at least 92% identical to RNA transcribed from the at least one regulatory element. In some embodiments, the synthetic RNA is at least 93% identical to RNA transcribed from the at least one regulatory element. In some embodiments, the synthetic RNA is at least 94% identical to RNA transcribed from the at least one regulatory element. In some embodiments, the synthetic RNA is at least 95% identical to RNA transcribed from the at least one regulatory element. In some embodiments, the synthetic RNA is at least 96% identical to RNA transcribed from the at least one regulatory element. In some embodiments, the synthetic RNA is at least 96% identical to RNA transcribed from the at least one regulatory element. In some embodiments, the synthetic RNA is at least 97% identical to RNA transcribed from the at least one regulatory element. In some embodiments, the synthetic RNA is at least 98% identical to RNA transcribed from the at least one regulatory element. In some embodiments, the synthetic RNA is at least 99% identical to RNA transcribed from the at least one regulatory element. In some embodiments, the synthetic RNA comprises at least one, two, three, four, five, six, seven, eight, nine, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 175, 200, 225, 250, or more, mismatched nucleotides as compared to the RNA transcribed from the at least one regulatory element. Determining optimal alignment is within the purview of one of skill in the art. For example, there are publically and commercially available alignment algorithms and programs such as, but not limited to, ClustalW, Smith-Waterman in matlab, Bowtie, Geneious, Biopython and SeqMan.

The term ‘tethered“, as in tethered RNA, refers to fastening or connecting of the RNA, such as to a DNA sequence. For example, fastening of the RNA can be achieved using a catalytically inactive Cas9 protein of the CRISPR/Cas system which utilizes a fusion RNA construct comprising a guide RNA and the RNA to target the RNA to a DNA sequence in proximity to the at least one regulatory element where the RNA can bind the transcription factor occupying the at least one regulatory element and stabilize occupancy of the transcription factor. For example, in some embodiments, an RNA molecule is within a distance of a regulatory element and/or the transcription factor such that the RNA is capable of interacting with or binding to the transcription factor. As another example, tethering may involve covalently binding an RNA to the end of another nucleic acid molecule, such as a DNA molecule.

In some embodiments, to assay whether tethered RNA increases binding affinity of the transcription factor to a DNA sequence, such as a DNA sequence comprising its DNA binding motif, labeled DNA probe comprising the DNA binding motif can be incubated with the transcription factor in the presence of increasing concentrations of unlabeled competitor DNA with tethered or untethered RNA, the transcription factor-DNA complexes can be separated from the unlabeled nucleic acid, and the amount of labeled DNA that remains bound can be quantified. If the DNA containing the tethered RNA outcompetes the DNA without the tethered RNA for transcription factor binding, it is indicative that the tethered RNA increases binding affinity of the transcription factor for its DNA binding motif.

In some embodiments, modulating binding comprises interfering with binding between the RNA and the transcription factor. In some embodiments, the disclosure provides a method of decreasing expression of a target gene, the method comprising interfering with binding between a ribonucleic acid (RNA) transcribed from at least one regulatory element of a target gene and a transcription factor which binds to both the RNA and the at least one regulatory element, wherein interfering with binding between the RNA and the transcription factor destabilizes occupancy of the transcription factor at the at least one regulatory element, thereby decreasing expression of the target gene.

The term “destabilizes occupancy” means that the transcribed RNA weakens the attraction or interaction between the transcription factor and the at least one regulatory element (e.g., by decreasing the binding affinity or apparent binding affinity of the transcription factor and the at least one regulatory element) and/or reduces the local concentration of the transcription factor in proximity to the at least one regulatory element, such that the transcription factor does not remain sufficiently bound to, or present at a sufficient concentration in proximity to, the at least one regulatory element for transcription of the target gene to occur. In some embodiments, destabilizing occupancy of the transcription factor at the at least one regulatory element decreases the level of transcription of the target gene by at least about 5%, 10%, 15%, 20%, 25%, 30%, 33%, 35%, 40%, 45%, 50%, 55%, 60%, 66%, 70%, 75%, 80%, 85%, 90%, or 95% or more, e.g., within a cell, tissue, or subject. In some embodiments, the level of transcription of the target gene is decreased within the cell by 100% (i.e., complete inhibition of transcription of the target gene). In some embodiments, the binding affinity or the apparent binding affinity of the transcription factor for at least one regulatory element is reduced by at least about 5%, 10%, 15%, 20%, 25%, 30%, 33%, 35%, 40%, 45%, 50%, 55%, 60%, 66%, 70%, 75%, 80%, 85%, 90%, or 95% or more, e.g., within a cell, tissue, or subject.

In some embodiments, determining whether interfering with binding between an RNA and a transcription factor destabilizes occupancy of the transcription factor at the at least one regulatory element and/or decreases transcription of the target gene comprising the at least one regulatory element can be achieved by detecting levels of mRNA encoded by the target gene. In some embodiments, determining whether interfering with binding between an RNA and a transcription factor destabilizes occupancy of the transcription factor at the at least one regulatory element and/or decreases transcription of the target gene comprising the at least one regulatory element can be achieved by detecting levels and/or activity of protein encoded by the target gene.

In some embodiments, modulating expression of the target gene occurs in vitro or ex vivo. In some embodiments, modulating expression of the target gene comprises contacting a cell with an effective amount of a composition and/or agent which promotes binding between the RNA and the transcription factor. In some embodiments, modulating expression of the target gene comprises contacting a cell with an effective amount of a composition and/or agent which interferes with binding between the RNA and the transcription factor. As used herein “contacting the cell” and the like, refers to any means of introducing an agent into a target cell in vitro or in vivo, including by chemical and physical means, whether directly or indirectly or whether the agent physically contacts the cell directly or is introduced into an environment (e.g., culture medium) in which the cell is present or to which the cell is added. Contacting also is intended to encompass methods of exposing a cell, delivering to a cell, or ‘loading’ a cell with an agent by viral or non-viral vectors, and wherein such agent is bioactive upon delivery. The method of delivery will be chosen for the particular agent and use. Parameters that affect delivery, as is known in the art, can include, inter alia, the cell type affected and cellular location. In some embodiments, “contacting” includes administering the agent to an individual. In some embodiments, “contacting” refers to exposing a cell or an environment in which the cell is located to one or more presently disclosed agents.

The present disclosure contemplates the use of any composition and/or agent that is capable of interfering with binding between the RNA transcribed from at least one regulatory element and the transcription factor itself. In some embodiments, modulating expression of the target gene occurs in vivo. In some embodiments, modulating expression of the target gene comprises administering to a subject an effective amount of a composition which interferes with binding between RNA transcribed from at least one regulatory element and the transcription factor.

The presently disclosed subject matter contemplates modulating expression (e.g., increasing and/or decreasing transcription) in cells, tissues, and subjects. In some embodiments, the cell or tissue includes one of the following: mammalian cell, e.g., human cell; fetal cell; embryonic stem cell or embryonic stem cell-like cell, e.g., cell from the umbilical vein, e.g., endothelial cell from the umbilical vein; muscle, e.g., myotube, fetal muscle; blood cell, e.g., cancerous blood cell, fetal blood cell, monocyte; B cell, e.g., Pro-B cell; brain, e.g., astrocyte cell, angular gyrus of the brain, anterior caudate of the brain, cingulate gyrus of the brain, hippocampus of the brain, inferior temporal lobe of the brain, middle frontal lobe of the brain, brain cancer cell; T cell, e.g., naive T cell, memory T cell; CD4 positive cell; CD25 positive cell; CD45RA positive cell; CD45RO positive cell; IL-17 positive cell; a cell that is stimulated with PMA; Th cell; Th17 cell; CD255 positive cell; CD127 positive cell; CD8 positive cell; CD34 positive cell; duodenum, e.g., smooth muscle tissue of the duodenum; skeletal muscle tissue; myoblast; stomach, e.g., smooth muscle tissue of the stomach, e.g., gastric cell; CD3 positive cell; CD14 positive cell; CD19 positive cell; CD20 positive cell; CD34 positive cell; CD56 positive cell; prostate, e.g., prostate cancer; colon, e.g., colorectal cancer cell; crypt cell, e.g., colon crypt cell; intestine, e.g., large intestine; e.g., fetal intestine; bone, e.g., osteoblast; pancreas, e.g., pancreatic cancer; adipose tissue; adrenal gland; bladder; esophagus; heart, e.g., left ventricle, right ventricle, left atrium, right atrium, aorta; lung, e.g., lung cancer cell; skin, e.g., fibroblast cell; ovary; psoas muscle; sigmoid colon; small intestine; spleen; thymus, e.g., fetal thymus; breast, e.g., breast cancer; cervix, e.g., cervical cancer; mammary epithelium; liver, e.g., liver cancer; DND41 cell; GM12878 cell; H1 cell; H2171 cell; HCC1954 cell; HCT-116 cell; HeLa cell; HepG2 cell; HMEC cell; HSMM tube cell; HUVEC cell; IMR90 cell; Jurkat cell; K562 cell; LNCaP cell; MCF-7 cell; MM1S cell; NHLF cell; NHDF-Ad cell; RPMI-8402 cell; U87 cell; VACO 9M cell; VACO 400 cell; or VACO 503 cell. In some embodiments, the cell is selected from the group consisting of adipocytes (e.g., white fat cell or brown fat cell), cardiac myocytes, chondrocytes, endothelial cells, exocrine gland cells, fibroblasts, glial cells, hepatocytes, keratinocytes, macrophages, monocytes, melanocytes, neurons, neutrophils, osteoblasts, osteoclasts, pancreatic islet cells (e.g., a beta cell), skeletal myocytes, smooth muscle cells, B cells, plasma cells, T cells (e.g., regulatory, cytotoxic, helper), and dendritic cells.

In some embodiments, the methods, compositions and/or agents disclosed herein can be used to modulate levels of expression of cell type specific genes and/or cell state specific genes. Modulating levels of expression of cell type specific genes and/or cell state specific genes may be useful, for example, to change a cell type from a cell of a first type to a cell of a second type (e.g., directed differentiation of a pluripotent cell to a desired cell type, reprogramming of a somatic cell, e.g., to a pluripotent state, or transdifferentiation of a somatic cell, e.g., to a different somatic cell) or to change a cell from one state to another state (e.g., shifting a cell from an “abnormal” state towards a more “normal” state, shifting a cell from a “disease-associated” state towards a more “healthy” state, shifting the cells from an “activated” state to a “resting” or “non-activated” state, etc.).

A cell type specific gene is typically expressed selectively in one or a small number of cells types relative to expression in many or most other cell types. One of skill in the art will be aware of numerous genes that are considered cell type specific. A cell type specific gene need not be expressed only in a single cell type but may be expressed in one or several, e.g., up to about 5, or about 10 different cell types out of the approximately 200 commonly recognized (e.g., in standard histology textbooks) and/or most abundant cell types in an adult vertebrate, e.g., mammal, e.g., human. In some embodiments, a cell type specific gene is one whose expression level can be used to distinguish a cell, e.g., a cell as disclosed herein, such as a cell of one of the following types from cells of the other cell types: adipocyte (e.g., white fat cell or brown fat cell), cardiac myocyte, chondrocyte, endothelial cell, exocrine gland cell, fibroblast, glial cell, hepatocyte, keratinocyte, macrophage, monocyte, melanocyte, neuron, neutrophil, osteoblast, osteoclast, pancreatic islet cell (e.g., a beta cell), skeletal myocyte, smooth muscle cell, B cell, plasma cell, T cell (e.g., regulatory, cytotoxic, helper), or dendritic cell. In some embodiments a cell type specific gene is lineage specific, e.g., it is specific to a particular lineage (e.g., hematopoietic, neural, muscle, etc.) In some embodiments, a cell-type specific gene is a gene that is more highly expressed in a given cell type than in most (e.g., at least 80%, at least 90%) or all other cell types. Thus specificity may relate to level of expression, e.g., a gene that is widely expressed at low levels but is highly expressed in certain cell types could be considered cell type specific to those cell types in which it is highly expressed. It will be understood that expression can be normalized based on total mRNA expression (optionally including miRNA transcripts, long non-coding RNA transcripts, and/or other RNA transcripts) and/or based on expression of a housekeeping gene in a cell. In some embodiments, a gene is considered cell type specific for a particular cell type if it is expressed at levels at least 2, 5, or at least 10-fold greater in that cell than it is, on average, in at least 25%, at least 50%, at least 75%, at least 90% or more of the cell types of an adult of that species, or in a representative set of cell types. One of skill in the art will be aware of databases containing expression data for various cell types, which may be used to select cell type specific genes. In some embodiments a cell type specific gene is a transcription factor.

In some aspects, modulating binding between an RNA transcribed from at least one regulatory element of a target gene and a transcription factor which binds to both the RNA and the regulatory element shifts a cell from an “abnormal” state towards a more “normal” state.

In some embodiments, modulating binding between an RNA transcribed from at least one regulatory element of a target gene and a transcription factor which binds to both the RNA and the regulatory element shifts a cell from a “disease-associated” state towards a state that is not associated with disease. A “disease-associated state” is a state that is typically found in subjects suffering from a disease (and usually not found in subjects not suffering from the disease) and/or a state in which the cell is abnormal, unhealthy, or contributing to a disease.

In some aspects, modulating binding between an RNA transcribed from at least one regulatory element of a target gene and a transcription factor which binds to both the RNA and the regulatory element reprograms a somatic cell, e.g., to a pluripotent state. In some aspects, modulating binding between an RNA transcribed from at least one regulatory element of a target gene and a transcription factor which binds to both the RNA and the regulatory element can be used to direct differentiation of a cell, e.g., from a pluripotent state to a cell of a desired cell type. In some embodiments, the methods, compositions and agents herein are of use to reprogram a somatic cell, e.g., to a pluripotent state. In some embodiments the methods, compositions and agents are of use to reprogram a somatic cell of a first cell type into a different cell type. In some embodiments, the methods, compositions and agents herein are of use to differentiate a pluripotent cell to a desired cell type.

In some aspects, modulating binding between an RNA transcribed from at least one regulatory element of a target gene and a transcription factor which binds to both the RNA and the regulatory element shifts a cell from an activated state to a resting or non-activated state. In some aspects, modulating binding between an RNA transcribed from at least one regulatory element of a target gene and a transcription factor which binds to both the RNA and the regulatory element shifts a cell from a non-activated state or resting state to an activated state. Another example of cell state is “activated” state as compared with “resting” or “non-activated” state. Many cell types in the body have the capacity to respond to a stimulus by modifying their state to an activated state. The particular alterations in state may differ depending on the cell type and/or the particular stimulus. A stimulus could be any biological, chemical, or physical agent to which a cell may be exposed. A stimulus could originate outside an organism (e.g., a pathogen such as virus, bacteria, or fungi (or a component or product thereof such as a protein, carbohydrate, or nucleic acid, cell wall constituent such as bacterial lipopolysaccharide, and the like) or may be internally generated (e.g., a cytokine, chemokine, growth factor, or hormone produced by other cells in the body or by the cell itself). For example, stimuli can include interleukins, interferons, or TNF alpha. Immune system cells, for example, can become activated upon encountering foreign (or in some instances host cell) molecules. Cells of the adaptive immune system can become activated upon encountering a cognate antigen (e.g., containing an epitope specifically recognized by the cell's T cell or B cell receptor) and, optionally, appropriate co-stimulating signals. Activation can result in changes in gene expression, production and/or secretion of molecules (e.g., cytokines, inflammatory mediators), and a variety of other changes that, for example, aid in defense against pathogens but can, e.g., if excessive, prolonged, or directed against host cells or host cell molecules, contribute to diseases. Fibroblasts are another cell type that can become activated in response to a variety of stimuli (e.g., injury (e.g., trauma, surgery), exposure to certain compounds including a variety of pharmacological agents, radiation, etc.) leading them, for example, to secrete extracellular matrix components. In the case of response to injury, such ECM components can contribute to wound healing. However, fibroblast activation, e.g., if prolonged, inappropriate, or excessive, can lead to a range of fibrotic conditions affecting diverse tissues and organs (e.g., heart, kidney, liver, intestine, blood vessels, skin) and/or contribute to cancer. The presence of abnormally large amounts of ECM components can result in decreased tissue and organ function, e.g., by increasing stiffness and/or disrupting normal structure and connectivity.

In some embodiments, the composition comprises an agent which binds to the transcription factor in a manner that prevents the transcription factor from binding to the RNA transcribed from the at least one regulatory element. In some embodiments, the agent binds to the transcription factor at the same site that the RNA transcribed from at least one regulatory element would bind to the transcription factor. In some embodiments, the agent binds to at least a portion of the same site that the RNA transcribed from at least one regulatory element would bind to the transcription factor (i.e., the agent binds to one or more amino acids of the transcription factor binding site for the RNA transcribed from the at least one regulatory element, but does not bind to all of the amino acids of such site). In some embodiments, the agent binds to the transcription factor in proximity to where RNA transcribed from at least one regulatory element binds to the transcription factor, but the agent masks the RNA binding site so the RNA can no longer bind to the transcription factor. In some embodiments, the agent binds to the transcription factor away from where the RNA transcribed from at least one regulatory element binds to the transcription factor, but the agent causes the transcription factor to change its conformation such that the RNA transcribed from at least one regulatory element can no longer bind to the transcription factor. In some embodiments, binding of the agent to the transcription factor affects another protein or cofactor that interacts with the transcription factor and the other protein or cofactor inhibits the RNA transcribed from at least one regulatory element from binding to the transcription factor.

In some embodiments, the agent does not bind to the at least one regulatory element. In some embodiments, the agent does not bind to the transcription factor in proximity to the DNA binding domain of the transcription factor or in a way that interferes with the DNA binding domain of the transcription factor. A person with skill in the art knows standard techniques for determining whether an agent binds or interferes with the DNA binding domain of the transcription factor. For example, electrophoretic mobility shift assays (EMSAs) can be performed with and without the agent to determine if the transcription factor-DNA interaction still occurs or is inhibited. For example, an EMSA can be performed after incubating the DNA sequence comprising the DNA binding site of the transcription factor with the transcription factor and a test agent.

In some embodiments, the agent which interferes with binding between the RNA and the transcription factor is selected from the group consisting of small molecules, saccharides, peptides, proteins, peptidomimetics, nucleic acids, an extract made from biological materials selected from the group consisting of bacteria, plants, fungi, animal cells, and animal tissues, and any combination thereof. As used herein, small molecules refers to compounds having a molecular weight of less than about 2 kilodaltons. In some embodiments, the small molecule has a molecular weight of less than about 1000 daltons. In some embodiments, the small molecule has a molecular weight of less than about 500 daltons.

The presently disclosed subject matter contemplates the use of synthetic, chemically modified nucleic acid molecules. The synthetic, chemically modified nucleic acid molecules are useful in the treatment of any disease or condition that responds to modulation of gene expression or activity in a cell, tissue, or organism, and in particular are useful for modulating binding between RNA transcribed from regulatory elements occupied by transcription factors that bind to the transcribed RNA, as well as the regulatory elements. The synthetic, chemically modified nucleic acid molecules can be used to increase or decrease transcription of target genes.

Exemplary nucleic acids include ribonucleic acids (RNAs), deoxyribonucleic acids (DNAs), threose nucleic acids (TNAs), glycol nucleic acids (GNAs), peptide nucleic acids (PNAs), locked nucleic acids (LNAs) or a hybrid thereof (e.g., In some embodiments, the nucleic acids comprise short interfering nucleic acid (siNA), short interfering RNA (siRNA), double-stranded RNA (dsRNA), micro-RNA (miRNA), and short hairpin RNA (shRNA) molecules capable of mediating RNA interference (RNAi) against target nucleic acid sequences. In some embodiments, the nucleic acid comprises messenger RNA (mRNA). In some embodiments, the nucleic acids of the invention do not substantially induce an innate immune response of a cell into which the nucleic acid is introduced.

Various modifications to the structures of the nucleic acid can be made to enhance the utility of these molecules. Such modifications will enhance shelf-life, half-life in vitro, stability, and ease of introduction of such oligonucleotides to the target site, e.g., to enhance penetration of cellular membranes, and confer the ability to recognize and bind to targeted cells.

As used herein, “non-nucleotide” means any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine and therefore lacks a base at the 1′-position.

As used herein “nucleotide” as is as recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see, for example, Usman and McSwiggen, supra; Eckstein et al., International PCT Publication No. WO 92/07065; Usman et al., International PCT Publication No. WO 93/15187; Uhlman & Peyman, supra, all are hereby incorporated by reference herein). There are several examples of modified nucleic acid bases known in the art as summarized by Limbach et al., 1994, Nucleic Acids Res. 22, 2183. Some of the non-limiting examples of base modifications that can be introduced into nucleic acid molecules include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents.

As used herein “abasic” means sugar moieties lacking a base or having other chemical groups in place of a base at the 1′ position, see for example Adamic et al., U.S. Pat. No. 5,998,203.

As used herein “unmodified nucleoside” means one of the bases adenine, cytosine, guanine, thymine, or uracil joined to the 1′ carbon of .beta.-D-ribo-furanose.

As used herein, “modified nucleoside” means any nucleotide base which contains a modification in the chemical structure of an unmodified nucleotide base, sugar and/or phosphate.

In some embodiments, the nucleic acids of the presently disclosed subject matter include phosphate backbone modifications comprising one or more phosphorothioate, phosphonoacetate, and/or thiophosphonoacetate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications, see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39.

The nucleic acids disclosed herein (e.g., synthetic RNAs, including modified mRNAs) can be conjugated to non-nucleic acid molecules. In some embodiments, the nucleic acids disclosed herein (e.g., synthetic RNAs) are conjugated to (or otherwise physically associated with) a moiety that promotes cellular uptake, nuclear entry, and/or nuclear retention. For example, the present disclosure contemplates conjugates of peptide transport moieties and the nucleic acids. In some embodiments, the nucleic acid is conjugated to a peptide transporter moiety, for example a cell-penetrating peptide transport moiety, which is effective to enhance transport of the oligomer into cells. For example, in some embodiments the peptide transporter moiety is an arginine-rich peptide. In further embodiments, the transport moiety is attached to either the 5′ or 3′ terminus of the oligomer. When such peptide is conjugated to either termini, the opposite termini is then available for further conjugation to a modified terminal group as described herein. Peptide transport moieties are generally effective to enhance cell penetration of the nucleic acids. In some embodiments, a glycine (G) or proline (P) amino acid subunit is included between the nucleic acid and the remainder of the peptide transport moiety (e.g., at the carboxy or amino terminus of the carrier peptide) to reduces the toxicity of the conjugate, while maintaining or improving efficacy relative to conjugates with different linkages between the peptide transport moiety and nucleic acid.

A reporter moiety, such as fluorescein or a radiolabeled group, may be attached to nucleic acids disclosed herein for purposes of detection. Alternatively, the reporter label attached to the oligomer may be a ligand, such as an antigen or biotin, capable of binding a labeled antibody or streptavidin. In selecting a moiety for attachment or modification of a nucleic acid molecule, it is generally of course desirable to select chemical compounds of groups that are biocompatible and likely to be tolerated by a subject without undesirable side effects.

In some embodiments, the agent comprises a decoy RNA. As used herein, the term “decoy RNA” refers to an RNA which binds to either the transcription factor or the nascent RNA transcribed from the at least one regulatory element in a manner that interferes with the interaction between the nascent transcribed RNA and the transcription factor. For example, a decoy RNA can bind to the transcription factor in a manner that outcompetes the nascent RNA transcribed from the at least one regulatory element for binding to the transcription factor. In some embodiments, the decoy RNA binds to the transcription factor in a manner that outcompetes the nascent RNA transcribed from the at least one regulatory element for binding to the transcription factor in the absence of directly competing with binding of the transcription factor to the at least one regulatory sequence.

In some embodiments, the decoy RNA comprises a synthetic RNA having a nucleotide sequence that is homologous to the RNA transcribed from the at least one regulatory element. As used herein, the term “synthetic RNA” refers to an RNA molecule that can be generated by in vitro transcription, by direct chemical synthesis or an RNA molecule that is produced in a genetically engineered cell, such as in a bacterial cell, for e.g., in an E. coli cell, but is not produced by that type of cell if it is not genetically engineered. In some contexts, the synthetic RNA molecule contains at least one non-naturally occurring modification compared to its counterpart naturally occurring RNA. As used herein, a synthetic RNA that includes “at least one modification” contains such at least one non-naturally occurring modification. It should appreciate that nucleic acids of use herein that contain at least one modification may, in some embodiments, contain other naturally occurring modifications.

Methods for generating DNA templates for in vitro transcription are well known to those of skill in the art using standard molecular cloning techniques. Approaches to the assembly of DNA templates that do not rely upon the presence of restriction endonuclease cleavage sites are also envisioned, e.g., splint-mediated ligation. The transcribed, synthetic RNA can be modified further post-transcription, e.g., by adding a cap or other functional group. In an aspect, a synthetic RNA comprises a 5′ and/or a 3′-cap structure. Synthetic RNA can be single stranded (e.g., ssRNA) or double stranded (e.g., dsRNA). The 5′ and/or 3′-cap structure can be on only the sense strand, the antisense strand, or both strands. By “cap structure” is meant chemical modifications, which have been incorporated at either terminus of the oligonucleotide (see, for example, Adamic et al., U.S. Pat. No. 5,998,203, incorporated by reference herein). These terminal modifications protect the nucleic acid molecule from exonuclease degradation, and can help in delivery and/or localization within a cell. The cap can be present at the 5′-terminus (5′-cap) or at the 3′-terminal (3′-cap) or can be present on both termini.

Non-limiting examples of the 5′-cap include, but are not limited to, glyceryl, inverted deoxy abasic residue (moiety); 4′,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

Non-limiting examples of the 3′-cap include, but are not limited to, glyceryl, inverted deoxy abasic residue (moiety), 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

The synthetic RNA may comprise at least one modified nucleoside, such as pseudouridine, mSU, s2U, m6A, and mSC, N1-methylguanosine, N1-methyladenosine, N7-methylguanosine, 2′-)-methyluridine, and 2′-O-methylcytidine. Polymerases that accept modified nucleosides are known to those of skill in the art. Modified polymerases can be used to generate synthetic, modified RNAs. Thus, for example, a polymerase that tolerates or accepts a particular modified nucleoside as a substrate can be used to generate a synthetic, modified RNA including that modified nucleoside.

In some embodiments, the synthetic RNA provokes a reduced (or absent) innate immune response in vivo or reduced interferon response in vivo by the transfected tissue or cell population. mRNA produced in eukaryotic cells, e.g., mammalian or human cells, is heavily modified, the modifications permitting the cell to detect RNA not produced by that cell. The cell responds by shutting down translation or otherwise initiating an innate immune or interferon response. Thus, to the extent that an exogenously added RNA can be modified to mimic the modifications occurring in the endogenous RNAs produced by a target cell, the exogenous RNA can avoid at least part of the target cell's defense against foreign nucleic acids. Thus, in some embodiments, synthetic RNAs include in vitro transcribed RNAs including modifications as found in eukaryotic/mammalian/human RNA in vivo. Other modifications that mimic such naturally occurring modifications can also be helpful in producing a synthetic RNA molecule that will be tolerated by a cell.

In some embodiments, the synthetic RNA is at least 81% identical to RNA transcribed from the at least one regulatory element. In some embodiments, the synthetic RNA is at least 82% identical to RNA transcribed from the at least one regulatory element. In some embodiments, the synthetic RNA is at least 83% identical to RNA transcribed from the at least one regulatory element. In some embodiments, the synthetic RNA is at least 84% identical to RNA transcribed from the at least one regulatory element. In some embodiments, the synthetic RNA is at least 85% identical to RNA transcribed from the at least one regulatory element. In some embodiments, the synthetic RNA is at least 86% identical to RNA transcribed from the at least one regulatory element. In some embodiments, the synthetic RNA is at least 87% identical to RNA transcribed from the at least one regulatory element. In some embodiments, the synthetic RNA is at least 88% identical to RNA transcribed from the at least one regulatory element. In some embodiments, the synthetic RNA is at least 89% identical to RNA transcribed from the at least one regulatory element. In some embodiments, the synthetic RNA is at least 90% identical to RNA transcribed from the at least one regulatory element. In some embodiments, the synthetic RNA is at least 91% identical to RNA transcribed from the at least one regulatory element. In some embodiments, the synthetic RNA is at least 92% identical to RNA transcribed from the at least one regulatory element. In some embodiments, the synthetic RNA is at least 93% identical to RNA transcribed from the at least one regulatory element. In some embodiments, the synthetic RNA is at least 94% identical to RNA transcribed from the at least one regulatory element. In some embodiments, the synthetic RNA is at least 95% identical to RNA transcribed from the at least one regulatory element. In some embodiments, the synthetic RNA is at least 96% identical to RNA transcribed from the at least one regulatory element. In some embodiments, the synthetic RNA is at least 96% identical to RNA transcribed from the at least one regulatory element. In some embodiments, the synthetic RNA is at least 97% identical to RNA transcribed from the at least one regulatory element. In some embodiments, the synthetic RNA is at least 98% identical to RNA transcribed from the at least one regulatory element. In some embodiments, the synthetic RNA is at least 99% identical to RNA transcribed from the at least one regulatory element. In some embodiments, the synthetic RNA comprises at least one, two, three, four, five, six, seven, eight, nine, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 175, 200, 225, 250, or more, mismatched nucleotides as compared to the RNA transcribed from the at least one regulatory element.

In some embodiments, the synthetic RNA is at least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% identical to RNA transcribed from the at least one regulatory element and contains at least one, two, three, four, five, six, seven, eight, nine, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 175, 200, 225, 250, or more, mismatched nucleotides as compared to the RNA transcribed from the at least one regulatory element.

In some embodiments, the synthetic RNA consists of, consists essentially of a nucleotide sequence that is at least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% identical to RNA transcribed from the at least one regulatory element and contains at least one, two, three, four, five, six, seven, eight, nine, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 175, 200, 225, 250, or more, mismatched nucleotides as compared to the RNA transcribed from the at least one regulatory element, and comprises at least one modification.

In some embodiments, the synthetic RNA consists of, consists essentially of, or comprises a nucleotide sequence that comprises an RNA binding site for the transcription factor.

In some embodiments, the synthetic RNA consists of, consists essentially of, or comprises a nucleotide sequence that is at least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% identical to the transcription factor binding site in the RNA transcribed from the at least one regulatory element and contains at least one, two, three, four, five, six, seven, eight, nine, or 10, 11, 12, 13, 14, 15, 16, 17, 18, 19, 20, 25, 30, or more, mismatched nucleotides as compared to the transcription factor binding site in the RNA transcribed from the at least one regulatory element.

In some embodiments, the synthetic RNA consists of, consists essentially of, or comprises a nucleotide sequence that is at least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% identical to the transcription factor binding site in the RNA transcribed from the at least one regulatory element and contains at least one, two, three, four, five, six, seven, eight, nine, or 10, 11, 12, 13, 14, 15, 16, 17, 18, 19, 20, 25, 30, or more, mismatched nucleotides as compared to the transcription factor binding site in the RNA transcribed from the at least one regulatory element, and comprises at least one modification.

In some embodiments, the synthetic RNA comprises a length of between 10 nucleotides and 300 nucleotides. In some embodiments, the synthetic RNA comprises a length of between 10 nucleotides and 300 nucleotides and contains at least 1, at least 2, at least 3, at least 4, at least 5, at least 7, at least 8, or at least 9, or at least 10, or more, mismatched nucleotides as compared to the transcription factor binding site of the RNA transcribed from the at least one regulatory element.

In some embodiments, the synthetic RNA comprises a length of between 10 nucleotides and 300 nucleotides and contains at least 1, at least 2, at least 3, at least 4, at least 5, at least 7, at least 8, or at least 9, or at least 10, or more, mismatched nucleotides as compared to the transcription factor binding site in the RNA transcribed from at least one regulatory element occupied by a transcription factor selected from the group consisting of YY1, Krueppel-like factor 4 (KLF4), Ronin (Thap11), RE1-silencing transcription factor (REST), PR domain zinc finger protein 14 (PRDM14), CCCTC-binding factor (CTCF), p53, Signal transducer and activator of transcription 1 (STAT1), TLS/FUS, BRCA1, DLX2, ESR1, FUS, KIN, KU, NACA, NCL, NFKB1, NFYA, NR3C1, RARA, RUNX1, SOX2, TCF7, and TP53. In some embodiments, the transcription factor is selected from the group consisting of Yin-Yang 1 (YY1), Krueppel-like factor 4 (KLF4), Ronin (Thap11), RE1-silencing transcription factor (REST), PR domain zinc finger protein 14 (PRDM14), CCCTC-binding factor (CTCF), Signal transducer and activator of transcription 1 (STAT1), and TLS/FUS, BRCA1, DLX2, ESR1, KIN, KU, NACA, NCL, NFKB1, NFYA, NR3C1, RARA, RUNX1, SOX2, TCF7, and TP53 (p53).

In some embodiments, the synthetic RNA comprises a length of between 30 and 60 nucleotides and binds to a transcription factor that occupies at least one regulatory element and binds to RNA transcribed from the at least one regulatory element, wherein the transcription factor is selected from the group consisting of YY1, Krueppel-like factor 4 (KLF4), Ronin (Thap11), RE1-silencing transcription factor (REST), PR domain zinc finger protein 14 (PRDM14), CCCTC-binding factor (CTCF), p53, Signal transducer and activator of transcription 1 (STAT1), TLS/FUS, BRCA1, DLX2, ESR1, FUS, KIN, KU, NACA, NCL, NFKB1, NFYA, NR3C1, RARA, RUNX1, SOX2, TCF7, and TP53. In some embodiments, the transcription factor is selected from the group consisting of Yin-Yang 1 (YY1), Krueppel-like factor 4 (KLF4), Ronin (Thap11), RE1-silencing transcription factor (REST), PR domain zinc finger protein 14 (PRDM14), CCCTC-binding factor (CTCF), Signal transducer and activator of transcription 1 (STAT1), and TLS/FUS, BRCA1, DLX2, ESR1, KIN, KU, NACA, NCL, NFKB1, NFYA, NR3C1, RARA, RUNX1, SOX2, TCF7, and TP53 (p53).

In some embodiments, the synthetic RNAs (e.g., decoy RNA) comprise a sequence having a length that is sufficient to target a unique sequence in the transcriptome (e.g., at least 10 nucleotides. In some embodiments, the decoy RNA comprises a sequence having a length that is therapeutically effective (e.g., a length less than 300, e.g., less than 200, e.g., preferably less than about 100 nucleotides). In some embodiments, the synthetic RNAs comprise a sequence having a length of between 12 and 50 nucleotides.

In some embodiments, the presently disclose subject matter contemplates utilizing at least 2, at least 3, at least 4, at least 5, or more synthetic RNAs targeting the same nascent RNA transcribed from the at least one regulatory element but in different regions. In some embodiments, at least 2, at least 3, at least 4, at least 5, or more synthetic RNAs targeting the same nascent RNA transcribed from the at least one regulatory element in different regions each comprise a length of between 10 and 300 nucleotides. In some embodiments, such synthetic RNAs each comprise a length of between about 10 an d100 nucleotides. In some embodiments, such synthetic RNAs each comprise a length of between 12 and 50 nucleotides. In some embodiments, such synthetic RNAs each comprise a length of between 15 and 30 nucleotides. In some embodiments, such synthetic RNAs each comprise a length of about 20, about 21, about 22, about 23, about 24, about 25, about 26, about 27, about 28, or about 29 nucleotides.

Each of such synthetic RNAs can include at least one modification. In some embodiments, the synthetic RNA comprises a length of between 30 and 60 nucleotides. In some embodiments, the synthetic RNA comprises a length of 20 nucleotidesnucleotides. In some embodiments, the synthetic RNA comprises a length of 21 nucleotidesnucleotides. In some embodiments, the synthetic RNA comprises a length of 22 nucleotidesnucleotides. In some embodiments, the synthetic RNA comprises a length of 23 nucleotidesnucleotides. In some embodiments, the synthetic RNA comprises a length of 24 nucleotides. In some embodiments, the synthetic RNA comprises a length of 25 nucleotides. In some embodiments, the synthetic RNA comprises a length of 26 nucleotides. In some embodiments, the synthetic RNA comprises a length of 27 nucleotides. In some embodiments, the synthetic RNA comprises a length of 28 nucleotides. In some embodiments, the synthetic RNA comprises a length of 29 nucleotides. In some embodiments, the synthetic RNA comprises a length of 30 nucleotides. In some embodiments, the synthetic RNA comprises a length of 35 nucleotides. In some embodiments, the synthetic RNA comprises a length of 40 nucleotides. In some embodiments, the synthetic RNA comprises a length of 45 nucleotides. In some embodiments, the synthetic RNA comprises a length of 50 nucleotides. In some embodiments, the synthetic RNA comprises a length of 55 nucleotides. In some embodiments, the synthetic RNA comprises a length of 60 nucleotides.

In some embodiments, the synthetic RNA comprises a length of 20 nucleotides and contains at least one modification. In some embodiments, the synthetic RNA comprises a length of 21 nucleotides and contains at least one modification. In some embodiments, the synthetic RNA comprises a length of 22 nucleotides and contains at least one modification. In some embodiments, the synthetic RNA comprises a length of 23 nucleotides and contains at least one modification. In some embodiments, the synthetic RNA comprises a length of 24 nucleotides and contains at least one modification. In some embodiments, the synthetic RNA comprises a length of 25 nucleotides and contains at least one modification. In some embodiments, the synthetic RNA comprises a length of 26 nucleotides and contains at least one modification. In some embodiments, the synthetic RNA comprises a length of 27 nucleotides and contains at least one modification. In some embodiments, the synthetic RNA comprises a length of 28 nucleotides and contains at least one modification. In some embodiments, the synthetic RNA comprises a length of 29 nucleotides and contains at least one modification. In some embodiments, the synthetic RNA comprises a length of 30 nucleotides and contains at least one modification. In some embodiments, the synthetic RNA comprises a length of 35 nucleotides and contains at least one modification. In some embodiments, the synthetic RNA comprises a length of 40 nucleotides and contains at least one modification. In some embodiments, the synthetic RNA comprises a length of 45 nucleotides and contains at least one modification. In some embodiments, the synthetic RNA comprises a length of 50 nucleotides and contains at least one modification. In some embodiments, the synthetic RNA comprises a length of 55 nucleotides and contains at least one modification. In some embodiments, the synthetic RNA comprises a length of 60 nucleotides and contains at least one modification.

In some embodiments, the transcription factor is YY1 and the synthetic RNA comprises a nucleotide sequence that is homologous to the RNA transcribed from the at least one regulatory element in a chromosomal region identified in Table 5 or Table 6, each of which are disclosed in U.S. Provisional Application No. 62/248,119, filed Oct. 29, 2015, which is incorporated herein by reference in its entirety.

In some embodiments, the synthetic RNA consists of, consists essentially of, or comprises a nucleotide sequence that is at least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% identical to RNA transcribed from at least one regulatory element in a chromosomal region identified in Table 5 or Table 6, each of which are disclosed in U.S. Provisional Application No. 62/248,119, filed Oct. 29, 2015, which is incorporated herein by reference in its entirety.

In some embodiments, the synthetic RNA consists of, consists essentially of, or comprises a nucleotide sequence that is at least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% identical to RNA transcribed from at least one regulatory element in a chromosomal region identified in Table 5 or Table 6, each of which are disclosed in U.S. Provisional Application No. 62/248,119, filed Oct. 29, 2015, which is incorporated herein by reference in its entirety, and contains at least one, two, three, four, five, six, seven, eight, nine, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 175, 200, 225, 250, or more, mismatched nucleotides as compared to the RNA transcribed from the at least one regulatory element in the chromosomal region identified in Table 5 or Table 6, each of which are disclosed in U.S. Provisional Application No. 62/248,119, filed Oct. 29, 2015, which is incorporated herein by reference in its entirety.

In some embodiments, the synthetic RNA consists of, consists essentially of, or comprises a nucleotide sequence that is at least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% identical to the transcription factor binding site (in the RNA transcribed from the at least one regulatory element in a chromosomal region identified in Table 5 or Table 6, each of which are disclosed in U.S. Provisional Application No. 62/248,119, filed Oct. 29, 2015, which is incorporated herein by reference in its entirety, and contains at least one, two, three, four, five, six, seven, eight, nine, or 10, 11, 12, 13, 14, 15, 16, 17, 18, 19, 20, 25, 30, or more, mismatched nucleotides as compared to the RNA transcribed from the at least one regulatory element, and comprises at least one modification.

The presently disclosed subject matter also contemplates synthetic RNA consisting of, consisting essentially of, or comprising nucleotide sequences that are at least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% identical to RNA transcribed from at least one regulatory element occupied by a transcription factor selected from the group consisting of Krueppel-like factor 4 (KLF4), Ronin (Thap11), RE1-silencing transcription factor (REST), PR domain zinc finger protein 14 (PRDM14), CCCTC-binding factor (CTCF), p53, Signal transducer and activator of transcription 1 (STAT1), TLS/FUS, BRCA1, DLX2, ESR1, FUS, KIN, KU, NACA, NCL, NFKB1, NFYA, NR3C1, RARA, RUNX1, SOX2, TCF7, and TP53. In some embodiments, the transcription factor is selected from the group consisting of Yin-Yang 1 (YY1), Krueppel-like factor 4 (KLF4), Ronin (Thap11), RE1-silencing transcription factor (REST), PR domain zinc finger protein 14 (PRDM14), CCCTC-binding factor (CTCF), Signal transducer and activator of transcription 1 (STAT1), and TLS/FUS, BRCA1, DLX2, ESR1, KIN, KU, NACA, NCL, NFKB1, NFYA, NR3C1, RARA, RUNX1, SOX2, TCF7, and TP53 (p53).

In some embodiments, the synthetic RNA consists of, consists essentially of, or comprises a nucleotide sequence that is least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% identical to RNA transcribed from at least one regulatory element occupied by a transcription factor selected from the group consisting of Krueppel-like factor 4 (KLF4), Ronin (Thap11), RE1-silencing transcription factor (REST), PR domain zinc finger protein 14 (PRDM14), CCCTC-binding factor (CTCF), p53, Signal transducer and activator of transcription 1 (STAT1), TLS/FUS, BRCA1, DLX2, ESR1, FUS, KIN, KU, NACA, NCL, NFKB1, NFYA, NR3C1, RARA, RUNX1, SOX2, TCF7, and TP53. In some embodiments, the transcription factor is selected from the group consisting of Yin-Yang 1 (YY1), Krueppel-like factor 4 (KLF4), Ronin (Thap11), RE1-silencing transcription factor (REST), PR domain zinc finger protein 14 (PRDM14), CCCTC-binding factor (CTCF), Signal transducer and activator of transcription 1 (STAT1), and TLS/FUS, BRCA1, DLX2, ESR1, KIN, KU, NACA, NCL, NFKB1, NFYA, NR3C1, RARA, RUNX1, SOX2, TCF7, and TP53 (p53) and contains at least one, two, three, four, five, six, seven, eight, nine, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 175, 200, 225, 250, or more, mismatched nucleotides as compared to the RNA transcribed from the at least one regulatory element.

In some embodiments, the synthetic RNA consists of, consists essentially of, or comprises a nucleotide sequence that is least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% identical to RNA transcribed from at least one regulatory element occupied by transcription factors

Krueppel-like factor 4 (KLF4), Ronin (Thap11), RE1-silencing transcription factor (REST), PR domain zinc finger protein 14 (PRDM14), CCCTC-binding factor (CTCF), p53, Signal transducer and activator of transcription 1 (STAT1), TLS/FUS, BRCA1, DLX2, ESR1, FUS, KIN, KU, NACA, NCL, NFKB1, NFYA, NR3C1, RARA, RUNX1, SOX2, TCF7, and TP53. In some embodiments, the transcription factor is selected from the group consisting of Yin-Yang 1 (YY1), Krueppel-like factor 4 (KLF4), Ronin (Thap11), RE1-silencing transcription factor (REST), PR domain zinc finger protein 14 (PRDM14), CCCTC-binding factor (CTCF), Signal transducer and activator of transcription 1 (STAT1), and TLS/FUS, BRCA1, DLX2, ESR1, KIN, KU, NACA, NCL, NFKB1, NFYA, NR3C1, RARA, RUNX1, SOX2, TCF7, and TP53 (p53) and contains at least one, two, three, four, five, six, seven, eight, nine, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 175, 200, 225, 250, or more, mismatched nucleotides as compared to the RNA transcribed from the at least one regulatory element, and comprises at least one modification.

The presently disclosed subject matter also contemplates synthetic RNA consisting of, consisting essentially of, or comprising nucleotide sequences that are at least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% identical to RNA transcribed from at least one regulatory element occupied by a transcription factor of interest in a cell type of interest within an organism of interest. For example, candidate transcription factors of interest can be identified as noted above, and the methods disclosed herein can be used to design suitable synthetic RNAs that are capable of binding to RNAs transcribed from regulatory elements of target genes regulated by such transcription factors. In some embodiments, such synthetic RNA contains at least one, two, three, four, five, six, seven, eight, nine, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 175, 200, 225, 250, or more, mismatched nucleotides as compared to the RNA transcribed from the at least one regulatory element.

In some embodiments, the decoy RNA binds to the nascent RNA transcribed from the at least one regulatory element in a manner that prevents the nascent RNA from binding to the transcription factor. In some embodiments, the decoy RNA comprises a synthetic RNA having a sequence that is complementary to the nascent RNA. In some embodiments, the decoy RNA comprises a synthetic RNA having a sequence that is complementary to at least a portion of the nascent RNA. In some embodiments, the decoy RNA comprises a synthetic RNA having a sequence that is complementary to the transcription factor binding site in the nascent RNA transcribed from the at least one regulatory element. In some embodiments, the decoy RNA comprises a synthetic RNA having a sequence that is complementary to at least a portion of the transcription factor binding site in the nascent RNA transcribed from the at least one regulatory element.

In some embodiments, the decoy RNA comprises a synthetic RNA having a length of between 10 and 300 nucleotides and a sequence that is complementary to at least a portion of the nascent RNA transcribed from the at least one regulatory element. In some embodiments, the decoy RNA comprises a synthetic RNA having a length of between 10 and 300 nucleotides and a sequence that is complementary to at least a portion of the transcription factor binding site in the nascent RNA transcribed from the at least one regulatory element. In some embodiments, the synthetic RNA has a length of between 10 and 300 nucleotides and has a sequence that is complementary to at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of a sequence of nascent RNA transcribed from the at least one regulatory element.

In some embodiments, the synthetic RNA has a length of between 30 and 60 nucleotides and has a sequence that is complementary to at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of a sequence of RNA transcribed from the at least one regulatory element. In some embodiments, the synthetic RNA has a length of between 30 and 60 nucleotides and contains at least one, two, three, four, five, six, seven, eight, nine, 10, 11, 12, 13, 14, 15, 16, 17, 18, 19, 20, 25, 30, 35, 40, 45, 50, 55, or more, nucleotides that are complementary to the nascent RNA transcribed from the at least one regulatory element.

In some embodiments, the transcription factor is YY1 and the synthetic RNA comprises a nucleotide sequence that is complementary to a sequence of RNA transcribed from at least one regulatory element in a chromosomal region identified in Table 5 or Table 6, each of which are disclosed in U.S. Provisional Application No. 62/248,119, filed Oct. 29, 2015, which is incorporated herein by reference in its entirety. In some embodiments, the synthetic RNA consists of, consists essentially of, or comprises a nucleotide sequence that complementary to at least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% of a sequence of RNA transcribed from at least one regulatory element in a chromosomal region identified in Table 5 or Table 6, each of which are disclosed in U.S. Provisional Application No. 62/248,119, filed Oct. 29, 2015, which is incorporated herein by reference in its entirety.

In some embodiments, the synthetic RNA consists of, consists essentially of, or comprises a nucleotide sequence that is complementary to at least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% of a sequence of RNA transcribed from at least one regulatory element in a chromosomal region identified in Table 5 or Table 6, each of which are disclosed in U.S. Provisional Application No. 62/248,119, filed Oct. 29, 2015, which is incorporated herein by reference in its entirety, and optionally contains at least one, two, three, four, five, six, seven, eight, nine, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 175, 200, 225, 250, or more, nucleotides that are not complementary to the RNA transcribed from the at least one regulatory element in the chromosomal region identified in Table 5 or Table 6, each of which are disclosed in U.S. Provisional Application No. 62/248,119, filed Oct. 29, 2015, which is incorporated herein by reference in its entirety.

In some embodiments, the synthetic RNA consists of, consists essentially of, or comprises a nucleotide sequence that is complementary to at least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% of the transcription factor binding sequence of nascent RNA transcribed from at least one regulatory element in a chromosomal region identified in Table 5 or Table 6, each of which are disclosed in U.S. Provisional Application No. 62/248,119, filed Oct. 29, 2015, which is incorporated herein by reference in its entirety, and optionally contains at least one, two, three, four, five, six, seven, eight, nine, or 10, 11, 12, 13, 14, 15, 16, 17, 18, 19, 20, 25, 30, or more, nucleotides that are not complementary to the RNA transcribed from the at least one regulatory element, and comprises at least one modification.

The presently disclosed subject matter also contemplates synthetic RNA consisting of, consisting essentially of, or comprising nucleotide sequences that are at least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% complementary to RNA transcribed from at least one regulatory element occupied by a transcription factor selected from the group consisting of

Krueppel-like factor 4 (KLF4), Ronin (Thap11), RE1-silencing transcription factor (REST), PR domain zinc finger protein 14 (PRDM14), CCCTC-binding factor (CTCF), p53, Signal transducer and activator of transcription 1 (STAT1), TLS/FUS, BRCA1, DLX2, ESR1, FUS, KIN, KU, NACA, NCL, NFKB1, NFYA, NR3C1, RARA, RUNX1, SOX2, TCF7, and TP53. In some embodiments, the transcription factor is selected from the group consisting of Yin-Yang 1 (YY1), Krueppel-like factor 4 (KLF4), Ronin (Thap11), RE1-silencing transcription factor (REST), PR domain zinc finger protein 14 (PRDM14), CCCTC-binding factor (CTCF), Signal transducer and activator of transcription 1 (STAT1), and TLS/FUS, BRCA1, DLX2, ESR1, KIN, KU, NACA, NCL, NFKB1, NFYA, NR3C1, RARA, RUNX1, SOX2, TCF7, and TP53 (p53).

In some embodiments, the synthetic RNA consists of, consists essentially of, or comprises a nucleotide sequence that is least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% complementary to RNA transcribed from at least one regulatory element occupied by a transcription factor selected from the group consisting of Krueppel-like factor 4 (KLF4), Ronin (Thap11), RE1-silencing transcription factor (REST), PR domain zinc finger protein 14 (PRDM14), CCCTC-binding factor (CTCF), p53, Signal transducer and activator of transcription 1 (STAT1), TLS/FUS, BRCA1, DLX2, ESR1, FUS, KIN, KU, NACA, NCL, NFKB1, NFYA, NR3C1, RARA, RUNX1, SOX2, TCF7, and TP53. In some embodiments, the transcription factor is selected from the group consisting of Yin-Yang 1 (YY1), Krueppel-like factor 4 (KLF4), Ronin (Thap11), RE1-silencing transcription factor (REST), PR domain zinc finger protein 14 (PRDM14), CCCTC-binding factor (CTCF), Signal transducer and activator of transcription 1 (STAT1), and TLS/FUS, BRCA1, DLX2, ESR1, KIN, KU, NACA, NCL, NFKB1, NFYA, NR3C1, RARA, RUNX1, SOX2, TCF7, and TP53 (p53) and optionally contains at least one, two, three, four, five, six, seven, eight, nine, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 175, 200, 225, 250, or more, nucleotides that are not complementary to the RNA transcribed from the at least one regulatory element.

In some embodiments, the synthetic RNA consists of, consists essentially of, or comprises a nucleotide sequence that is least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% complementary to RNA transcribed from at least one regulatory element occupied by transcription factors Krueppel-like factor 4 (KLF4), Ronin (Thap11), RE1-silencing transcription factor (REST), PR domain zinc finger protein 14 (PRDM14), CCCTC-binding factor (CTCF), p53, Signal transducer and activator of transcription 1 (STAT1), TLS/FUS, BRCA1, DLX2, ESR1, FUS, KIN, KU, NACA, NCL, NFKB1, NFYA, NR3C1, RARA, RUNX1, SOX2, TCF7, and TP53. In some embodiments, the transcription factor is selected from the group consisting of Yin-Yang 1 (YY1), Krueppel-like factor 4 (KLF4), Ronin (Thap11), RE1-silencing transcription factor (REST), PR domain zinc finger protein 14 (PRDM14), CCCTC-binding factor (CTCF), Signal transducer and activator of transcription 1 (STAT1), and TLS/FUS, BRCA1, DLX2, ESR1, KIN, KU, NACA, NCL, NFKB1, NFYA, NR3C1, RARA, RUNX1, SOX2, TCF7, and TP53 (p53) and optionally contains at least one, two, three, four, five, six, seven, eight, nine, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 175, 200, 225, 250, or more, nucleotides that are not complementary to the RNA transcribed from the at least one regulatory element, and comprises at least one modification.

The presently disclosed subject matter also contemplates synthetic RNA consisting of, consisting essentially of, or comprising nucleotide sequences that are at least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% complementary to nascent RNA transcribed from at least one regulatory element occupied by a transcription factor of interest in a cell type of interest within an organism of interest. For example, candidate transcription factors of interest can be identified as noted above, and the methods disclosed herein can be used to design suitable synthetic RNAs that are capable of binding to RNAs transcribed from regulatory elements of target genes regulated by such transcription factors. In some embodiments, such synthetic RNA optionally contains at least one, two, three, four, five, six, seven, eight, nine, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 175, 200, 225, 250, or more, nucleotides that are not complementary to the RNA transcribed from the at least one regulatory element.

In some embodiments, the agent (e.g., synthetic RNA) comprises a synthetic, modified messenger ribonucleic acid (mRNA) that encodes a peptide, polypeptide, or protein that is capable of interfering with binding between the RNA transcribed from at least one regulatory element and the transcription factor that binds to the RNA transcribed from at least one regulatory element and the at least one regulatory element. For example, synthetic, modified mRNAs (e.g., containing at least one modified nucleic acid as described herein) can be introduced into a cell, tissue, or subject (e.g., a mammalian cell, tissue, or subject, e.g., human cell, tissue, or subject), under conditions that the peptide, polypeptide, or protein is produced (e.g., translated) in the cell, tissue, or subject. In some embodiments, the peptide, polypeptide, or protein encoded by the mRNA interferes with binding between the RNA transcribed from the at least one regulatory element and the transcription factor in a way that does not directly interfere with binding of the transcription factor to its binding site in the at least one regulatory element (i.e., the peptide, polypeptide, or protein encoded by the mRNA binds to the transcription factor at a site that is distinct from, or otherwise does not interfere with, the DNA binding domain of the transcription factor).

In some embodiments, the synthetic, modified mRNA (or other synthetic nucleic acid) is capable of evading an innate immune response of a cell, tissue, or subject in which the mRNA is introduced and/or does not induce, or has decreased ability to induce, an innate immune response, e.g., as compared to a corresponding unmodified mRNA. Because the synthetic nucleic acids (e.g., mRNAs) are modified, e.g., to enhance the efficiency of their translation, their intracellular retention, stability, and also possess decreased immunogenicity, the synthetic, modified nucleic acids (e.g., mRNAs) having one or more these properties also may also be referred to in some embodiments as “enhanced nucleic acids.” In some embodiments, the peptide, polypeptide, or protein encoded by the synthetic, modified mRNA comprises one or more post-translational modifications (e.g., those present in mammalian, e.g., human cells).

The modified mRNAs can be engineered to encode a peptide, polypeptide, or protein (e.g., antibody or antibody fragment) that lacks a secretory signal sequence, such that the translated peptide, polypeptide, or protein is not secreted from the target cell in which it is produced. The modified mRNAs can be engineered to encode a peptide, polypeptide, or protein (e.g. antibody or antibody fragment) containing a nuclear localization signal sequence that allows for entrance of the peptide, polypeptide, or protein into the nucleus of a cell of interest (e.g., target cell) where transcription of the target gene regulated by a transcription factor of interest is located. In some embodiments, the nuclear localization signal sequence (NLS) comprises a canonical NLS. In some embodiments, the NLS comprises a single stretch of five to six basic amino acids (e.g., exemplified by the simian virus (SV) 40 large T antigen NLS). In some embodiments, the NLS comprises a bipartite NLS composed of two basic amino acids, a spacer region of 10-12 amino acids, and a cluster in which three of five amino acids must be basic (e.g., as exemplified by nucleoplasmin).

The modified mRNAs can be engineered to encode peptides, polypeptides, or proteins employing NLS-independent mechanisms for passage through the nuclear pore complex into the nucleus of target cells of interest. Examples of such NLS-independent mechanisms include passive diffusion of small proteins (<30-40 kDa), distinct nuclear-directing motifs [D. Christophe, C. Christophe-Hobertus, B. Pichon, Cell Signal 12, 337 (May, 2000), incorporated herein by reference], interaction with NLS-containing proteins, or alternatively, a direct interaction with the nuclear pore proteins (NUPs); [L. Xu, J. Massague, Nat Rev Mol Cell Biol 5, 209 (March, 2004), incorporated herein by reference]. In some embodiments, the mRNA encodes a peptide, polypeptide, or protein that contains nuclear translocation sequences from signaling proteins that translocate into the nucleus upon stimulation, in an NLS-independent manner, so that the peptide, polypeptide, or protein can translocate to the nucleus. Such translocation may occur via direct interaction with NUPs. Examples of such signaling proteins include ERKs, MEKs and SMADs. In some embodiments, the modified mRNAs are engineered to lack consensus sequences that interact with exportin proteins that mediate rapid export of shuttling proteins from the nucleus (e.g., a nuclear export signal (NES), such as the NES consensus sequence of LXXLXXLXL (SEQ ID NO: 53; identified as having sequence identifier number 36 in U.S. Publication No. 2014/0212438, which is incorporated herein by reference in its entirety)). The peptides, polypeptides, and proteins encoded by the modified mRNAs can be engineered to contain nuclear retention signals that enable the peptides, polypeptides, and proteins encoded by the modified mRNAs to remain in the nucleus once transported there.

In some embodiments, the mRNA encodes a peptide, polypeptide, or protein having nuclear targeting activity that comprises a nuclear targeting sequence less than or equal to 20 amino acids in length comprising X₁, X₂, X₃, wherein X₁ and X₃ are each independently selected from the group consisting of serine, threonine, aspartic acid and glutamic acid, and wherein X₂ is proline, as described in U.S. Publication No. 2014/0212438, which is incorporated herein by reference).

The peptides, polypeptides, and proteins encoded by the modified mRNAs can be engineered to be conjugated to a nuclear localization sequence-binding protein antibody or fragment thereof (i.e., so that when the peptide, polypeptide, or protein is translated in a target cell of interest, the anti-nuclear localization sequence-binding protein antibody portion of the peptide, polypeptide, or protein binds to a nuclear localization sequence and transports the peptide, polypeptide, or protein into the nucleus of the target cell of interest.

It should be appreciated that the modified mRNAs can be engineered to encode peptides, polypeptides, and proteins (e.g., antibodies or antibody fragments) which contain nuclear localization signal sequences, and/or nuclear retention signal sequences, and/or lack secretory signal sequences, and/or nuclear export signal sequences.

The synthetic, modified mRNAs of use herein may be prepared according to any available technique including, but not limited to chemical synthesis, enzymatic synthesis, which is generally termed in vitro transcription, enzymatic or chemical cleavage of a longer precursor, etc. Methods of synthesizing RNAs are known in the art (see, e.g., Gait, M. J. (ed.) Oligonucleotide synthesis: a practical approach, Oxford [Oxfordshire], Washington, D.C.: IRL Press, 1984; and Herdewijn, P. (ed.) Oligonucleotide synthesis: methods and applications, Methods in Molecular Biology, v. 288 (Clifton, N.J.) Totowa, N.J.: Humana Press, 2005; both of which are incorporated herein by reference).

“Synthetic, modified mRNA” and “modified mRNA” are used interchangeably herein. Modified mRNAs of use herein (e.g., encoding a peptide, polypeptide, or protein that interferes with binding between the transcribed RNA and a transcription factor of interest need not be uniformly modified along the entire length of the molecule. Different nucleotide modifications and/or backbone structures may exist at various positions in the mRNA. Other components of nucleic acid are optional, and may be beneficial in some embodiments. For example, a 5′ untranslated region (UTR) and/or a 3′UTR may be provided, wherein either or both may independently contain one or more different nucleoside modifications. In such embodiments, nucleoside modifications may also be present in the translatable region. Also contemplated are nucleic acids containing a Kozak sequence. In some embodiments, modified mRNA, e.g., in vitro transcribed mRNA, comprises a polyA tail at its 3′ end. Methods of adding a polyA tail to mRNA are known in the art, e.g., enzymatic addition via polyA polymerase or ligation with a suitable ligase.

One of ordinary skill in the art will appreciate that the nucleotide analogs or other modification(s) may be located at any position(s) of a mRNA such that the function of the nucleic acid is not substantially decreased. A modification may also be a 5′ or 3′terminal modification. The mRNA may contain at a minimum one and at maximum 100% modified nucleotides, or any intervening percentage, such as at least about 50% modified nucleotides, at least about 55% modified nucleotides, at least about 60% modified nucleotides, at least about 65% modified nucleotides, at least about 70% modified nucleotides, at least about 75% modified nucleotides, at least about 80% modified nucleotides, at least about 85% modified nucleotides, or at least about 90% modified nucleotides.

In some embodiments, the synthetic, modified mRNA encoding a peptide, polypeptide, or protein that interferes with binding between the RNA transcribed from at least one regulatory element and the transcription factor that binds to the RNA and the at least one regulatory element comprises at least one nucleoside selected from the group consisting of pyridin-4-one ribonucleoside, 5-aza-uridine, 2-thio-5-aza-midine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taulinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudomidine, dihydrouridine, dihydropseudouridine, 2-thio-dihydromidine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the synthetic, modified mRNA encoding a peptide, polypeptide, or protein that interferes with binding between the RNA transcribed from at least one regulatory element and the transcription factor that binds to the RNA and the at least one regulatory element comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the synthetic, modified mRNA encoding a peptide, polypeptide, or protein that interferes with binding between the RNA transcribed from at least one regulatory element and the transcription factor that binds to the RNA and the at least one regulatory element comprises at least one nucleoside selected from the group consisting of 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N-6-(cis-hydroxyisopentenyl) adenosine, N6-glycinylcarbamoyladenosine, N6-threonylcarbamoyladenosine, 2-methylthio-N-6-threonyl carbamoyladenosine, N6,N6-dimethyladenosine, 7-methyladenine, 2-methylthio-adenine, and 2-methoxy-adenine. In some embodiments, the synthetic, modified mRNA encoding a peptide, polypeptide, or protein that interferes with binding between the RNA transcribed from at least one regulatory element and the transcription factor that binds to the RNA and the at least one regulatory element comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

Generally, the length of a modified mRNA of the present disclosure is suitable for peptide, polypeptide, or protein production in a cell (e.g., a mammalian cell, e.g., human cell). For example, the modified mRNA is of a length sufficient to allow translation of at least a dipeptide in a cell. In one embodiment, the length of the modified mRNA is greater than 30 nucleotides. In another embodiment, the length is greater than 35 nucleotides. In another embodiment, the length is at least 40 nucleotides. In another embodiment, the length is at least 45 nucleotides. In another embodiment, the length is at least 55 nucleotides. In another embodiment, the length is at least 60 nucleotides. In another embodiment, the length is at least 60 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In some embodiments the length is no more than about 500 nucleotides, 750 nucleotides, 1000 nucleotides (1 kB), 2 kB, 3 kB, 4 kB, 5 kB, 6 kB, 7 kB, 8 kB, 9 kB, or 10 kB. In various embodiments the length can range from any lower limit to any upper limit that is greater than the lower limit.

In some embodiments, the modified mRNA encodes a peptide, polypeptide, or protein that binds to the transcription factor in a manner that prevents the transcription factor from binding to the RNA transcribed from the at least one regulatory element. In some embodiments, the peptide, polypeptide, or protein prevents the transcription factor from binding to the RNA transcribed from the at least one regulatory element, but does not prevent the transcription factor from directly binding to the at least one regulatory element (e.g., the peptide, polypeptide, or protein binds to the RNA binding domain or a site in proximity to the RNA binding domain of the transcription factor, but does not bind to the DNA binding domain or a site in proximity to the DNA binding domain of the transcription factor of interest). In some embodiments, the modified mRNA encodes a peptide, polypeptide, or protein that binds to the transcription factor at the same site that the RNA transcribed from at least one regulatory element would bind to the transcription factor. In some embodiments, modified mRNA encodes a peptide, polypeptide, or protein that binds to at least a portion of the same site that the RNA transcribed from at least one regulatory element would bind to the transcription factor (i.e., the agent binds to one or more amino acids of the transcription factor binding site for the RNA transcribed from the at least one regulatory element, but does not bind to all of the amino acids of such site). In some embodiments, the modified mRNA encodes a peptide, polypeptide, or protein that binds to the transcription factor in proximity to where RNA transcribed from at least one regulatory element binds to the transcription factor, but the agent masks the RNA binding site so the RNA can no longer bind to the transcription factor. In some embodiments, the modified mRNA encodes a peptide, polypeptide, or protein that binds to the transcription factor away from where the RNA transcribed from at least one regulatory element binds to the transcription factor, but the agent causes the transcription factor to change its conformation such that the RNA transcribed from at least one regulatory element can no longer bind to the transcription factor. In some embodiments, binding of the peptide, polypeptide, or protein (encoded by the mRNA) to the transcription factor affects another protein or cofactor that interacts with the transcription factor and the other protein or cofactor inhibits the RNA transcribed from at least one regulatory element from binding to the transcription factor.

In some embodiments, the modified mRNA encodes a peptide, polypeptide or protein of interest that binds to the transcription factor and has a length equal to the length of the RNA binding domain of a transcription factor of interest. In some embodiments, the transcription factor of interest in any aspect described herein is selected from the group consisting of YY1, KLF4, Thap11, REST, PRDM14, CTCF, STAT1, TLS/FUS, BRCA1, DLX2, ESR1, KIN, KU, NACA, NCL, NFKB1, NFYA, NR3C1, RARA, RUNX1, SOX2, TCF7, and p53. In some embodiments, the length of a modified mRNA encoding a peptide, polypeptide or protein of interest herein is equal to the length that is sufficient to bind to the RNA binding domain of a transcription factor of interest. In some embodiments, the modified mRNA encodes a peptide, polypeptide or protein of interest that has a length equal to a portion of the length of the RNA binding domain of the transcription factor of interest (e.g., the length of the peptide, polypeptide, or protein is long enough to bind to the RNA binding domain of the transcription factor in a manner that interferes with binding of the transcription factor to the RNA transcribed from at least one regulatory element, but does not bind to or block any other portion of the transcription factor).

In some embodiments, the modified mRNA encodes a peptide, polypeptide or protein of interest that binds to the transcription factor and has a length equal to the length of the binding site in the transcribed RNA for the transcription factor of interest. In some embodiments, the modified mRNA encodes a peptide, polypeptide or protein of interest that binds to the transcription factor and has a length equal to a portion of the length of the binding site in the transcribed RNA for the transcription factor of interest.

In some embodiments, the modified mRNA encodes an antibody or antibody fragment thereof that binds to the transcription factor in a manner that prevents the transcription factor from binding to the RNA transcribed from the at least one regulatory element. In some embodiments, the antibody or antibody fragment prevents the transcription factor from binding to the RNA transcribed from the at least one regulatory element, but does not prevent the transcription factor from directly binding to the at least one regulatory element (e.g., the antibody or antibody fragment binds to the RNA binding domain or a site in proximity to the RNA binding domain of the transcription factor, but does not bind to the DNA binding domain or a site in proximity to the DNA binding domain of the transcription factor of interest).

The modified mRNAs may encode full length antibodies or smaller antibodies (e.g., both heavy and light chains). For example, mRNAs may be translated in a cell, tissue, or subject for expression of the heavy and light chains of an immunoglobulin protein (e.g., IgA, IgD, IgE, IgG, and IgM) or antigen-binding fragments thereof (e.g., which bind to a target of interest, e.g., that bind to RNA transcribed from a regulatory element or that bind to a transcription factor of interest and inhibit binding of the TF to RNA transcribed from a regulatory element. The immunoglobulin proteins may be fully human, humanized, or chimeric immunoglobulin proteins. In some embodiments, the mRNA encodes an immunoglobulin protein or an antigen-binding fragment thereof, such as an immunoglobulin heavy chain, an immunoglobulin light chain, a single chain Fv, a fragment of an antibody, such as Fab, Fab′, or (Fab′)₂, or an antigen binding fragment of an immunoglobulin (See, e.g., US Publication No. 2013/0244282, which is incorporated herein by reference in its entirety). It should be appreciated that a single mRNA may be engineered to encode more than one subunit (e.g. in the case of a single-chain Fv antibody). In certain embodiments, separate mRNA molecules encoding the individual subunits may be administered in separate transfer vehicles. In some embodiments, the mRNA may encode full length antibodies (both heavy and light chains of the variable and constant regions) or fragments of antibodies (e.g. Fab, Fv, or a single chain Fv (scFv). In some embodiments the mRNA may encode a single domain antibody or antigen binding fragment thereof.

In some embodiments, the modified mRNA encodes an antibody or antibody fragment thereof that binds to all or a portion of the RNA binding domain of a transcription factor of interest. In some embodiments, the modified mRNA encodes an antibody or antibody fragment that binds to the RNA binding domain of the transcription factor in a manner that interferes with binding of the transcription factor to the RNA transcribed from at least one regulatory element, but does not bind to or block any other portion of the transcription factor (e.g., the DNA binding domain). In some embodiments, the modified mRNA encodes an antibody or an antibody fragment that binds to the transcription factor at a portion of the RNA binding domain that interacts with the binding site in the transcribed RNA for the transcription factor of interest.

In some embodiments, the modified mRNA encodes a peptide, polypeptide, or protein that binds to the RNA transcribed from the at least one regulatory element in a manner that prevents the transcription factor from binding to the RNA transcribed from the at least one regulatory element. In some embodiments, the modified mRNA encodes a peptide, polypeptide, or protein that binds to the RNA in the region that the RNA normally binds to the transcription factor. In some embodiments, the modified mRNA encodes a peptide, polypeptide, or protein that binds to the RNA at a different site from where the RNA binds to the transcription factor, e.g., such that the agent may mask the site on the RNA that binds to the transcription factor. In some embodiments, the modified mRNA encodes an antibody or antibody fragment that binds to the RNA transcribed from the at least one regulatory element in a manner that prevents the transcription factor from binding to the RNA transcribed from the at least one regulatory element.

In some embodiments, the antibody or antibody fragment encoded by the modified mRNA comprises a specific RNA-binding antibody or antibody fragment thereof. In some embodiments, the antibody comprises a specific RNA-binding antibody having a four-amino acid code (see, e.g., Sherman et al., “Specific RNA-binding antibodies with a four-amino-acid code,” J Mol Biol. 2014; 426(10):2145-57, which is incorporated herein by reference in its entirety). Sherman and colleagues describe methods that can be adapted in accordance with the guidance provided herein to construct and screen specific RNA-binding antibodies or antibody fragments which are capable of binding with specificity for and affinity to RNAs transcribed from regulatory elements occupied by transcription factors of interest wherein the RNA-binding antibodies or antibody fragments interfere with binding between the transcribed RNA and the transcription factor of interest, and decrease transcription of the target gene regulated by the regulatory elements occupied by the transcription factor of interest. For example, Sherman and colleagues describe design of an RNA-targeting Fab library with a minimal amino acid composition (e.g., the Fabs comprise complementarity-determining region (CDR) loops consisting of only the amino acids Tyr (Y), Ser (S), Gly (G) and Arg (R), construction of the Fab library (referred to as a “YSGR Min library” using a single Fab framework (P4-P6 binding Fab2) using Kunkel mutagenesis, the selection of antibodies in the YSGR Min library against particular RNA targets, the screening of individual phage clones by enzyme-linked immunosorbent assay, the expression and characterization of the Fabs, specificity assays, DNA constructs of the RNAs, in vitro transcription for the preparation of RNAs, preparation of the stop template for library construction, phage display for the selection for RNAs, phage ELISA for RNAs, native EMSA and PACE, filter binding assays, and competitive filter binding assays, all of which are incorporated herein by reference.

In some embodiments, the specific RNA-binding antibody comprises RNA-binding antibodies comprising complementarity-determining region (CDR) loops consisting of only the amino acids Tyr (Y), Ser (S), Gly (G) and Arg (R). In some embodiments, the specific RNA-binding antibody comprises RNA-binding antibodies comprising complementarity-determining region (CDR) loops consisting of only the amino acids Y, S, G and X, where X is any amino acid (see, e.g., Ye et al., “Synthetic antibodies for specific recognition and crystallization of structured RNA,” Proc Natl Acad Sci USA 2008; 105:82-7, which is incorporated herein by reference). In some embodiments, the specific RNA-binding antibody comprises RNA-binding antibodies comprising complementarity-determining region (CDR) loops consisting of only the amino acids Y, S, G, R, and X, wherein X is any amino acid (see, e.g., Koldobskaya, et al., “A portable RNA sequence whose recognition by a synthetic antibody facilitates structural determination,” Nat Struct Mol Biol 2011; 18:100-6, which is incorporated herein by reference in its entirety).

In some embodiments, phage display (or another display technology such as ribosome display, yeast display, bacterial display, mRNA display (e.g., using a cell-free system)) may be used to identify antibodies, peptides, or other proteins that bind to the RNA transcribed from a regulatory element or to a transcription factor that binds to RNA transcribed from at least one regulatory element. The presently disclosed subject matter contemplates modified nucleic acids (e.g., DNA, mRNA) encoding such antibodies, peptides, or proteins.

In some embodiments, the synthetic, modified mRNA encodes a variant peptide, polypeptide, or protein that has a certain identity with a reference peptide, polypeptide, or protein sequence. For example, the presently disclosed subject matter contemplates synthetic, modified mRNA encoding variants of a transcription factor of interest, i.e., a transcription factor that binds to RNA transcribed from at least one regulatory element and the at least one regulatory element. The term “identity” as known in the art, refers to a relationship between the sequences of two or more peptides, as determined by comparing the sequences. In the art, “identity” also means the degree of sequence relatedness between peptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Prut 1, Griffin, A. M., and Gtiffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carrillo et al., SIAM J. Applied Math. 48, 1073 (1988).

In some embodiments, the peptide, protein, or polypeptide variant has at least one activity that is the same or similar to an activity as the reference peptide, polypeptide, or protein (e.g., the peptide, protein, or polypeptide encoded by the synthetic, modified mRNA can bind to the same RNA transcribed from the at least one regulatory element as a transcription factor of interest). For example, the sequence of the mRNA encoding the peptide, protein, or polypeptide variant can be identical or similar to the RNA binding domain of a transcription factor of interest. In some embodiments, the peptide, protein, or polypeptide variant has at least one activity that is the same or similar to an activity as the reference peptide, polypeptide, or protein, but lacks at least one other activity of the reference peptide, polypeptide, or protein (e.g., the peptide, protein, or polypeptide encoded by the synthetic, modified mRNA can bind to the same RNA transcribed from the at least one regulatory element as a transcription factor of interest, but is not capable of binding to the at least one regulatory element). For example, the sequence of the mRNA encoding the peptide, protein, or polypeptide variant can be identical or similar to the RNA binding domain of a transcription factor of interest, but lack the DNA binding domain of the transcription factor of interest (e.g., the amino acids comprising the DNA binding domain can be deleted). In some embodiments, the sequence of the mRNA encoding the peptide, polypeptide, or protein variant can be identical or similar to the RNA binding domain of a transcription factor of interest, and the sequence of mRNA encoding the DNA binding domain of the transcription factor of interest can include one or more modifications (e.g., insertions, deletions, mutations) that prevent the DNA binding domain from binding to the at least one regulatory element. In some embodiments, the variant has an altered activity (e.g., increased or decreased) relative to a reference peptide, polypeptide, or protein (e.g., a transcription factor of interest). For example, an mRNA encoding a transcription factor of interest can be designed to exhibit increased affinity for binding to the transcribed RNA relative to the transcription factor of interest and/or decreased affinity for binding to the at least one regulatory element. Generally, variants of a particular peptide, polynucleotide, protein, or polypeptide of the disclosure will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular reference polynucleotide or polypeptide as determined by sequence alignment programs and parameters described herein and known to those skilled in the art.

The presently disclosed subject matter contemplates mRNAs encoding peptides, polypeptides, or proteins that comprise RNA binding domains that are homologous the RNA binding domain of a transcription factor of interest. In some embodiments, the modified RNA encodes a peptide, polypeptide, or protein that comprises a domain that is at least 50%, at least 55%, at least 60%, at least 65%, at least 70%, or at least 75%, least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% identical to the RNA binding domain of transcription factor selected from the group consisting of YY1, KLF4, Thap11, REST, PRDM14, CTCF, STAT1, TLS/FUS, BRCA1, DLX2, ESR1, KIN, KU, NACA, NCL, NFKB1, NFYA, NR3C1, RARA, RUNX1, SOX2, TCF7, and p53.

In some embodiments, the peptides, polypeptides, or proteins encoded by the modified mRNA comprise an RNA binding domain that is homologous to the RNA binding domain of a transcription factor of interest, but either lack the corresponding DNA binding domain or contain a DNA binding domain that has a DNA binding domain that has been altered to diminish its binding affinity for the at least one regulatory element (e.g., the DNA binding domain binds with a lesser affinity for the at least one regulatory element as compared to the DNA binding domain of the transcription factor of interest. In some embodiments, the modified RNA encodes a peptide, polypeptide, or protein that comprises a RNA binding domain that is at least 50%, at least 55%, at least 60%, at least 65%, at least 70%, or at least 75%, least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% identical to the RNA binding domain of transcription factor selected from the group consisting of YY1, KLF4, Thap11, REST, PRDM14, CTCF, STAT1, TLS/FUS, BRCA1, DLX2, ESR1, KIN, KU, NACA, NCL, NFKB1, NFYA, NR3C1, RARA, RUNX1, SOX2, TCF7, and p53, and lacks the corresponding DNA binding domain. In some embodiments, the modified RNA encodes a peptide, polypeptide, or protein that comprises a RNA binding domain that is at least 50%, at least 55%, at least 60%, at least 65%, at least 70%, or at least 75%, least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% identical to the RNA binding domain of transcription factor selected from the group consisting of YY1, KLF4, Thap11, REST, PRDM14, CTCF, STAT1, TLS/FUS, BRCA1, DLX2, ESR1, KIN, KU, NACA, NCL, NFKB1, NFYA, NR3C1, RARA, RUNX1, SOX2, TCF7, and p53, and contains a DNA binding domain that is no greater than 5%, no greater than 10%, no greater than 15%, no greater than 20%, no greater than 25%, no greater than 30%, no greater than 35%, no greater than 40%, or no greater than 45% identical to the DNA binding domain of the transcription factor.

The presently disclosed subject matter contemplates modified mRNAs encoding peptides, polypeptides, or proteins that are homologous to RNA binding domains of a transcription factor of interest. In some embodiments, the modified RNA encodes a peptide, polypeptide, or protein that is at least 50%, at least 55%, at least 60%, at least 65%, at least 70%, or at least 75%, least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% identical to the RNA binding domain of transcription factor selected from the group consisting of YY1, KLF4, Thap11, REST, PRDM14, CTCF, STAT1, TLS/FUS, BRCA1, DLX2, ESR1, KIN, KU, NACA, NCL, NFKB1, NFYA, NR3C1, RARA, RUNX1, SOX2, TCF7, and p53.

As recognized by those skilled in the art, protein fragments, functional protein domains, and homologous proteins are also considered to be within the scope of this disclosure. For example, provided herein is any protein fragment of a reference protein (meaning an mRNA encoding a polypeptide sequence at least one amino acid residue shorter than a reference polypeptide sequence but otherwise identical) about 10, 20, 30, 40, 50, 60, 70, 80, 90, 100, or greater than 100 amino acids in length. In another example, any protein that includes a stretch of about 20, about 30, about 40, about 50, or about 100 amino acids, which are about 40%, about 50%, about 60%, about 70%, about 80%, about 90%, about 95%, or about 100% identical to any of the sequences described herein, can be utilized in accordance with the disclosure. In certain embodiments, a protein sequence to be utilized in accordance with the disclosure includes 2, 3, 4, 5, 6, 7, 8, 9, 10, or more mutations as shown in any of the sequences referenced herein.

In some embodiments, the presently disclosed subject matter provides polynucleotide libraries containing nucleoside modifications, wherein the polynucleotides individually contain a first nucleic acid sequence encoding a peptide, polypeptide, or protein, such as an antibody, protein binding partner, scaffold protein, and other polypeptides (e.g., variants of a transcription factor of interest that can bind to RNA transcribed from regulatory elements of their naturally occurring counterparts (i.e., wild type transcription factors) but are unable to bind to the at least one regulatory element from which the RNA is transcribed and/or bind to the at least one regulatory element from which the RNA is transcribed with a lesser affinity compared to the wild type transcription factor). It should be appreciated that the library can comprise any of the modified mRNA described herein. Typically, the polynucleotides are modified mRNA in a form suitable for direct introduction into a target cell host, which in turn synthesizes the encoded peptide, polypeptide, or protein. In certain embodiments, multiple variants of a protein, each with different amino acid modification(s), are produced and tested to determine the best variant in terms of pharmacokinetics, stability, biocompatibility, and/or biological activity, or a biophysical property such as expression level. In some embodiments, the polynucleotides are assessed for their ability to be translated in the target cell host and to interfere with binding between a transcription factor of interest and RNA transcribed from at least one regulatory element occupied by the transcription factor of interest is assessed. Such a library may contain about 10, 10², 10³, 10⁴, 10⁵, 10⁶, 10⁷, 10⁸, 10⁹, or over 10⁹ possible variants (including substitutions, deletions of one or more residues, and insertion of one or more residues (e.g., variants of a transcription factor of interest comprising one or more sequence modifications to an RNA binding domain and/or DNA binding domain of the variant as compared to the transcription factor of interest, e.g., to alter the binding affinity (e.g., increase or decrease) of the RNA binding domain and/or DNA binding domain for its cognate RNA and/or DNA sequence relative to the binding affinity of the DNA binding domain and/or DNA binding domain of the transcription factor of interest.

In some embodiments, a modified mRNA of the presently disclosed subject matter encodes multiple peptides, polypeptides or proteins of interest that are capable of interfering with binding between the transcribed RNA and the transcription factor of interest. For example, the presently disclosed subject matter provides modified mRNAs containing an internal ribosome entry site (IRES). An IRES may act as the sole ribosome binding site, or may serve as one of multiplelibosome binding sites of an mRNA. An mRNA containing more than one functional ribosome binding site may encode several peptides or polypeptides that are translated independently by the ribosomes (“multicistronic mRNA”). When mRNAs are provided with an IRES, further optionally provided is at least a second translatable region. Examples of IRES sequences that can be used according to the disclosure include without limitation, those from picornaviruses (e.g. FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (STY) or cricket paralysis viruses (CrPV). In some embodiments a “self-cleaving” 2A peptide may be used instead of an IRES to, e.g., provide polycistronic expression from a single promoter. Self-cleaving 2A peptides were originally identified and characterized in apthovirus foot-and-mouth disease virus (FMDV). 2A oligopeptides are generally approximately 18-22 aa long and contain a highly conserved c-terminal D(V/I)EXNPGP (SEQ ID NO: 54) motif that mediates “ribosomal skipping” at the terminal 2A proline and subsequent amino acid (glycine). Examples of 2A peptide sequences that can be used according to the disclosure include without limitation, those from FMDV, equine rhinitis A virus (ERAV, porcine teschovirus-1 (PTV-1), and insect Thosea asigna virus (TaV).

In some embodiments, nucleic acids (e.g., enhanced nucleic acids) of interest herein (e.g., DNA constructs, synthetic RNAs, e.g., homologous or complementary RNAs described herein, mRNAs described herein, etc.) herein may be introduced into cells of interest via transfection, electroporation, cationic agents, polymers, or lipid-based delivery molecules well known to those of ordinary skill in the art.

In some embodiments, methods of the present disclosure enhance nucleic acid delivery into a cell population, in vivo, ex vivo, or in culture. For example, a cell culture containing a plurality of host cells (e.g., eukaryotic cells such as yeast or mammalian cells) is contacted with a composition that contains an enhanced nucleic acid having at least one nucleoside modification and, optionally, a translatable region. In some embodiments, the composition also generally contains a transfection reagent or other compound that increases the efficiency of enhanced nucleic acid uptake into the host cells. The enhanced nucleic acid exhibits enhanced retention in the cell population, relative to a corresponding unmodified nucleic acid. The retention of the enhanced nucleic acid is greater than the retention of the unmodified nucleic acid. In some embodiments, it is at least about 50%, 75%, 90%, 95%, 100%, 150%, 200%, or more than 200% greater than the retention of the unmodified nucleic acid. Such retention advantage may be achieved by one round of transfection with the enhanced nucleic acid, or may be obtained following repeated rounds of transfection.

The synthetic RNAs (e.g., modified mRNAs) of the presently disclosed subject matter may be optionally combined with a reporter gene (e.g., upstream or downstream of the coding region of the mRNA) which, for example, facilitates the determination of modified mRNA delivery to the target cells or tissues. Suitable reporter genes may include, for example, Green Fluorescent Protein mRNA (GFP mRNA), Renilla Luciferase mRNA (Luciferase mRNA), Firefly Luciferase mRNA, or any combinations thereof. For example, GFP mRNA may be fused with a mRNA encoding a nuclear localization sequence to facilitate confirmation of mRNA localization in the target cells where the RNA transcribed from the at least one regulatory element is taking place.

As used herein, the terms “transfect” or “transfection” mean the introduction of a nucleic acid, e.g., a synthetic RNA, e.g., modified mRNA into a cell, or preferably into a target cell. The introduced synthetic RNA (e.g., modified mRNA) may be stably or transiently maintained in the target cell. The term “transfection efficiency” refers to the relative amount of synthetic RNA (e.g., modified mRNA) taken up by the target cell which is subject to transfection. In practice, transfection efficiency may be estimated by the amount of a reporter nucleic acid product expressed by the target cells following transfection. Preferred embodiments include compositions with high transfection efficacies and in particular those compositions that minimize adverse effects which are mediated by transfection of non-target cells. In some embodiments, compositions of the present invention that demonstrate high transfection efficacies improve the likelihood that appropriate dosages of the synthetic RNA (e.g., modified mRNA) will be delivered to the target cell, while minimizing potential systemic adverse effects.

In some embodiments a cell may be genetically modified (in vitro or in vivo) (e.g., using a nucleic acid construct, e.g., a DNA construct) to cause it to express (i) an agent that modulates binding between nascent RNA transcribed from at least one regulatory element of a target gene and a transcription factor which binds to both the nascent RNA and the at least one regulatory element or (ii) an mRNA that encodes such an agent. For example, the present disclosure contemplates generating a cell or cell line that transiently or stably expresses an RNA that inhibits binding of the TF to nascent RNA transcribed from a regulatory element to which that TF binds or that transiently stably expresses an mRNA that encodes an antibody (or other protein capable of specific binding) that interferes with binding between a TF and nascent RNA transcribed from a regulatory element to which that TF binds. The genetically modified cells and constructs may be useful, e.g., in gene therapy approaches. For example, in some embodiments, such a nucleic acid construct is administered to an individual in need thereof. In other embodiments, cells (e.g., autologous) that have been contacted ex vivo with such a construct can be administered to an individual in need thereof. The construct may include a promoter operably linked to a sequence that encodes the agent or mRNA.

The synthetic RNA (e.g., modified mRNA) can be formulated with one or more acceptable reagents, which provide a vehicle for delivering such synthetic RNA (e.g., modified mRNA) to target cells. Appropriate reagents are generally selected with regard to a number of factors, which include, among other things, the biological or chemical properties of the synthetic RNA (e.g., modified mRNA), the intended route of administration, the anticipated biological environment to which such synthetic RNA (e.g., modified mRNA) will be exposed and the specific properties of the intended target cells. In some embodiments, transfer vehicles, such as liposomes, encapsulate the synthetic RNA (e.g., modified mRNA) without compromising biological activity. In some embodiments, the transfer vehicle demonstrates preferential and/or substantial binding to a target cell relative to non-target cells. In a preferred embodiment, the transfer vehicle delivers its contents to the target cell such that the synthetic RNA (e.g., modified mRNA) are delivered to the appropriate subcellular compartment, such as the cytoplasm.

In some embodiments, the transfer vehicle in the compositions of the invention is a liposomal transfer vehicle, e.g. a lipid nanoparticle. In one embodiment, the transfer vehicle may be selected and/or prepared to optimize delivery of the nucleic acid (e.g., synthetic RNA (e.g., modified mRNA)) to a target cell. For example, if the target cell is a hepatocyte the properties of the transfer vehicle (e.g., size, charge and/or pH) may be optimized to effectively deliver such transfer vehicle to the target cell, reduce immune clearance and/or promote retention in that target cell. Alternatively, if the target cell is the central nervous system (e.g., for the treatment of neurodegenerative diseases, the transfer vehicle may specifically target brain or spinal tissue), selection and preparation of the transfer vehicle must consider penetration of, and retention within the blood brain barrier and/or the use of alternate means of directly delivering such transfer vehicle to such target cell. In one embodiment, the compositions of the present invention may be combined with agents that facilitate the transfer of exogenous synthetic RNA (e.g., modified mRNA) (e.g., agents which disrupt or improve the permeability of the blood brain barrier and thereby enhance the transfer of exogenous mRNA to the target cells).

The use of liposomal transfer vehicles to facilitate the delivery of nucleic acids to target cells is contemplated by the present disclosure. Liposomes (e.g., liposomal lipid nanoparticles) are generally useful in a variety of applications in research, industry, and medicine, particularly for their use as transfer vehicles of diagnostic or therapeutic compounds in vivo (Lasic, Trends Biotechnol., 16: 307-321, 1998; Drummond et al., Pharmacol. Rev., 51: 691-743, 1999) and are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.).

In the context of the present disclosure, a liposomal transfer vehicle typically serves to transport the synthetic RNA (e.g., modified mRNA) to the target cell. For the purposes of the present invention, the liposomal transfer vehicles are prepared to contain the desired nucleic acids. The process of incorporation of a desired entity (e.g., a nucleic acid) into a liposome is often referred to as “loading” (Lasic, et al., FEBS Lett., 312: 255-258, 1992). The liposome-incorporated nucleic acids may be completely or partially located in the interior space of the liposome, within the bilayer membrane of the liposome, or associated with the exterior surface of the liposome membrane. The incorporation of a nucleic acid into liposomes is also referred to herein as “encapsulation” wherein the nucleic acid is entirely contained within the interior space of the liposome. The purpose of incorporating a synthetic RNA (e.g., modified mRNA) into a transfer vehicle, such as a liposome, is often to protect the nucleic acid from an environment which may contain enzymes or chemicals that degrade nucleic acids and/or systems or receptors that cause the rapid excretion of the nucleic acids. Accordingly, in a preferred embodiment of the present invention, the selected transfer vehicle is capable of enhancing the stability of the synthetic RNA (e.g., modified mRNA) contained therein. The liposome can allow the encapsulated synthetic RNA (e.g., modified mRNA) to reach the target cell and/or may preferentially allow the encapsulated synthetic RNA (e.g., modified mRNA) to reach the target cell, or alternatively limit the delivery of such synthetic RNA (e.g., modified mRNA) to other sites or cells where the presence of the administered synthetic RNA (e.g., modified mRNA) may be useless or undesirable. Furthermore, incorporating the synthetic RNA (e.g., modified mRNA) into a transfer vehicle, such as for example, a cationic liposome, also facilitates the delivery of such synthetic RNA (e.g., modified mRNA) into a target cell.

Liposomal transfer vehicles can be prepared to encapsulate one or more desired synthetic RNA (e.g., modified mRNA) such that the compositions demonstrate a high transfection efficiency and enhanced stability. While liposomes can facilitate introduction of nucleic acids into target cells, the addition of polycations (e.g., poly L-lysine and protamine), as a copolymer can facilitate, and in some instances markedly enhance the transfection efficiency of several types of cationic liposomes by 2-28 fold in a number of cell lines both in vitro and in vivo. (See N. J. Caplen, et al., Gene Ther. 1995; 2: 603; S. Li, et al., Gene Ther. 1997; 4, 891.) In some embodiments, the transfer vehicle is formulated as a lipid nanoparticle. As used herein, the phrase “lipid nanoparticle” refers to a transfer vehicle comprising one or more lipids (e.g., cationic lipids, non-cationic lipids, and PEG-modified lipids). Preferably, the lipid nanoparticles are formulated to deliver one or more synthetic RNAs (e.g., modified mRNAs) to one or more target cells.

Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine. In one embodiment, the transfer vehicle is selected based upon its ability to facilitate the transfection of a synthetic RNA (e.g., modified mRNA) to a target cell.

The present disclosure contemplates the use of lipid nanoparticles as transfer vehicles comprising a cationic lipid to encapsulate and/or enhance the delivery of synthetic RNA (e.g., modified mRNA) into the target cell, e.g., that will act as a depot for production of a peptide, polypeptide, or protein (e.g., antibody or antibody fragment) that interferes with binding between RNA transcribed from at least one regulatory element and a transcription factor that binds to the transcribed RNA and the at least one regulatory element. As used herein, the phrase “cationic lipid” refers to any of a number of lipid species that carry a net positive charge at a selected pH, such as physiological pH. The contemplated lipid nanoparticles may be prepared by including multi-component lipid mixtures of varying ratios employing one or more cationic lipids, non-cationic lipids and PEG-modified lipids. Several cationic lipids have been described in the literature, many of which are commercially available.

Suitable cationic lipids of use in the compositions and methods herein include those described in international patent publication WO 2010/053572, incorporated herein by reference, e.g., C12-200 described at paragraph [00225] of WO 2010/053572. In certain embodiments, the compositions and methods of the invention employ a lipid nanoparticles comprising an ionizable cationic lipid described in U.S. provisional patent application 61/617,468, filed Mar. 29, 2012 (incorporated herein by reference), such as, e.g., (15Z,18Z)-N,N-dimethyl-6-(9Z,12Z)-octadeca-9,12-dien-1-yl)tetracosa-15,18-dien-1-amine (HGT5000), (15Z,18Z)-N,N-dimethyl-6-((9Z,12Z)-octadeca-9,12-dien-1-yl)tetracosa-4,15,18-trien-1-amine (HGT5001), and (15Z,18Z)-N,N-dimethyl-6-((9Z,12Z)-octadeca-9,12-dien-1-yl)tetracosa-5,15,18-trien-1-amine (HGT5002).

In some embodiments, the cationic lipid N-[1-(2,3-dioleyloxy)propyl]-N,N,N-trimethylammonium chloride or “DOTMA” is used. (Felgner et al. (Proc. Nat'l Acad. Sci. 84, 7413 (1987); U.S. Pat. No. 4,897,355). DOTMA can be formulated alone or can be combined with the neutral lipid, dioleoylphosphatidyl-ethanolamine or “DOPE” or other cationic or non-cationic lipids into a liposomal transfer vehicle or a lipid nanoparticle, and such liposomes can be used to enhance the delivery of nucleic acids into target cells. Other suitable cationic lipids include, for example, 5-carboxyspermylglycinedioctadecylamide or “DOGS,” 2,3-dioleyloxy-N-[2(spermine-carboxamido)ethyl]-N,N-dimethyl-1-propanaminium or “DOSPA” (Behr et al. Proc. Nat.'l Acad. Sci. 86, 6982 (1989); U.S. Pat. No. 5,171,678; U.S. Pat. No. 5,334,761), 1,2-Dioleoyl-3-Dimethylammonium-Propane or “DODAP”, 1,2-Dioleoyl-3-Trimethylammonium-Propane or “DOTAP”. Contemplated cationic lipids also include 1,2-distearyloxy-N,N-dimethyl-3-aminopropane or “DSDMA”, 1,2-dioleyloxy-N,N-dimethyl-3-aminopropane or “DODMA”, 1,2-dilinoleyloxy-N,N-dimethyl-3-aminopropane or “DLinDMA”, 1,2-dilinolenyloxy-N,N-dimethyl-3-aminopropane or “DLenDMA”, N-dioleyl-N,N-dimethylammonium chloride or “DODAC”, N,N-distearyl-N,N-dimethylammonium bromide or “DDAB”, N-(1,2-dimyristyloxyprop-3-yl)-N,N-dimethyl-N-hydroxyethyl ammonium bromide or “DMRIE”, 3-dimethylamino-2-(cholest-5-en-3-beta-oxybutan-4-oxy)-1-(cis,cis-9,12-octadecadienoxy)propane or “CLinDMA”, 2-[5′-(cholest-5-en-3-beta-oxy)-3′-oxapentoxy)-3-dimethyl-1-(cis,cis-9′, 1-2′-octadecadienoxy)propane or “CpLinDMA”, N,N-dimethyl-3,4-dioleyloxybenzylamine or “DMOBA”, 1,2-N,N′-dioleylcarbamyl-3-dimethylaminopropane or “DOcarbDAP”, 2,3-Dilinoleoyloxy-N,N-dimethylpropylamine or “DLinDAP”, 1,2-N,N′-Dilinoleylcarbamyl-3-dimethylaminopropane or “DLincarbDAP”, 1,2-Dilinoleoylcarbamyl-3-dimethylaminopropane or “DLinCDAP”, 2,2-dilinoleyl-4-dimethylaminomethyl-[1,3]-dioxolane or “DLin-K-DMA”, 2,2-dilinoleyl-4-dimethylaminoethyl41,31-dioxolane or “DLin-K-XTC2-DMA”, and 2-(2,2-di((9Z,12Z)-octadeca-9,12-dien-1-yl)-1,3-dioxolan-4-yl)-N,N-dimethylethanamine (DLin-KC2-DMA)) (See, WO 2010/042877; Semple et al., Nature Biotech. 28:172-176 (2010)), or mixtures thereof (Heyes, J., et al., J Controlled Release 107: 276-287 (2005); Morrissey, D V., et al., Nat. Biotechnol. 23(8): 1003-1007 (2005); PCT Publication WO2005/121348A1).

The use of cholesterol-based cationic lipids is also contemplated by the present disclosure. Such cholesterol-based cationic lipids can be used, either alone or in combination with other cationic or non-cationic lipids. Suitable cholesterol-based cationic lipids include, for example, DC-Chol (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or ICE.

The skilled artisan will appreciate that various reagents are commercially available to enhance transfection efficacy. Suitable examples include LIPOFECTIN (DOTMA:DOPE) (Invitrogen, Carlsbad, Calif.), LIPOFECTAMINE (DOSPA:DOPE) (Invitrogen), LIPOFECTAMINE2000. (Invitrogen), FUGENE, TRANSFECTAM (DOGS), and EFFECTENE.

Also contemplated are cationic lipids such as the dialkylamino-based, imidazole-based, and guanidinium-based lipids. For example, certain embodiments are directed to a composition comprising one or more imidazole-based cationic lipids, for example, the imidazole cholesterol ester or “ICE” lipid (3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-(1H-imidazol-4-yl)propanoate, as represented by structure (I) below. In a preferred embodiment, a transfer vehicle for delivery of synthetic RNA (e.g., modified mRNA) may comprise one or more imidazole-based cationic lipids, for example, the imidazole cholesterol ester or “ICE” lipid (3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-(1H-imidazol-4-yl)propanoate, as represented by structure (I).

The imidazole-based cationic lipids are also characterized by their reduced toxicity relative to other cationic lipids. The imidazole-based cationic lipids (e.g., ICE) may be used as the sole cationic lipid in the lipid nanoparticle, or alternatively may be combined with traditional cationic lipids, non-cationic lipids, and PEG-modified lipids. The cationic lipid may comprise a molar ratio of about 1% to about 90%, about 2% to about 70%, about 5% to about 50%, about 10% to about 40% of the total lipid present in the transfer vehicle, or preferably about 20% to about 70% of the total lipid present in the transfer vehicle.

In some embodiments, the lipid nanoparticles comprise the HGT4003 cationic lipid 2-((2,3-Bis((9Z,12Z)-octadeca-9,12-dien-1-yloxy)propyl)disulfanyl)-N,N-dimethylethanamine, as represented by structure (II) below, and as further described in U.S. Provisional Application No. 61/494,745, filed Jun. 8, 2011, the entire teachings of which are incorporated herein by reference in their entirety.

In other embodiments the compositions and methods described herein are directed to lipid nanoparticles comprising one or more cleavable lipids, such as, for example, one or more cationic lipids or compounds that comprise a cleavable disulfide (S-S) functional group (e.g., HGT4001, HGT4002, HGT4003, HGT4004 and HGT4005), as further described in U.S. Provisional Application No. 61/494,745, the entire teachings of which are incorporated herein by reference in their entirety.

The use of polyethylene glycol (PEG)-modified phospholipids and derivatized lipids such as derivatized cerarmides (PEG-CER), including N-Octanoyl-Sphingosine-1-[Succinyl(Methoxy Polyethylene Glycol)-2000] (C8 PEG-2000 ceramide) is also contemplated by the present invention, either alone or preferably in combination with other lipids together which comprise the transfer vehicle (e.g., a lipid nanoparticle). Contemplated PEG-modified lipids include, but is not limited to, a polyethylene glycol chain of up to 5 kDa in length covalently attached to a lipid with alkyl chain(s) of C₆-C₂₀ length. The addition of such components may prevent complex aggregation and may also provide a means for increasing circulation lifetime and increasing the delivery of the lipid-nucleic acid composition to the target cell, (Klibanov et al. (1990) FEBS Letters, 268 (1): 235-237), or they may be selected to rapidly exchange out of the formulation in vivo (see U.S. Pat. No. 5,885,613). In some embodiments, exchangeable lipids comprise PEG-ceramides having shorter acyl chains (e.g., C14 or C18). The PEG-modified phospholipid and derivatized lipids of the present invention may comprise a molar ratio from about 0% to about 20%, about 0.5% to about 20%, about 1% to about 15%, about 4% to about 10%, or about 2% of the total lipid present in the liposomal transfer vehicle.

The present disclosure also contemplates the use of non-cationic lipids. As used herein, the phrase “non-cationic lipid” refers to any neutral, zwitterionic or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. Such non-cationic lipids may be used alone, but are preferably used in combination with other excipients, for example, cationic lipids. When used in combination with a cationic lipid, the non-cationic lipid may comprise a molar ratio of 5% to about 90%, or preferably about 10% to about 70% of the total lipid present in the transfer vehicle.

In some embodiments, the transfer vehicle (e.g., a lipid nanoparticle) is prepared by combining multiple lipid and/or polymer components. For example, a transfer vehicle may be prepared using C12-200, DOPE, chol, DMG-PEG2K at a molar ratio of 40:30:25:5, or DODAP, DOPE, cholesterol, DMG-PEG2K at a molar ratio of 18:56:20:6, or HGT5000, DOPE, chol, DMG-PEG2K at a molar ratio of 40:20:35:5, or HGT5001, DOPE, chol, DMG-PEG2K at a molar ratio of 40:20:35:5. The selection of cationic lipids, non-cationic lipids and/or PEG-modified lipids which comprise the lipid nanoparticle, as well as the relative molar ratio of such lipids to each other, is based upon the characteristics of the selected lipid(s), the nature of the intended target cells, the characteristics of the synthetic RNA (e.g., modified mRNA) to be delivered. Additional considerations include, for example, the saturation of the alkyl chain, as well as the size, charge, pH, pKa, fusogenicity and toxicity of the selected lipid(s). Thus the molar ratios may be adjusted accordingly. For example, in embodiments, the percentage of cationic lipid in the lipid nanoparticle may be greater than 10%, greater than 20%, greater than 30%, greater than 40%, greater than 50%, greater than 60%, or greater than 70%. The percentage of non-cationic lipid in the lipid nanoparticle may be greater than 5%, greater than 10%, greater than 20%, greater than 30%, or greater than 40%. The percentage of cholesterol in the lipid nanoparticle may be greater than 10%, greater than 20%, greater than 30%, or greater than 40%. The percentage of PEG-modified lipid in the lipid nanoparticle may be greater than 1%, greater than 2%, greater than 5%, greater than 10%, or greater than 20%.

In certain embodiments, the lipid nanoparticles of the present disclosure comprise at least one of the following cationic lipids: C12-200, DLin-KC2-DMA, DODAP, HGT4003, ICE, HGT5000, or HGT5001. In embodiments, the transfer vehicle comprises cholesterol and/or a PEG-modified lipid. In some embodiments, the transfer vehicles comprises DMG-PEG2K. In certain embodiments, the transfer vehicle comprises one of the following lipid formulations: C12-200, DOPE, chol, DMG-PEG2K; DODAP, DOPE, cholesterol, DMG-PEG2K; HGT5000, DOPE, chol, DMG-PEG2K, HGT5001, DOPE, chol, DMG-PEG2K.

The liposomal transfer vehicles for use in the compositions of the disclosure can be prepared by various techniques which are presently known in the art. Multi-lamellar vesicles (MLV) may be prepared conventional techniques, for example, by depositing a selected lipid on the inside wall of a suitable container or vessel by dissolving the lipid in an appropriate solvent, and then evaporating the solvent to leave a thin film on the inside of the vessel or by spray drying. An aqueous phase may then added to the vessel with a vortexing motion which results in the formation of MLVs. Uni-lamellar vesicles (ULV) can then be formed by homogenization, sonication or extrusion of the multi-lamellar vesicles. In addition, unilamellar vesicles can be formed by detergent removal techniques.

In certain embodiments, the compositions of the present disclosure comprise a transfer vehicle wherein the synthetic RNA (e.g., modified mRNA) is associated on both the surface of the transfer vehicle and encapsulated within the same transfer vehicle. For example, during preparation of the compositions of the present invention, cationic liposomal transfer vehicles may associate with the synthetic RNA (e.g., modified mRNA) through electrostatic interactions.

In certain embodiments, the compositions of the invention may be loaded with diagnostic radionuclide, fluorescent materials or other materials that are detectable in both in vitro and in vivo applications. For example, suitable diagnostic materials for use in the present invention may include Rhodamine-dioleoylphospha-tidylethanolamine (Rh-PE), Green Fluorescent Protein mRNA (GFP mRNA), Renilla Luciferase mRNA and Firefly Luciferase mRNA.

Selection of the appropriate size of a liposomal transfer vehicle must take into consideration the site of the target cell or tissue and to some extent the application for which the liposome is being made. In some embodiments, it may be desirable to limit transfection of the synthetic RNA (e.g., modified mRNA) to certain cells or tissues. For example, to target hepatocytes a liposomal transfer vehicle may be sized such that its dimensions are smaller than the fenestrations of the endothelial layer lining hepatic sinusoids in the liver; accordingly the liposomal transfer vehicle can readily penetrate such endothelial fenestrations to reach the target hepatocytes. Alternatively, a liposomal transfer vehicle may be sized such that the dimensions of the liposome are of a sufficient diameter to limit or expressly avoid distribution into certain cells or tissues. For example, a liposomal transfer vehicle may be sized such that its dimensions are larger than the fenestrations of the endothelial layer lining hepatic sinusoids to thereby limit distribution of the liposomal transfer vehicle to hepatocytes. Generally, the size of the transfer vehicle is within the range of about 25 to 250 nm, preferably less than about 250 nm, 175 nm, 150 nm, 125 nm, 100 nm, 75 nm, 50 nm, 25 nm or 10 nm.

A variety of alternative methods known in the art are available for sizing of a population of liposomal transfer vehicles. One such sizing method is described in U.S. Pat. No. 4,737,323, incorporated herein by reference. Sonicating a liposome suspension either by bath or probe sonication produces a progressive size reduction down to small ULV less than about 0.05 microns in diameter. Homogenization is another method that relies on shearing energy to fragment large liposomes into smaller ones. In a typical homogenization procedure, MLV are recirculated through a standard emulsion homogenizer until selected liposome sizes, typically between about 0.1 and 0.5 microns, are observed. The size of the liposomal vesicles may be determined by quasi-electric light scattering (QELS) as described in Bloomfield, Ann. Rev. Biophys. Bioeng., 10:421-450 (1981), incorporated herein by reference. Average liposome diameter may be reduced by sonication of formed liposomes. Intermittent sonication cycles may be alternated with QELS assessment to guide efficient liposome synthesis.

As used herein, the term “target cell” refers to a cell or tissue to which a composition of the invention is to be directed or targeted. For example, where it is desired to deliver a nucleic acid to a hepatocyte, the hepatocyte represents the target cell. In some embodiments, the compositions of the invention transfect the target cells on a discriminatory basis (i.e., do not transfect non-target cells). The compositions of the invention may also be prepared to preferentially target a variety of target cells, which include, but are not limited to, hepatocytes, epithelial cells, hematopoietic cells, epithelial cells, endothelial cells, lung cells, bone cells, stem cells, mesenchymal cells, neural cells (e.g., meninges, astrocytes, motor neurons, cells of the dorsal root ganglia and anterior horn motor neurons), photoreceptor cells (e.g., rods and cones), retinal pigmented epithelial cells, secretory cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes and tumor cells. In some embodiments, the target cells are deficient in a protein or enzyme of interest. In some embodiments the protein or enzyme of interest is encoded by a target gene, and the composition comprises an agent that increases expression of the target gene by stabilizing occupancy of a regulatory element of the target gene by a transcription factor.

The compositions of the invention may be prepared to preferentially distribute to target cells such as in the heart, lungs, kidneys, liver, and spleen. In some embodiments, the compositions of the invention distribute into the cells of the liver to facilitate the delivery and the subsequent expression of the synthetic RNA (e.g., modified mRNA) comprised therein by the cells of the liver (e.g., hepatocytes). The targeted hepatocytes may function as a biological “reservoir” or “depot” capable of producing a functional protein or enzyme (e.g., one that interferes with binding between a transcription factor of interest and a transcribed RNA). Accordingly, in one embodiment of the invention the liposomal transfer vehicle may target hepatocytes and/or preferentially distribute to the cells of the liver upon delivery. Following transfection of the target hepatocytes, the synthetic RNA (e.g., modified mRNA) loaded in the liposomal vehicle are translated and a functional protein product is produced. In other embodiments, cells other than hepatocytes (e.g., lung, spleen, heart, ocular, or cells of the central nervous system) can serve as a depot location for protein production.

The expressed or translated peptides, polypeptides, or proteins may also be characterized by the in vivo inclusion of native post-translational modifications which may often be absent in recombinantly-prepared proteins or enzymes, thereby further reducing the immunogenicity of the translated peptide, polypeptide, or protein.

The present disclosure also contemplates the discriminatory targeting of target cells and tissues by both passive and active targeting means. The phenomenon of passive targeting exploits the natural distributions patterns of a transfer vehicle in vivo without relying upon the use of additional excipients or means to enhance recognition of the transfer vehicle by target cells. For example, transfer vehicles which are subject to phagocytosis by the cells of the reticulo-endothelial system are likely to accumulate in the liver or spleen, and accordingly may provide means to passively direct the delivery of the compositions to such target cells.

The present disclosure contemplates active targeting, which involves the use of additional excipients, referred to herein as “targeting ligands” that may be bound (either covalently or non-covalently) to the transfer vehicle to encourage localization of such transfer vehicle at certain target cells or target tissues. For example, targeting may be mediated by the inclusion of one or more endogenous targeting ligands (e.g., apolipoprotein E) in or on the transfer vehicle to encourage distribution to the target cells or tissues. Recognition of the targeting ligand by the target tissues actively facilitates tissue distribution and cellular uptake of the transfer vehicle and/or its contents in the target cells and tissues (e.g., the inclusion of an apolipoprotein-E targeting ligand in or on the transfer vehicle encourages recognition and binding of the transfer vehicle to endogenous low density lipoprotein receptors expressed by hepatocytes). As provided herein, the composition can comprise a ligand capable of enhancing affinity of the composition to the target cell. Targeting ligands may be linked to the outer bilayer of the lipid particle during formulation or post-formulation. These methods are well known in the art. In addition, some lipid particle formulations may employ fusogenic polymers such as PEAA, hemagluttinin, other lipopeptides (see U.S. patent application Ser. Nos. 08/835,281, and 60/083,294, which are incorporated herein by reference) and other features useful for in vivo and/or intracellular delivery. In other some embodiments, the compositions of the present invention demonstrate improved transfection efficacies, and/or demonstrate enhanced selectivity towards target cells or tissues of interest. Contemplated therefore are compositions which comprise one or more ligands (e.g., peptides, aptamers, oligonucleotides, a vitamin or other molecules) that are capable of enhancing the affinity of the compositions and their nucleic acid contents for the target cells or tissues. Suitable ligands may optionally be bound or linked to the surface of the transfer vehicle. In some embodiments, the targeting ligand may span the surface of a transfer vehicle or be encapsulated within the transfer vehicle. Suitable ligands and are selected based upon their physical, chemical or biological properties (e.g., selective affinity and/or recognition of target cell surface markers or features.) Cell-specific target sites and their corresponding targeting ligand can vary widely. Suitable targeting ligands are selected such that the unique characteristics of a target cell are exploited, thus allowing the composition to discriminate between target and non-target cells. For example, compositions of the invention may include surface markers (e.g., apolipoprotein-B or apolipoprotein-E) that selectively enhance recognition of, or affinity to hepatocytes (e.g., by receptor-mediated recognition of and binding to such surface markers). Additionally, the use of galactose as a targeting ligand would be expected to direct the compositions of the present invention to parenchymal hepatocytes, or alternatively the use of mannose containing sugar residues as a targeting ligand would be expected to direct the compositions of the present invention to liver endothelial cells (e.g., mannose containing sugar residues that may bind preferentially to the asialoglycoprotein receptor present in hepatocytes). (See Hillery A M, et al. “Drug Delivery and Targeting: For Pharmacists and Pharmaceutical Scientists” (2002) Taylor & Francis, Inc.) The presentation of such targeting ligands that have been conjugated to moieties present in the transfer vehicle (e.g., a lipid nanoparticle) therefore facilitate recognition and uptake of the compositions of the present invention in target cells and tissues. Examples of suitable targeting ligands include one or more peptides, proteins, aptamers, small molecules, vitamins and oligonucleotides.

In some embodiments, the synthetic RNAs comprise at least one modification.

In some embodiments, at least 1%, at least 5%, at least 10%, at least 15%, at least 20%, at least 25%, at least 33%, at least 40%, at least 50%, at least 66%, at least 75%, at least 80%, at least 85%, at least 90%, or more of the nucleotides of the synthetic RNA comprise a modification. In some embodiments, the synthetic RNA comprises at least two, at least three, at least four, at least five, at least 10, at least 15, at least 20, at least 30, at least 35, at least 40, at least 45, at least 50, at least 55, at least 60, or more modifications, e.g., which can be the same modification throughout, or a combination of two, three, four, five, or more different modifications throughout.

In some embodiments, the composition comprises an agent which binds to the RNA in a manner that prevents the transcription factor from binding to the RNA. In some embodiments, the agent may bind to the RNA in the region that the RNA normally binds to the transcription factor. In some embodiments, the agent may bind to the RNA at a different site from where the RNA binds to the transcription factor, such that the agent may mask the site on the RNA that binds to the transcription factor or the agent may change the conformation of the RNA so that it no longer binds to the transcription factor.

In some embodiments, the agent is selected from the group consisting of small molecules, saccharides, peptides, proteins, peptidomimetics, nucleic acids, an extract made from biological materials selected from the group consisting of bacteria, plants, fungi, animal cells, and animal tissues, and any combination thereof.

In some embodiments, the agent is an RNA interfering agent selected from the group consisting of a ribozyme, guide RNA, small interfering RNA (siRNA), short hairpin RNA or small hairpin RNA (shRNA), microRNA (miRNA), post-transcriptional gene silencing RNA (ptgsRNA), short interfering oligonucleotide, antisense oligonucleotide, aptamer, and CRISPR RNA.

In some embodiments, the composition modifies at least one nucleotide of a DNA sequence in a manner that prevents RNA transcribed from the at least one regulatory element from binding to the transcription factor. For example, at least one nucleotide of a DNA sequence that is transcribed to produce RNA can be made such that the modification alters the sequence of the transcribed RNA, such that the transcribed RNA has a reduced affinity for the transcription factor. Of course, it should be appreciated that at least one nucleotide sequence of the DNA sequence encoding the transcription factor could be modified in a way that reduces the affinity of the transcription factor for the transcribed RNA but does not interfere with binding of the transcription factor to the at least one regulatory element. In some embodiments, the modification of at least one nucleotide may decrease the amount of RNA transcribed from the regulatory element such that the amount of RNA becomes limiting for the process of binding of the RNA to the transcription factor. In some embodiments, the modification of at least one nucleotide may essentially stop transcription of the RNA from the regulatory element so that RNA is no longer available for binding to the transcription factor.

In some embodiments, modification of at least one nucleotide may interfere with or not allow binding of at least one of the factors involved in transcription at the regulatory element, such that the amount of RNA transcribed from the regulatory element is reduced and/or the sequence of the RNA is altered such that the RNA binds less tightly to the transcription factor, resulting in a decrease in gene expression of the target gene. In some embodiments, modification of at least one nucleotide may increase binding of at least one of the factors involved in transcription at the regulatory element, such that the amount of RNA transcribed from the regulatory element is increased and/or the sequence of the RNA is altered such that the RNA binds more tightly to the transcription factor, resulting in an increase in gene expression of the target gene.

Non-limiting examples of compositions which modulate binding between the RNA and the transcription factor by modifying at least one nucleotide of a DNA sequence (e.g., a DNA sequence of the at least one regulatory element or DNA sequencing encoding RNA transcribed from the at least one regulatory element) include the CRISPR/Cas system, zinc finger nucleases (ZFNs), transcription activator-like effector nucleases (TALENS), and engineered meganuclease re-engineered homing endonucleases. In some embodiments, the composition comprises a CRISPR\Cas system, which relies upon the nuclease activity of the Cas9 protein (Makarova et al. (2011) Nat. Rev. Microbiol. 9:467-77) coupled with a synthetic guide RNA (gRNA) to make specific modifications in a genome (Barrangou et al. (2007) Science 315:1709-12; Brouns et al. (2008) Science 321:960-64; U.S. Pat. No. 8,771,945). In some embodiments, the composition comprises zinc finger nucleases (ZFNs), which comprise artificial restriction enzymes comprising a zinc finger protein (ZFP) and a nuclease cleavage domain ZFNs can be engineered to bind to a sequence of choice and therefore can be used to target sequences within a genome. (See, for example, Porteus, and Baltimore (2003) Science 300: 763; Miller et al. (2007) Nat. Biotechnol. 25:778-785; Sander et al. (2011) Nature Methods 8:67-69; Wood et al. (2011) Science 333:307); U.S. Patent Publication No. 20080159996). In some embodiments, the composition comprises Transcription Activator-Like Effector Nucleases (TALENs), which comprise TAL effector DNA-binding domains fused to a DNA cleavage domain (Wood et al. (2011) Science 333:307; Boch et al. (2009) Science 326:1509-1512; Moscou and Bogdanove (2009) Science 326:1501; Christian et al. (2010) Genetics 186:757-761; Miller et al. (2011) Nat. Biotechnol. 29:143-148; Zhang et al. (2011) Nat. Biotechnol. 29:149-153; Reyon et al. (2012) Nat. Biotechnol. 30:460-465; U.S. Patent Publication No. 20110145940), In some embodiments, the composition comprises engineered meganuclease re-engineered homing endonucleases.

The genome editing systems described hereinabove use artificially engineered nucleases to cut and create specific double-stranded breaks at a desired location(s) in the genome, which are then repaired by cellular endogenous processes such as, homologous recombination (HR), homology directed repair (HDR) and non-homologous end-joining (NHEJ). NHEJ directly joins the DNA ends in a double-stranded break, while HDR utilizes a homologous sequence as a template for regenerating the missing DNA sequence at the break point. In some embodiments, the regulatory element is modified via specialized nucleic acid replication processes associated with homology-directed repair (HDR). In such embodiments, at least one nucleotide of a DNA sequence to be modified is identified, and then a nucleic acid construct comprising a repair template with the desired modified nucleotide can be used with one of the above editing systems/compositions to modify the at least one nucleotide via homology-directed repair. In some embodiments, integration into the genome occurs through non-homology dependent targeted integration (e.g. “end-capture”). In some embodiments, at least one nucleotide is modified in accordance with the above genomic editing systems/compositions to increase the amount of RNA transcribed from the regulatory element or alter the sequence of the RNA such that it binds more tightly to the transcription factor, for example, to increase transcription of the target gene.

The presently disclosed subject matter also provides methods for screening the modifications of at least one nucleotide of a DNA sequence of at least one regulatory element which decrease binding of the transcription factor to the RNA transcribed from the modified regulatory element. In some embodiments, the presently disclosed subject matter provides methods of screening for a mutation, such as a single nucleotide polymorphism (SNP), in a DNA sequence encoding the at least one regulatory element or the RNA that is transcribed from the at least one regulatory element, whereby the resulting RNA binds to and stabilizes transcription factor occupancy on at least one allele of the at least one regulatory element. In some embodiments, the screening methods comprise identifying the transcription factor that binds both a regulatory element and the RNA transcribed from the regulatory element, and then determining whether the RNA transcribed from the regulatory element from one or both alleles stabilizes occupancy of the transcription factor at the regulatory element. If only one allele stabilizes occupancy of the transcription factor, steps can be performed to compare the two alleles (e.g., sequence alignment, genotyping) to determine whether there are any polymorphisms in one allele relative to another. Further, editing or fixing the polymorphism can be performed to see if that normalizes transcription from the edited allele.

In some embodiments, the presently disclosed subject matter provides methods to identify a disease for which RNA transcribed from a regulatory element increases transcription to cause or exacerbate the disease. In some embodiments, the methods comprise selecting a SNP at one or both alleles of a regulatory element for a target gene that is known to be associated with a disease, such as by searching a disease database (e.g., Online Mendelian Inheritance in Man (OMIM)) or by searching a database of genetic variation such as dbSNP or SNPedia), and then assaying to determine if the SNP increases transcription of the one or both alleles of the regulatory element.

In some embodiments, the presently disclosed subject matter provides methods to identify a disease for which RNA transcribed from a regulatory element decreases transcription to cause or exacerbate the disease. In some embodiments, the methods comprise selecting a SNP at one or both alleles of a regulatory element for a target gene that is known to be associated with a disease, such as by searching a disease database (e.g., Online Mendelian Inheritance in Man (OMIM)) or by searching a database of genetic variation such as dbSNP or SNPedia), and then assaying to determine if the SNP decreases transcription of the one or both alleles of the regulatory element.

In some embodiments, the presently disclosed subject matter provides methods for identifying modifications in a regulatory element that can be introduced to interfere with binding of the RNA transcribed from the regulatory element to the transcription factor. For example, in an embodiment, the DNA sequence is modified in cells using a genomic editing tool such as the CRISPR/Cas system and cross-linking immunoprecipitation (CLIP) and/or CLIP-sequencing is performed. A modification in the DNA sequence of the regulatory element that results in less PCR product as compared to a control in which modification of the DNA sequence did not occur is indicative that the modification decreased binding of the transcription factor to the RNA transcribed from the modified regulatory element.

In some embodiments, the modified regulatory element modulates transcription of a gene involved in a disease or disorder and the modification that decreases binding of the transcription factor to the RNA transcribed from the modified regulatory element can be used to prevent or treat the disease or disorder.

In some embodiments, the agent can bind to more than one component of the presently disclosed methods, such as at least two of RNA, the transcription factor, and at least one regulatory element. In some embodiments, the agent binds to the transcription factor, regulatory element, and/or the RNA via covalent bonding. In some embodiments, the agent binds to the transcription factor, regulatory element, and/or the RNA via non-covalent interactions, such as van der Waals interactions, electrostatic interactions (salt bridges), dipolar interactions (hydrogen bonding), and entropic effects (hydrophobic interactions).

The presently disclosed subject matter contemplates the use of compositions and/or agents that inhibit expression or activity of the exosome complex or a subunit or component thereof. Such agents are useful for therapeutic purposes, e.g., treatment of a disease, condition, or disorder which exhibit aberrantly high expression and/or disease-associated expression. The exosome or exosome complex is an intracellular protein complex that is capable of degrading various types of RNA molecules. In some embodiments, the composition comprises an agent which prevents exosomal degradation of untethered RNA in proximity to the at least one regulatory element or the transcriptional machinery. The term ‘untethered“, as in untethered RNA, refers to a molecule that is not fastened, bound, or connected to another molecule. In the context of nascent RNA transcribed from at least one regulatory element, untethered RNA refers to RNA that has been transcribed from the at least one regulatory element and is released from RNA polymerase (e.g., RNA Pol II). In some embodiments, methods using an agent which inhibits or prevents exosomal degradation of the untethered RNA result in an increase in untethered RNA and increased binding of the transcription factor to the untethered RNA, thereby titrating the transcription factor away from binding to nascent RNA. As used herein, the term “nascent RNA” refers to RNA that is still being transcribed or has just been transcribed by RNA polymerase. In some embodiments, the nascent RNA transcribed from the regulatory element is bound to RNA polymerase.

In some embodiments, the agent inhibits the expression and/or activity of the exosome or a subunit thereof. Examples of exosome components that can be inhibited include exosome component 1, exosome component 2, exosome component 3 (ExoKD), exosome component 4, exosome component 5, exosome component 6, exosome component 7, exosome component 8, exosome component 9, exosome component 10, and DIS3. In some embodiments, the agent inhibits a component of the exosome via RNA interference. In some embodiments, the agent comprises an shRNA against Exosc3.

In some embodiments, the presently disclosed subject matter provides synthetic RNA hybrid nucleic acids comprising DNA and RNA, e.g., oligonucleotides comprising one or more deoxyribonucleotides at either end or both and/or internally. In some embodiments, the presently disclosed subject matter provides oligonucleotides that promote RNase H-mediated degradation of the nascent RNA. RNase H degrades RNA in DNA/RNA hybrids. For example, antisense oligonucleotides comprising modifications at both ends (for biostability), e.g., 2′-O-methoxyethyl modifications at both ends, and a central gap of 10 unmodified nucleotides (deoxyribonucleotides) can be utilized to support RNase H activity (see, e.g., Wheeler et al., “Targeting nuclear RNA for in vivo correction of myotonic dystrophy,” Nature. 2012; 488(7409):111-115, which is incorporated herein by reference in its entirety). The deoxyribonucleic acids in the center of the oligonucleotide activate RNAse H and the end modifications stabilize the molecule. In some embodiments, one or more candidate oligonucleotides that are at least partly complementary to a nascent transcribed RNA of interest is tested to identify which of the candidate oligonucleotides effectively promote degradation of the nascent transcribed RNA.

In some embodiments, the presently disclosed subject matter provides a method of increasing transcription of a target gene by increasing the steady state levels of untethered RNA in proximity to the transcription factor, wherein the untethered RNA comprises an RNA which binds to the transcription factor at a site other than the DNA binding domain. In some embodiments, the untethered RNA binds to the transcription factor at a site that is in not in proximity to the DNA binding domain of the transcription factor.

In some embodiments, the presently disclosed subject matter provides methods for identifying agents that can outcompete the nascent RNA being transcribed. In some embodiments, the methods comprise assessing binding between RNA transcribed from at least one regulatory element and a transcription factor which binds to the RNA and to the at least one regulatory element in the presence or absence of a test agent, wherein decreased binding of the transcription factor to the RNA transcribed from the at least one regulatory element in the presence of the test agent as compared to the absence of the test agent indicates that the test agent is capable of outcompeting the nascent RNA being transcribed. Further competition experiments can be performed to determine whether the test agent is actually outcompeting the nascent RNA by binding to the transcription factor or whether the test agent is interfering with binding of the nascent RNA and the transcription factor without binding the transcription factor itself. Such an agent may further be used to destabilize expression of the target gene by being placed in proximity to the transcription factor to compete with the nascent RNA for binding to the transcription factor. In some embodiments, the agent is an RNA molecule. In some embodiments, this method is performed in vivo by growing cells (e.g., ESCs) with and without the agent and performing cross-linking immunoprecipitation (CLIP) and/or CLIP-sequencing. A decrease in PCR product in the presence of the agent as compared to the control without agent is indicative that the agent outcompeted the nascent RNA for binding to the transcription factor.

In some embodiments, the target gene comprises a gene for which increased or aberrant transcription is associated with a disease, condition, or disorder. In some embodiments, the disease, condition, or disorder is selected from the group consisting of cancer; genetic disorders; liver disorders, such as liver fibrosis and liver cancer; neurodegenerative disorders, such as Alzheimer's disease, amyotrophic lateral sclerosis (ALS), etc.; and autoimmune diseases, such as inflammatory bowel disease and rheumatoid arthritis. Cancer as used herein includes, but is not limited to, head cancer, neck cancer, head and neck cancer, lung cancer, breast cancer, prostate cancer, colorectal cancer, esophageal cancer, stomach cancer, leukemia/lymphoma, uterine cancer, skin cancer, endocrine cancer, urinary cancer, pancreatic cancer, gastrointestinal cancer, ovarian cancer, cervical cancer, and adenomas. In some embodiments, the cancer comprises a cancer for which an oncogene comprising a SNP is associated with increased expression (e.g., transcription) of the oncogene. In some embodiments, the cancer comprises a BRCA1-associated cancer. In some embodiments, the cancer comprises breast cancer comprising at least one SNP in at least one allele of the BRCA1 gene. In some embodiments, the cancer comprises ovarian cancer comprising at least one SNP in at least one allele of the BRCA1 gene.

Accordingly, in some embodiments, the presently disclosed subject matter also provides a method for treating a disease, condition, or disorder, the method comprising administering to a subject in need of treatment thereof, an agent that modulates binding between a ribonucleic acid (RNA) transcribed from at least one regulatory element of a target gene and a transcription factor which binds to both the RNA and the at least one regulatory element, wherein modulating binding between the RNA and the transcription factor modulates expression of the target gene. In some embodiments, the agent decreases binding between the RNA and the transcription factor to decrease expression of the target gene. In some embodiments, the agent increases binding between the RNA and the transcription factor to increase expression of the target gene. In some embodiments, the method includes identifying a subject having a disease, condition, or disorder exhibiting increased or aberrant transcription of a target gene driven by stabilization of transcription factor occupancy of at least one regulatory element due to binding of RNA transcribed from the at least one regulatory element to the transcription factor. In some embodiments, the method includes identifying a subject having a disease, condition, or disorder exhibiting decreased transcription of a target gene driven by destabilization of transcription factor occupancy of at least one regulatory element due to weakened or diminished binding of RNA transcribed from at least one regulatory element to the transcription factor. In some embodiments, the method includes identifying such diseases, conditions, or disorders. In some embodiments, the disease, condition, or disorder is selected from the group consisting of cancer, liver disorders, neurodegenerative disorders, metabolic disorders, and autoimmune diseases. As used herein, the term “treating” can include reversing, alleviating, inhibiting the progression of, preventing or reducing the likelihood of the disease, disorder, or condition to which such term applies, or one or more symptoms or manifestations of such disease, disorder or condition.

In some embodiments aberrantly increased expression of the target gene or aberrantly increased activity of a gene product of the target gene causes or contributes to the disease, and the method comprises inhibiting expression of the target gene by interfering with binding of the TF to RNA transcribed from a regulatory element of the target gene, e.g., by administering an agent that decreases such binding to a subject in need of treatment for the disease. In some embodiments aberrantly reduced expression of the target gene or aberrantly reduced activity of a gene product of the target gene causes or contributes to the disease, and the method comprises increasing expression of the target gene by increasing binding of the TF to RNA transcribed from a regulatory element of the target gene, e.g., by administering an agent that increases such binding to a subject in need of treatment for the disease.

In some embodiments, the target gene comprises an oncogene. Non-limiting examples of oncogenes include abl, Af4/hrx, akt-2, alk, alk/npm, aml1, aml1/mtg8, axl, bcl-2, bcl-3, bcl-6, bcr/abl, c-myc, dbl, dek/can, E2A/pbx1, egfr, enl/hrx, erg/TLS, erbB, erbB-2, ets-1, ews/fli-1, fms, fos, fps, gli, gsp, HER2/neu, hox11, hst, IL-3, int-2, jun, kit, KS3, K-sam, Lbc, lck, lmo1, lmo2, L-myc, lyl-1, lyt-10, lyt-10/C alpha1, mas, mdm-2, mll, mos, mtg8/aml1, myb, MYH11/CBFB, neu, N-myc, ost, pax-5, pbx1/E2A, pim-1, PRAD-1, raf, RAR/PML, rasH, rasK, rasN, rel/nrg, ret, rhom1, rhom2, ros, ski, sis, set/can, src, tal1, tal2, tan-1, Tiam1, TSC2, and trk.

In some embodiments the target gene encodes a protein. In some embodiments the protein is a transcription factor, a transcriptional co-activator or co-repressor, an enzyme (e.g., a kinase, phosphatase, acetylase, deacetylase, methylase, demethylase, protease), a chaperone, a co-chaperone, a heat shock protein, a receptor, a secreted protein, a transmembrane protein, a peripheral membrane protein, a soluble protein, a nuclear protein, a mitochondrial protein, a lysosomal protein, a growth factor, a cytokine (e.g., an interferon, an interleukin, a chemokine, a tumor necrosis factor), a hormone, an extracellular matrix protein, a motor protein, a cell adhesion molecule, a major or minor histocompatibility (MHC) protein, a transporter, a channel, an immunoglobulin (Ig) superfamily (IgSF) member, an integrin, a cadherin superfamily member, a selectin, a clotting factor, a complement factor, a pluripotency protein, or a tumor suppressor protein. In some embodiments the target gene encodes a protein that is a component of a multiprotein complex such as the ribosome, spliceosome, proteasome, or RNA-induced silencing complex. In some embodiments the target gene encodes a microRNA precursor or an RNA that is a component of a ribonucleoprotein complex.

In some embodiments, the target gene comprises at least one mutation in the at least one regulatory element, wherein the at least one mutation results in the transcription factor binding to RNA transcribed from the at least one regulatory element in a manner that stabilizes occupancy of the transcription factor at the at least one regulatory element, thereby increasing expression of the target gene. In some embodiments, the target gene comprises at least one mutation in the at least one regulatory element, wherein the at least one mutation results in diminished or weakened binding by the transcription factor to RNA transcribed from the at least one regulatory element, thereby decreasing expression of the target gene. In some embodiments, the at least one mutation comprises a single nucleotide polymorphism (SNP). Examples of SNPs can be found in the NCBI database of single nucleotide polymorphisms (dbSNP), SNPedia, and the like. Non-limiting examples of diseases associated with SNPs that are linked to regulatory elements include cancer, such as colorectal and gastric cancer (e.g., BRCA1 associated cancers); diabetes, such as type 2 diabetes; cardiovascular associated disease, such as coronary artery disease; neurodegenerative disorders, such as Parkinson's disease; and autoimmune disorders, such as inflammatory bowel disease.

In some embodiments, the presently disclosed subject matter provides a method for destabilizing the occupancy of the transcription factor at the at least one regulatory element wherein the regulatory element comprises at least one mutation that increases expression of the target gene, the method comprising using an agent that targets the mutated RNA that results from transcription of the regulatory element comprising at least one mutation. In this case, the agent can inhibit the mutated RNA, thereby inhibiting or blocking gene expression by destabilizing the occupancy of the transcription factor. As described hereinabove, a disease or disorder may be caused by increased transcription caused by at least one mutation at a regulatory element. Therefore, in some embodiments, an agent may be used to treat a disease caused by at least one mutation at a regulatory element.

In some embodiments, the presently disclosed subject matter provides a method of identifying a candidate agent that interferes with binding between RNA transcribed from at least one regulatory element and a transcription factor which binds to the RNA and to the at least one regulatory element, the method comprising assessing binding between RNA transcribed from at least one regulatory element and a transcription factor which binds to the RNA and to the at least one regulatory element in the presence and absence of a test agent, wherein decreased binding of the transcription factor to the RNA transcribed from the at least one regulatory element in the presence of the test agent as compared to the absence of the test agent indicates that the test agent is a candidate agent that interferes with binding between the RNA and the transcription factor. In some embodiments, the presently disclosed subject matter provides a method of identifying a candidate agent that promotes binding between RNA transcribed from at least one regulatory element and a transcription factor which binds to the RNA and to the at least one regulatory element, the method comprising assessing binding between RNA transcribed from at least one regulatory element and a transcription factor which binds to the RNA and to the at least one regulatory element in the presence and absence of a test agent, wherein increased binding of the transcription factor to the RNA transcribed from the at least one regulatory element in the presence of the test agent as compared to the absence of the test agent indicates that the test agent is a candidate agent that promotes binding between the RNA and the transcription factor. In some embodiments, binding is performed in a cell. In some embodiments, the method comprises performing cross-linking immunoprecipitation (CLIP) with the RNA and the transcription factor. In some embodiments, binding in the cell is assessed using RIP-eq. In some embodiments, binding in the cell is assessed using RIP-Chip.

Those skilled in the art will appreciate that a variety of cell-free binding assays can be used to identify a candidate agent. In some embodiments the method is performed in a cell-free composition comprising a TF that binds to a regulatory element from which RNA is transcribed, RNA whose sequence comprises at least a portion of the sequence of RNA transcribed from the regulatory element, and a candidate agent. The RNA may be incubated with the TF in the absence or presence of the candidate agent. Then, the TF or RNA is isolated from the composition (e.g., using immunoprecipitation). The amount of RNA bound to the TF in the presence of the candidate agent as compared with the amount of RNA bound to the TF in the absence of the candidate agent is determined. In some embodiments the RNA comprises or is conjugated to a detectable label (e.g., a fluorophore, radioactive atom, etc.), and RNA bound to the TF may be detected by detecting the detectable label. In some embodiments the RNA may be synthetically produced using chemical synthesis or an in vitro transcription system. In some embodiments the method comprises performing a high throughput screen to identify an agent that modulates binding between RNA transcribed from at least one regulatory element and a transcription factor which binds to the RNA and to the at least one regulatory element. In some embodiments the test agent is a small molecule, nucleic acid, peptide, etc.

In some embodiments, the methods further comprise identifying a transcription factor that binds to RNA transcribed from at least one regulatory element and to the at least one regulatory element. For example, the transcription factor can be identified by isolating the transcription factor-RNA complex formed from binding between RNA transcribed from at least one regulatory element and the transcription factor which binds to the RNA and to the at least one regulatory element and using a protein identification method such as mass spectrometry or protein sequencing to identify the transcription factor. In some embodiments, the methods further comprise identifying an RNA binding domain of the transcription factor. For example, once the transcription factor has been identified, its amino acid sequence can be compared to known sequences in databases to identify RNA recognition motifs, etc. In some embodiments, the methods further comprise identifying a consensus motif in the RNA transcribed from the at least one regulatory sequence for the RNA binding domain of the transcription factor.

In some embodiments, assessing binding comprises contacting a complex or mixture comprising the transcription factor, the at least one regulatory element, and the RNA transcribed from the at least one regulatory element with the test agent. In some embodiments, the methods further comprise assessing whether the test agent is capable of binding to the transcription factor at a site other than a DNA binding domain of the transcription factor.

In some embodiments, the test agent is selected from the group consisting of small molecules, saccharides, peptides, proteins, peptidomimetics, nucleic acids, an extract made from biological materials selected from the group consisting of bacteria, plants, fungi, animal cells, and animal tissues, and any combination thereof.

In some embodiments, the test agent comprises a decoy RNA as described herein.

In some embodiments, binding is performed in a cell. In some embodiments, the method comprises performing cross-linking immunoprecipitation (CLIP) with the RNA and the transcription factor. In some embodiments, the method comprises performing an EMSA assay. In some embodiments, the method comprises performing an immunoprecipitation assay.

In some aspects, the presently disclosed subject matter contemplates diagnostic and/or prognostic applications, for example, methods of diagnosing diseases, conditions, or disorders associated with aberrant transcription (e.g., increased or decreased) by detecting at least one modification in a DNA sequence encoding at least one regulatory element or the RNA transcribed from the at least one regulatory element, e.g., wherein the alteration of the DNA results in aberrant transcription (e.g., increased transcription, e.g., by stabilizing occupancy of a transcription factor which binds both the RNA and the at least one regulatory element, or decreased transcription, e.g., by destabilizing occupancy of a transcription factor which binds to both the RNA and the at least one regulatory element).

### II. Pharmaceutical Compositions and Administration

In another aspect, the present disclosure provides a pharmaceutical composition including an agent which interferes with binding between the RNA and the transcription factor alone or in combination with one or more additional therapeutic agents in admixture with a pharmaceutically acceptable excipient. One of skill in the art will recognize that the pharmaceutical compositions include the pharmaceutically acceptable salts of the compounds described above.

In therapeutic and/or diagnostic applications, the agent which interferes with binding between the RNA and the transcription factor for use within the methods of the presently disclosed subject matter can be formulated for a variety of modes of administration, including oral, systemic, and topical or localized administration. Techniques and formulations generally may be found in Remington: The Science and Practice of Pharmacy (20^{th} ed.) Lippincott, Williams & Wilkins (2000). The agents may be delivered, for example, in a timed- or sustained-low release form as is known to those skilled in the art. Techniques for formulation and administration may be found in Remington: The Science and Practice of Pharmacy (20^{th} ed.) Lippincott, Williams & Wilkins (2000).

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate. Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

An agent which interferes with binding between the RNA and the transcription factor may be formulated into liquid or solid dosage forms and administered systemically or locally. Suitable routes may include rectal, intestinal, or intraperitoneal delivery. Other suitable routes may include various forms of parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

The compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, and the preference and experience of the attending physician.

Pharmaceutically acceptable salts are generally well known to those of ordinary skill in the art, and may include, by way of example but not limitation, acetate, benzenesulfonate, besylate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). Pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate. Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Additional therapeutic agents may be administered together with the agent which interferes with binding between the RNA and the transcription factor within the methods of the presently disclosed subject matter. These additional agents may be administered separately, as part of a multiple dosage regimen, from the inhibitor-containing composition. Alternatively, these agents may be part of a single dosage form, mixed together with the inhibitor in a single composition.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the composition of the encapsulating matrix, the target tissue, and the like.

### III. Kits

The presently disclosed subject matter also relates to kits for practicing the methods of the presently disclosed subject matter. In general, a presently disclosed kit contains some or all of the components, reagents, supplies, and the like to practice a method according to the presently disclosed subject matter. In some embodiments, the term “kit” refers to any intended article of manufacture (e.g., a package or a container) comprising a composition or agent that modulates binding between RNA transcribed from at least one regulatory element and a transcription factor that binds to both the RNA and the at least one regulatory element, and a set of particular instructions for practicing the methods of the presently disclosed subject matter. The kit can be packaged in a divided or undivided container, such as a carton, bottle, ampule, tube, etc. The presently disclosed compositions can be packaged in dried, lyophilized, or liquid form. Additional components provided can include vehicles for reconstitution of dried components.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Methods

Murine Embryonic Stem Cells:

Bio-YY1 murine embryonic stem cells (mESCs) (Vella et al., 2012), control mESCs expressing only biotin ligase BirA (Vella et al., 2012), and bio-OCT4 ESCs (Kim et al., 2008) were grown on irradiated murine embryonic fibroblasts (MEFs) unless otherwise stated. Cells were grown under standard mESC conditions as described previously (Boyer et al., 2006). Briefly, cells were grown on 0.2% gelatinized (Sigma, G1890) tissue culture plates in ESC media; DMEMKO (Invitrogen, 10829-018) supplemented with 15% fetal bovine serum (Sigma, F4135-500), 1000 U/mL LIF (ESGRO, ESG1106), 100 μM nonessential amino acids (Invitrogen, 11140-050), 2 mM L-glutamine (Invitrogen, 25030-081), 100 U/mL penicillin, 100 μg/mL streptomycin (Invitrogen, 15140-122), and 8 nL/mL of 2-mercaptoethanol (Sigma, M7522).

Antibodies:

Anti-RNA polymerase II (phospho CTD Ser-2) (Millipore, 04-1571), anti-YY1 (Santa Cruz Biotechnology, SC-1703), anti-β-Tubulin (Millipore, 05-661), anti-Histone H3 (Abcam, ab1791), anti-OCT4 (Santa Cruz, 8628X), and anti-EXOSC3 (Abcam, ab156683) antibodies were used for Western blot analyses.

Actinomycin D, THZ1, Triptolide, and DRB Treatment:

Bio-YY1 mESCs were treated with 104 of Actinomycin D, 10 μM THZ1, or 104 triptolide prepared in 100% DMSO or with equal amount of DMSO by adding the drug or DMSO directly to the culture media and incubating cells with the drug for 6 hrs at 37° C. Cells were washed with PBS and used for GRO-seq or ChIP-seq analysis. In the DRB experiment, cells were treated with 100 μM DRB for 3 hrs and YY1 ChIP-qPCR was performed after the first 10, 20, and 30 minutes of incubation with the drug, whereas ChIP-seq was performed after 30-min of incubation with the drug. At the end of the 3 hr treatment, cells were washed two times with PBS, and YY1 ChIP-qPCR was performed after the first 10, 20, and 30 minutes after fresh media without the drug was added to the cells, whereas ChIP-seq was performed 30 minutes after fresh media was added as previously described (Schmieder and Edwards, 2011).

Affinity Purification of YY1-Associated DNA (YY1 ChIP-Seq):

Purification of YY1-associated chromatin was performed using bio-YY1 mESCs (Vella et al., 2012). Detailed protocol was described previously (Kim et al., 2009). Bio-YY1 is expressed at 10-20% of the endogenous YY1 protein level as has been estimated using quantitative Western blot analysis. mESCs were depleted of MEFs by splitting mESCs twice (1:5) when cells reached confluence onto newly gelatinized plates without MEFs and growing them until achieving confluence again. Approximately 5×107 mESCs was chemically crosslinked by the addition of one-tenth volume of fresh 11% formaldehyde solution for 10 minutes at room temperature. Cells were rinsed twice with 1×PBS and harvested using a silicon scraper and flash frozen in liquid nitrogen. Cells were stored at −80° C. prior to use. Cells were sonicated (Misonix Sonicator 3000) in sonication buffer (20 mM Tris-HCl pH 8.0, 150 mM NaCl, 2 mM EDTA, 1% Triton X-100, 0.1% SDS) for 10 cycles at 30 seconds each on ice at 21 watts (60 second pause between pulses). The resulting whole cell extract was cleared by centrifugation for 10 min at 12,000 g and then incubated overnight at 4° C. with 50 μl streptavidin sepharose high performance (GE Healthcare, 17-5113-01). Beads were washed 2× with 2% (vol/vol) SDS, 1× with 50 mM HEPES pH 7.5, 500 mM NaCl, 1 mM EDTA, 1% Triton X-100, 1× with 10 mM Tris-HCl pH 8.0, 250 mM LiCl, 1 mM EDTA, 0.5% NP40, 0.5% sodium deoxycholate, and 2× with TE buffer. The affinity-purified DNA was eluted and formaldehyde crosslinks were reversed overnight at 65° C. The DNA were purified by phenol/chloroform extraction and precipitated with ethanol. The ChIP-seq libraries were prepared with Illumina TruSeq DNA Sample Preparation kit, and sequenced on Illumina HiSeq 2000.

Analysis of the Affinity Purified YY1-Associated DNA (YY1 ChIP-Seq):

Acquired images were processed through the bundled Illumina image extraction pipeline, which identified polony positions, performed base calling and generated QC statistics. Sequences were aligned using Bowtie 1.0.1 (Langmead et al., 2009) to NCBI Build 37 (UCSC 37, mm9) of the mouse genome. Alignments were performed with the following parameters: -k 1 -m 1 -n 2 -p 4 -140 -best -sam. WIG files for visualization were made using MACS (Zhang et al., 2008) with parameters -w -S -space=50 -nomodel -shiftsize=200 -keep-dup=1. WIG files were uploaded and visualized on the UCSC Genome Browser (Kent et al., 2002). Peaks were identified using MACS with corresponding control and parameters -p 1e-9 and -keep-dup=1.

Genome-Wide YY1 Binding Motif Identification:

To identify DNA sequences in the mouse mm9 genome version predicted to bind YY1, FIMO was used (Grant et al., 2011). The genome was scanned with PWMs MA0142.1 from Jaspar (Bryne et al., 2008) for OCTSOX, and YY1 full from (Jolma et al., 2013) for YY1.

Targeting of ESCs with shRNA Against an Exosome Component:

The pLKO.1 shRNA constructs against firefly luciferase (as a negative control) and Exosc3 were a gift from Dr. Phillip Sharp. The targeted sequence in the Exosc3 mRNA is 5′-GGUGAAUUUCUUCCUGGCAGAUC-3′ (SEQ ID NO: 5). The targeted sequence in the firefly luciferase mRNA is 5′-GGACAUCACUUACGCUGAGU-3′ (SEQ ID NO: 6). The puromycin resistance gene in pLKO.1 construct was replaced with the hygromycin B resistance gene using BamHI and KpnI sites. About 3 μg pLKO.1 vector, 2.5 μg pMD2, and 7.5 μg pPAX plasmids were transfected into 6 million HEK293FT cells in T75 tissue culture flask using Lipofectamine 2000 (Life technologies) according to the manufacturer's instructions. Growth media was replaced with 10 ml fresh media 16 hrs post-transfection. Lentivirus-containing supernatants were harvested 48 hrs post-transfection and centrifuged at 1200 rpm for 20 min at room temperature to pellet cell debris. About 3 million mESCs in 4 ml complete growth media were incubated with 3 ml lentivirus-containing supernatant with polybrene (final concentration 6 μg/ml). The next day, the media was replaced with complete mESC culture media containing 500 μg/ml hygromycin B (Life Technologies, 10687-010). After 7 days of selection, mESCs were maintained in complete growth media containing 200 μg/ml hygromycin B.

Total RNA was isolated from 10 million mESCs (luciferase control or Exosc3 knockdown cells) using mirVana miRNA Isolation Kit (Ambion, AM1560) according to the manufacturer's protocol. About 7.6 μl of 1:100 fold dilution of ERCC RNA Spike-In Mix (Ambion, 4456740) was added to 1 μg total RNA. TruSeq Standed RiboZero libraries were then prepared and sequenced on Illumina HiSeq2000.

For YY1 ChIP-Seq experiments, mESCs targeted with control and Exosc3 shRNAs were counted and equal number of cells was plated 12 hrs before crosslinking.

RNA-Seq Analysis of Changes in Steady-State RNA Levels in ESCs Targeted with shRNA Against an Exosome Component:

RNA-Seq reads were aligned to the non-random mm9 version of the mouse reference genome with added ERCC spike-in chromosomes using tophat (Trapnell et al., 2009) with a GTF of mouse RefSeq genes provided as a parameter. FPKM values were calculated twice using RPKM_count from the RSeQC package (Wang et al., 2012). The initial run used all mouse RefSeq genes and ERCC spike-ins. This allowed determination that exosome knockdown did not cause a loss of mRNA expression, because the ERCC spike-ins added in ratio to cell number provide a normalization method capable of detecting global effects on expression. The expression of all RefSeq genes and ERCC probes was floored at 0.01 and a pseudocount of 0.1 was added to all entries. All values were normalized by equilibrating the expression of the ERCC probes between experiments as described previously (Loven et al., 2012). Briefly, normalize.loess from the affy R package was used to equilibrate the expression of ERCC probes between experiments and used to normalize the expression of RefSeq genes. A one-tailed Wilcoxon rank sum test of whether RefSeq gene levels decreased upon exosome knockdown showed they did not decrease.

Since expression of RefSeq genes—largely mRNAs—is not lost upon exosome knockdown, the RefSeq genes were used as a normalization method to determine if enhancer RNAs and promoter RNAs tended to increase upon exosome knockdown. FPKM values were calculated for RefSeq genes, ERCC probes, promoters of 16,202 non-overlapping RefSeq genes, and all constituent enhancers. These values were floored at 0.01 and a pseudocount of 0.1 was added. After normalizing using expressed RefSeq genes (RPKM >1 in both cases) as the constant, it was noted that levels of these non-coding RNAs increased. The amount of non-coding RNA level increase is conservative and low because the evidence suggests that mRNA levels also increase upon exosome knockdown. Thus, normalization shows that enhancer and promoter RNA levels become elevated even relative to the mRNA levels.

Metagene Analysis:

Enhancer metagenes were constructed using +/−2000 bases from the centers of regions co-bound by OCT4, SOX2, and NANOG in mouse ES cells as defined in (Whyte et al., 2013). Super-enhancer constituent metagenes were constructed using +/−2000 bases from the centers of super-enhancer constituents, which were defined as enhancers contacting super-enhancers as calculated in (Whyte et al., 2013). Promoter metagenes were constructed using +/−2000 bases from RefSeq transcription start sites that had no other transcription start sites in that window.

Densities were calculated in bins using bamToGFF (Lin et al., 2012). Resulting values were plotted in R using matplot. Figure panel-specific adjustments are noted in Table 2 below. Where noted, the top 20 most signal-rich regions were disregarded, because CLIP-seq signal was unusually concentrated therein, and all of these regions had at least one RepeatMasker-annotated repeat, often somewhat divergent RNA repeats. Where noted, “transcribed” promoters were used, which had GRO-seq RPM-normalized density >1. Where noted, “not transcribed” promoters were used, which had zero GRO-seq reads and zero H3K4me3 reads. Where noted, multiple reads with the same position were collapsed to one using samtools rmdup except in CLIP-seq analysis where reads were manually collapsed if they had the same barcode and position, and in motif analyses. Where noted, background signal was subtracted from ChIP or CLIP signal by either per-bin per-region normalization of counts in the exact same genomic location, or by subtracting the mean bin signals of input from the precipitated sample. The detailed description of the parameters used is shown in Table 2.

TABLE 2
Parameters for Metagene Analysis

| | | | | # | | | | |
|---|---|---|---|---|---|---|---|---|
| | | | Region | Regions | Region | | Positional | Background |
| FIG. | Factor | Treatment | Type | Used | Criteria | RPM | Duplicates | Normalization |
| 1B | GRO- | None | Promoters | 16202 | Non- | Y | Removed | None |
| | Seq | | | | Overlapping | | | |
| 1B | GRO- | None | Enhancers | 10627 | All | Y | Removed | None |
| | Seq | | | | | | | |
| 1D | Oct4 | None | Promoters | 16202 | Non- | Y | Removed | None |
| | ChIP- | | | | Overlapping | | | |
| | Seq | | | | | | | |
| 1D | Oct4 | None | Enhancers | 10627 | All | Y | Removed | None |
| | ChIP- | | | | | | | |
| | Seq | | | | | | | |
| 1D | YY1 | None | Promoters | 16202 | Non- | Y | Removed | None |
| | ChIP- | | | | Overlapping | | | |
| | Seq | | | | | | | |
| 1D | YY1 | None | Enhancers | 10627 | All | Y | Removed | None |
| | ChIP- | | | | | | | |
| | Seq | | | | | | | |
| 1D | YY1 | None | Promoters | 16182 | Non- | Y | Removed if | None |
| | CLIP- | | | | Overlapping, | | same | |
| | Seq | | | | Discard top 20 | | barcode | |
| 1D | YY1 | None | Enhancers | 10607 | Discard top 20 | Y | Removed if | None |
| | CLIP- | | | | | | same | |
| | Seq | | | | | | barcode | |
| 1D | Oct4 | None | Promoters | 16202 | Non- | N, no | Kept | None |
| | Motifs | | | | Overlapping | density | | |
| 1D | Oct4 | None | Enhancers | 10627 | All | N, no | Kept | None |
| | Motifs | | | | | density | | |
| 1D | YY1 | None | Promoters | 16202 | Non- | N, no | Kept | None |
| | Motifs | | | | Overlapping | density | | |
| 1D | YY1 | None | Enhancers | 10627 | All | N, no | Kept | None |
| | Motifs | | | | | density | | |
| 3B | GRO- | None | Promoters | 16202 | Non- | N | Removed | None |
| | Seq | | | | Overlapping | | | |
| 3B | GRO- | None | Enhancers | 10627 | All | N | Removed | None |
| | Seq | | | | | | | |
| 3B | GRO- | None | SE | 646 | All | N | Removed | None |
| | Seq | | Constituents | | | | | |
| 3B | GRO- | DRB | Promoters | 16202 | Non- | N | Removed | None |
| | Seq | | | | Overlapping | | | |
| 3B | GRO- | DRB | Enhancers | 10627 | All | N | Removed | None |
| | Seq | | | | | | | |
| 3B | GRO- | DRB | SE | 646 | All | N | Removed | None |
| | Seq | | Constituents | | | | | |
| 3B | YY1 | None | Promoters | 16202 | Non- | Y | Removed | Mean input |
| | ChIP- | | | | Overlapping | | | signal in each |
| | Seq | | | | | | | bin subtracted |
| 3B | YY1 | None | Enhancers | 10627 | All | Y | Removed | Mean input |
| | ChIP- | | | | | | | signal in each |
| | Seq | | | | | | | bin subtracted |
| 3B | YY1 | None | SE | 646 | All | Y | Removed | Mean input |
| | ChIP- | | Constituents | | | | | signal in each |
| | Seq | | | | | | | bin subtracted |
| 3B | YY1 | DRB | Promoters | 16202 | Non- | Y | Removed | Mean input |
| | ChIP- | | | | Overlapping | | | signal in each |
| | Seq | | | | | | | bin subtracted |
| 3B | YY1 | DRB | Enhancers | 10627 | All | Y | Removed | Mean input |
| | ChIP- | | | | | | | signal in each |
| | Seq | | | | | | | bin subtracted |
| 3B | YY1 | DRB | SE | 646 | All | Y | Removed | Mean input |
| | ChIP- | | Constituents | | | | | signal in each |
| | Seq | | | | | | | bin subtracted |
| 3C | GRO- | DRB −> | Promoters | 16202 | Non- | Y | Removed | None |
| | Seq | Release | | | Overlapping | | | |
| 3C | GRO- | DRB −> | Enhancers | 10627 | All | Y | Removed | None |
| | Seq | Release | | | | | | |
| 3C | GRO- | DRB −> | SE | 646 | All | Y | Removed | None |
| | Seq | Release | Constituents | | | | | |
| 3C | GRO- | DRB | Promoters | 16202 | Non- | Y | Removed | None |
| | Seq | | | | Overlapping | | | |
| 3C | GRO- | DRB | Enhancers | 10627 | All | Y | Removed | None |
| | Seq | | | | | | | |
| 3C | GRO- | DRB | SE | 646 | All | Y | Removed | None |
| | Seq | | Constituents | | | | | |
| 3C | YY1 | DRB −> | Promoters | 16202 | Non- | Y | Removed | Mean input |
| | ChIP- | Release | | | Overlapping | | | signal in each |
| | Seq | | | | | | | bin subtracted |
| 3C | YY1 | DRB −> | Enhancers | 10627 | All | Y | Removed | Mean input |
| | ChIP- | Release | | | | | | signal in each |
| | Seq | | | | | | | bin subtracted |
| 3C | YY1 | DRB −> | SE | 646 | All | Y | Removed | Mean input |
| | ChIP- | Release | Constituents | | | | | signal in each |
| | Seq | | | | | | | bin subtracted |
| 3C | YY1 | DRB | Promoters | 16202 | Non- | Y | Removed | Mean input |
| | ChIP- | | | | Overlapping | | | signal in each |
| | Seq | | | | | | | bin subtracted |
| 3C | YY1 | DRB | Enhancers | 10627 | All | Y | Removed | Mean input |
| | ChIP- | | | | | | | signal in each |
| | Seq | | | | | | | bin subtracted |
| 3C | YY1 | DRB | SE | 646 | All | Y | Removed | Mean input |
| | ChIP- | | Constituents | | | | | signal in each |
| | Seq | | | | | | | bin subtracted |
| 3D | YY1 | shLuc | Promoters | 16202 | Non- | Y | Removed | per-bin per- |
| | ChIP- | | | | Overlapping | | | region input |
| | Seq | | | | | | | subtraction |
| 3D | YY1 | shLuc | Enhancers | 10627 | All | Y | Removed | per-bin per- |
| | ChIP- | | | | | | | region input |
| | Seq | | | | | | | subtraction |
| 3D | YY1 | shLuc | SE | 646 | All | Y | Removed | per-bin per- |
| | ChIP- | | Constituents | | | | | region input |
| | Seq | | | | | | | subtraction |
| 3D | YY1 | shEXO10 | Promoters | 16202 | Non- | Y | Removed | per-bin per- |
| | ChIP- | | | | Overlapping | | | region input |
| | Seq | | | | | | | subtraction |
| 3D | YY1 | shEXO10 | Enhancers | 10627 | All | Y | Removed | per-bin per- |
| | ChIP- | | | | | | | region input |
| | Seq | | | | | | | subtraction |
| 3D | YY1 | shEXO10 | SE | 646 | All | Y | Removed | per-bin per- |
| | ChIP- | | Constituents | | | | | region input |
| | Seq | | | | | | | subtraction |
| SF1 | GRO- | None | Promoters | 9709 | Transcribed | Y | Removed | None |
| | Seq | | | | | | | |
| SF1 | GRO- | None | Promoters | 1118 | Not | Y | Removed | None |
| | Seq | | | | transcribed | | | |
| SF1 | OCT4 | None | Promoters | 9709 | Transcribed | Y | Removed | None |
| | ChIP- | | | | | | | |
| | Seq | | | | | | | |
| SF1 | OCT4 | None | Promoters | 1118 | Not | Y | Removed | None |
| | ChIP- | | | | transcribed | | | |
| | Seq | | | | | | | |
| SF1 | YY1 | None | Promoters | 9709 | Transcribed | Y | Removed | None |
| | ChIP- | | | | | | | |
| | Seq | | | | | | | |
| SF1 | YY1 | None | Promoters | 1118 | Not | Y | Removed | None |
| | ChIP- | | | | transcribed | | | |
| | Seq | | | | | | | |
| SF1 | YY1 | None | Promoters | 9709 | Transcribed | Y | Removed | None |
| | CLIP- | | | | | | | |
| | Seq | | | | | | | |
| SF1 | YY1 | None | Promoters | 1118 | Not | Y | Removed | None |
| | CLIP- | | | | transcribed | | | |
| | Seq | | | | | | | |
| SF2 | Oct4 | None | Promoters | 13870 | All plus | N, no | Kept | None |
| | Motifs | | | | strand | density | | |
| SF2 | Oct4 | None | Promoters | 13728 | All minus | N, no | Kept | None |
| | Motifs | | | | strand | density | | |
| SF2 | Oct4 | None | Enhancers | 10627 | All | N, no | Kept | None |
| | Motifs | | | | | density | | |
| SF2 | YY1 | None | Promoters | 13870 | All plus | N, no | Kept | None |
| | Motifs | | | | strand | density | | |
| SF2 | YY1 | None | Promoters | 13728 | All minus | N, no | Kept | None |
| | Motifs | | | | strand | density | | |
| SF2 | YY1 | None | Enhancers | 10627 | All | N, no | Kept | None |
| | Motifs | | | | | density | | |
| SF4 | YY1 | None | Promoters | 27598 | All | Y | Kept | None |
| | CLIP- | | | | | | | |
| | Seq | | | | | | | |
| SF4 | YY1 | None | Promoters | 27598 | All | Y | Kept | None |
| | CLIP- | | | | | | | |
| | Seq | | | | | | | |
| SF5 | YY1 | None | Promoters | 16182 | Discard top 20 | Y | Removed if | Ctrl CLIP |
| | CLIP | | | | | | same | |
| | | | | | | | barcode | |
| SF5 | YY1 | None | Enhancers | 10607 | Discard top 20 | Y | Removed if | Ctrl CLIP |
| | CLIP | | | | | | same | |
| | | | | | | | barcode | |
| SF5 | Oct4 | None | Promoters | 16182 | Discard top 20 | Y | Removed if | Ctrl CLIP |
| | CLIP | | | | | | same | |
| | | | | | | | barcode | |
| SF5 | Oct4 | None | Enhancers | 10607 | Discard top 20 | Y | Removed if | Ctrl CLIP |
| | CLIP | | | | | | same | |
| | | | | | | | barcode | |
| SF9B | GRO- | None | Promoters | 16202 | Non- | Y | Removed | None |
| | Seq | | | | Overlapping | | | |
| SF9B | GRO- | None | Enhancers | 10627 | All | Y | Removed | None |
| | Seq | | | | | | | |
| SF9B | GRO- | None | SE | 646 | All | Y | Removed | None |
| | Seq | | Constituents | | | | | |
| SF9B | GRO- | ActD | Promoters | 16202 | Non- | Y | Removed | None |
| | Seq | | | | Overlapping | | | |
| SF9B | GRO- | ActD | Enhancers | 10627 | All | Y | Removed | None |
| | Seq | | | | | | | |
| SF9B | GRO- | ActD | SE | 646 | All | Y | Removed | None |
| | Seq | | Constituents | | | | | |
| SF9B | YY1 | None | Promoters | 16202 | Non- | Y | Removed | Mean input |
| | ChIP- | | | | Overlapping | | | signal in each |
| | Seq | | | | | | | bin subtracted |
| SF9B | YY1 | None | Enhancers | 10627 | All | Y | Removed | Mean input |
| | ChIP- | | | | | | | signal in each |
| | Seq | | | | | | | bin subtracted |
| SF9B | YY1 | None | SE | 646 | All | Y | Removed | Mean input |
| | ChIP- | | Constituents | | | | | signal in each |
| | Seq | | | | | | | bin subtracted |
| SF9B | YY1 | ActD | Promoters | 16202 | Non- | Y | Removed | Mean input |
| | ChIP- | | | | Overlapping | | | signal in each |
| | Seq | | | | | | | bin subtracted |
| SF9B | YY1 | ActD | Enhancers | 10627 | All | Y | Removed | Mean input |
| | ChIP- | | | | | | | signal in each |
| | Seq | | | | | | | bin subtracted |
| SF9B | YY1 | ActD | SE | 646 | All | Y | Removed | Mean input |
| | ChIP- | | Constituents | | | | | signal in each |
| | Seq | | | | | | | bin subtracted |
| SF9C | YY1 | None | Promoters | 16202 | Non- | Y | Removed | Mean input |
| | ChIP- | | | | Overlapping | | | signal in each |
| | Seq | | | | | | | bin subtracted |
| SF9C | YY1 | None | Enhancers | 10627 | All | Y | Removed | Mean input |
| | ChIP- | | | | | | | signal in each |
| | Seq | | | | | | | bin subtracted |
| SF9C | YY1 | None | SE | 646 | All | Y | Removed | Mean input |
| | ChIP- | | Constituents | | | | | signal in each |
| | Seq | | | | | | | bin subtracted |
| SF9C | YY1 | THZ1 | Promoters | 16202 | Non- | Y | Removed | Mean input |
| | ChIP- | | | | Overlapping | | | signal in each |
| | Seq | | | | | | | bin subtracted |
| SF9C | YY1 | THZ1 | Enhancers | 10627 | All | Y | Removed | Mean input |
| | ChIP- | | | | | | | signal in each |
| | Seq | | | | | | | bin subtracted |
| SF9C | YY1 | THZ1 | SE | 646 | All | Y | Removed | Mean input |
| | ChIP- | | Constituents | | | | | signal in each |
| | Seq | | | | | | | bin subtracted |
| SF9C | YY1 | TPI | Promoters | 16202 | Non- | Y | Removed | Mean input |
| | ChIP- | | | | Overlapping | | | signal in each |
| | Seq | | | | | | | bin subtracted |
| SF9C | YY1 | TPI | Enhancers | 10627 | All | Y | Removed | Mean input |
| | ChIP- | | | | | | | signal in each |
| | Seq | | | | | | | bin subtracted |
| SF9C | YY1 | TPI | SE | 646 | All | Y | Removed | Mean input |
| | ChIP- | | Constituents | | | | | signal in each |
| | Seq | | | | | | | bin subtracted |

Statistical Tests for Change in Binding:

p-values were calculated by comparing the distributions of values of drug-treated and control sequencing samples using one-tailed Student's t-test unless otherwise noted. Values were calculated using one value per sample for the whole displayed region.

Heatmap Analysis:

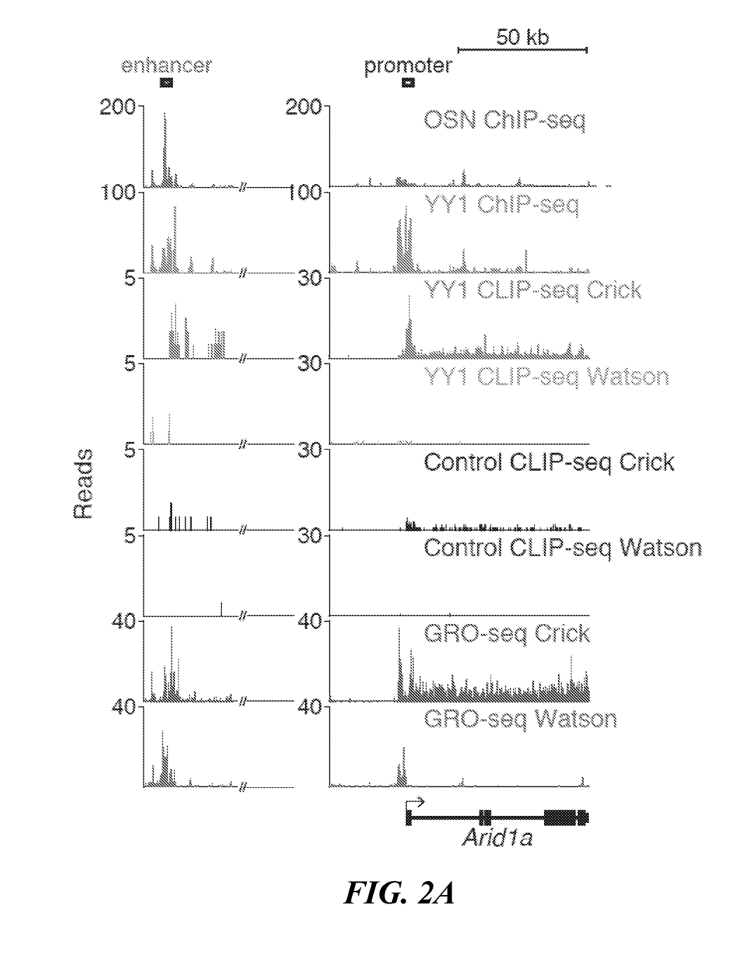

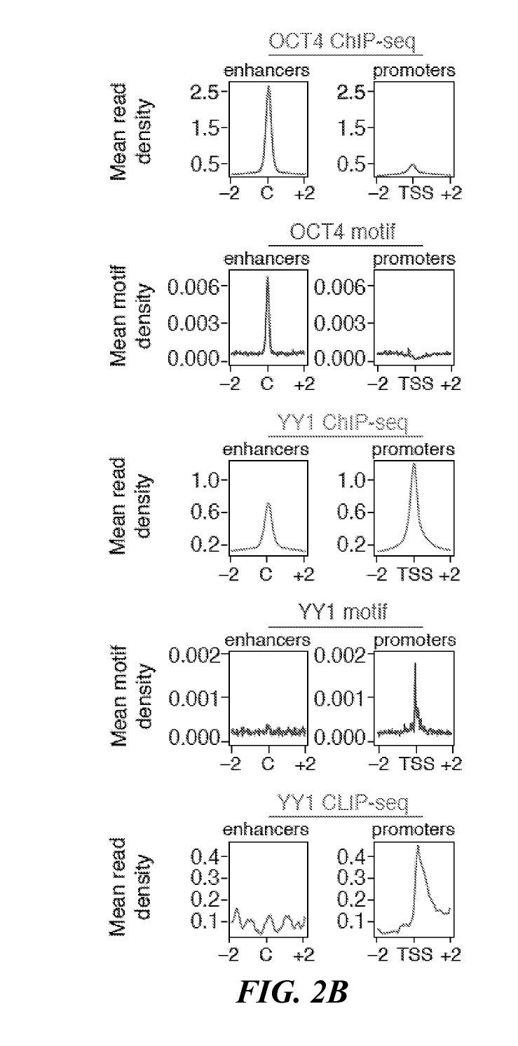

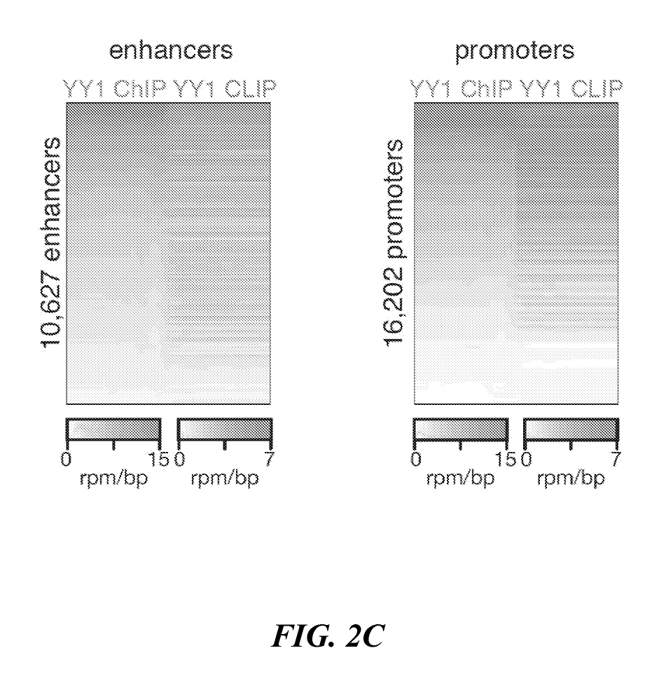

16,202 promoters (described above) and 10,627 enhancers (described above) were used for heatmap analysis in FIG. 2C. RPM-normalized read densities per region were calculated using bamToGFF (-d -r). Regions are ordered by ChIP-seq values and visualized using heatmap.2 in R.

Read Distribution Analysis:

For FIG. 5, reads were categorized by the type of region they fell into: enhancer (+/−2 kb around the center of 10,627 OSN sites, described above), promoter (+/−2 kb around 16,202 filtered RefSeq transcription start site, described above), RefSeq intron, RefSeq exon, other. Reads that fell in multiple categories of DNA regions were sorted into the most former category on this list. Reads were categorized using bedtools intersect (Quinlan and Hall, 2010) and the amount of genome in each category was calculated using bedtools subtract. To normalize read counts, the number of reads falling in a category of region was divided by the total number of DNA bases in all regions of that type.

Global Run-on Sequencing (GRO-Seq) Sample Preparation:

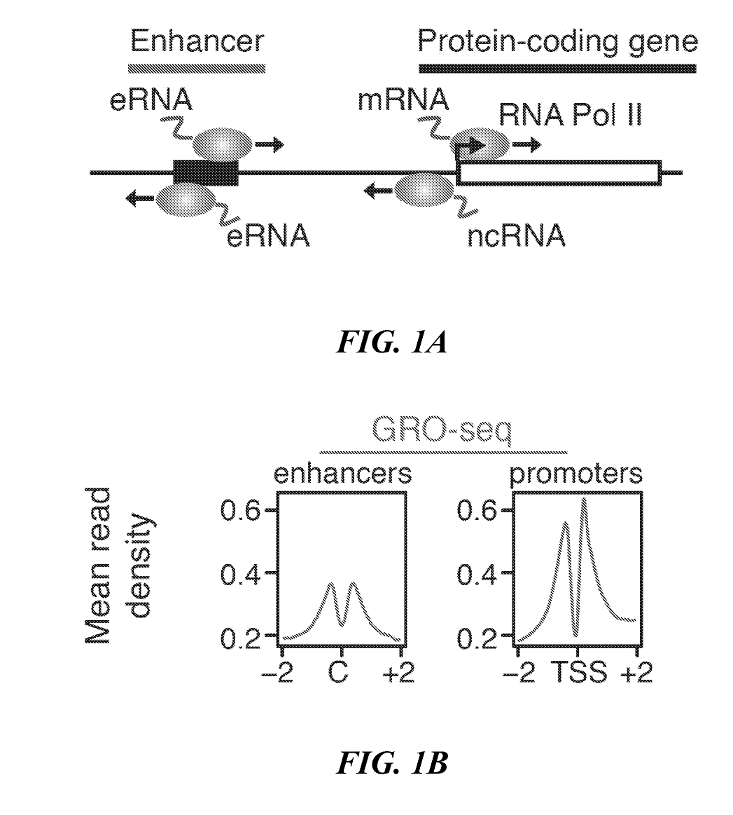

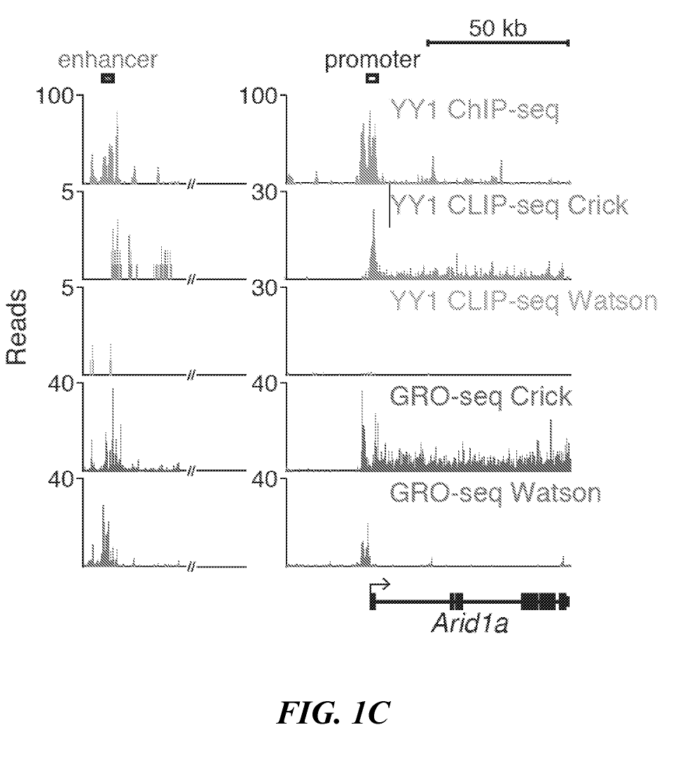

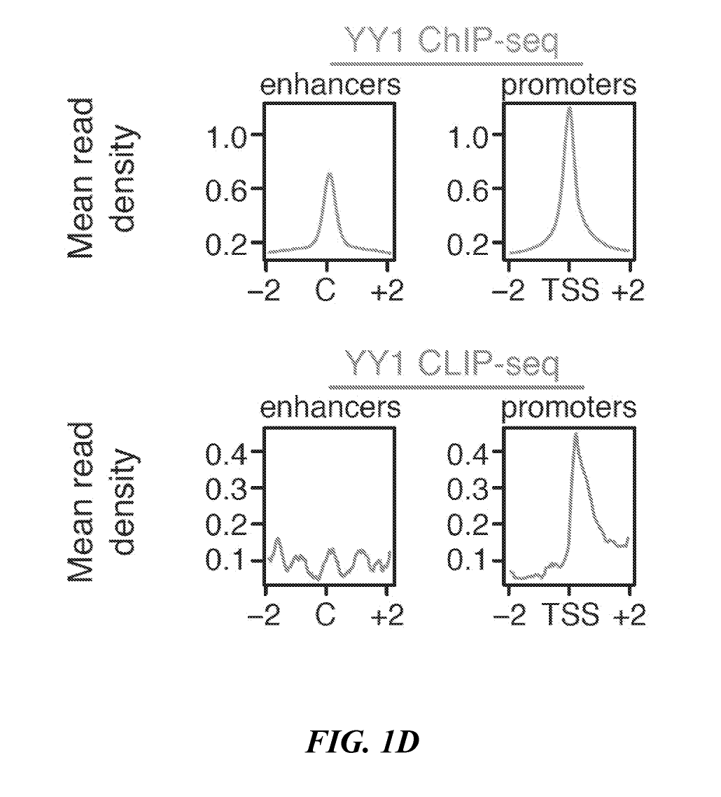

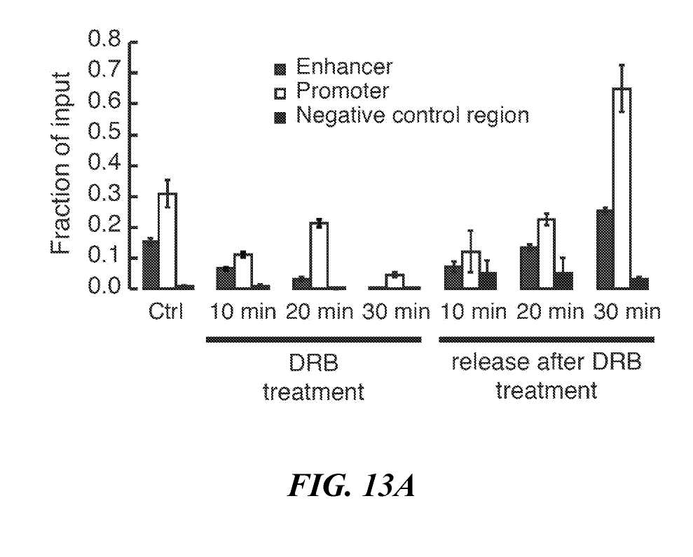

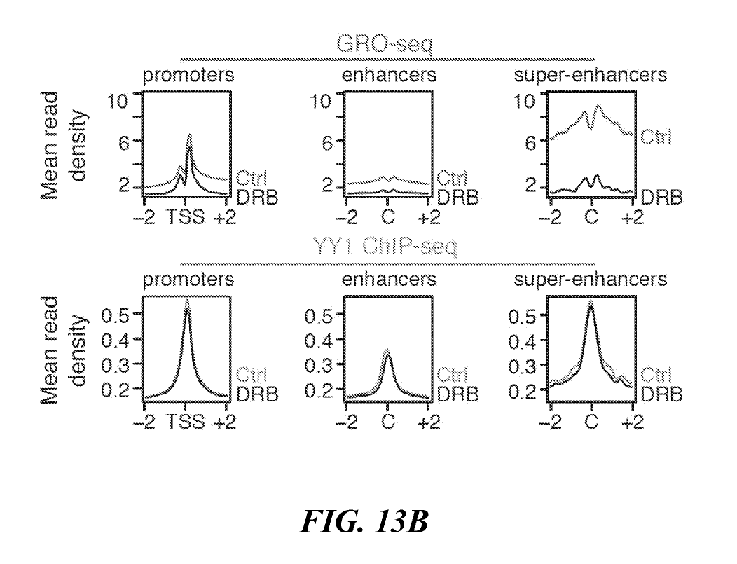

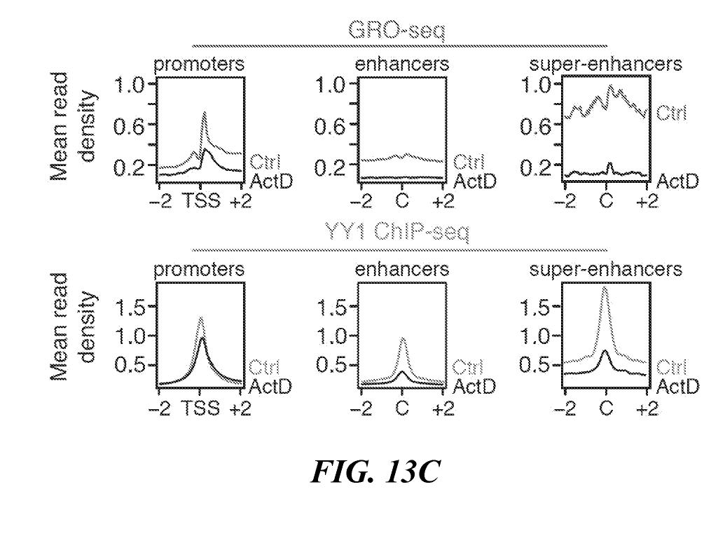

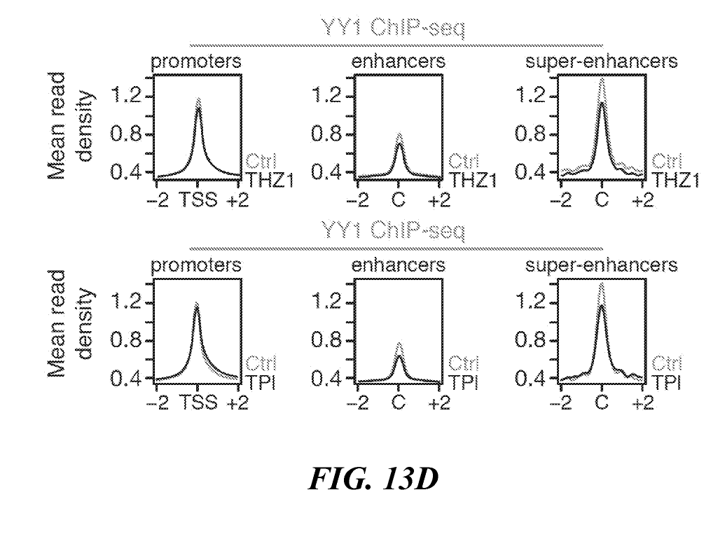

Gro-seq analysis for FIG. 1B, FIG. 1C, and FIG. 2A was performed as described in (Sigova et al., 2013). Gro-seq for FIG. 13C was performed as described previously (Wang et al., 2011) by using 1×10⁸bio-YY1 mESCs. Briefly, the nuclei were isolated first, and then in vitro nuclear run-on assay was performed with Br-UTP. The nascent RNAs were labeled with Br-UTP, these RNAs were hydrolysis by NaOH and purified by anti-BrdU argarose beads (Santa Cruz Biotech). The purified nascent RNAs were then phosphorylated, added poly A at 3′ end, and reverse transcribed into cDNA. The cDNAs were purified through gel extraction, and circularized by CircLigase. The circular single-stranded DNA was linearized by APE1 endonuclease, and PCR amplified. The final PCR product was purified and sequenced on the Illumina HiSeq 2000.

Global Run-on Sequencing (GRO-Seq) Analysis:

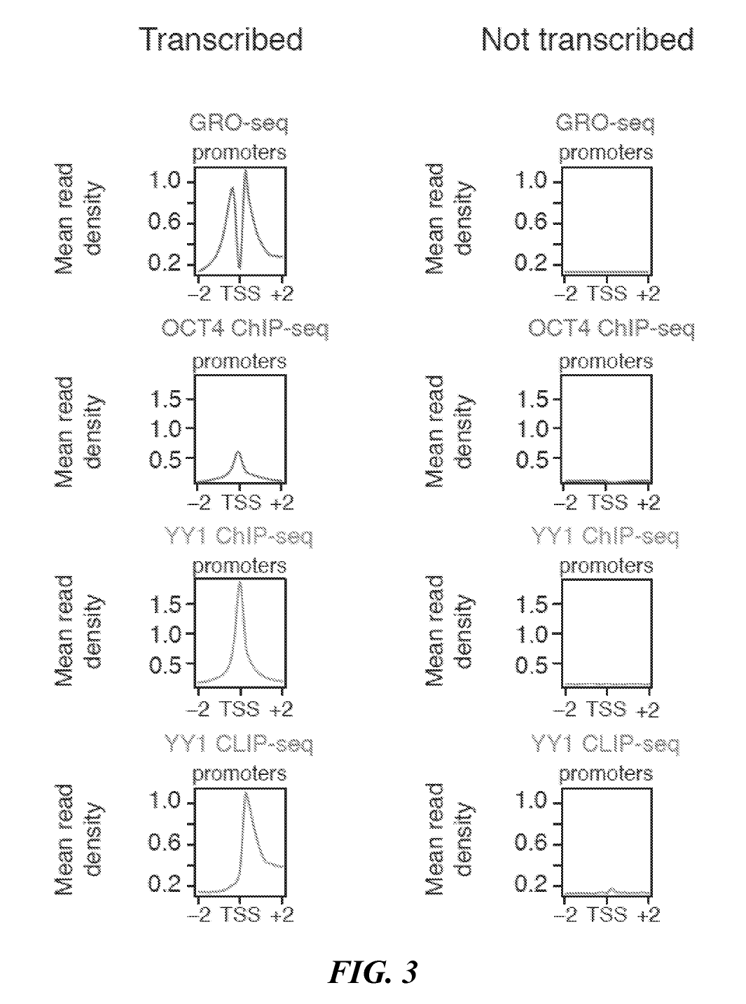

Once sequenced, the 40 bp GRO-seq reads used in FIG. 1C and FIG. 2A were first filtered off ribosomal RNAs using SortmeRNA (Kopylova et al., 2012) with the eukaryote rRNA databases to account for rRNA genes in the proximity of Arid1a. Reads used in metagenes in FIG. 1B and FIG. 3 were not filtered in this manner. Both filtered and unfiltered reads were then trimmed at their 3′ ends to remove residual sequencing adaptor sequences, and trimmed reads over 24 bp were aligned to the mm9 version of the mouse genome using Bowtie 0.12.9 (Langmead et al., 2009) as described in (Sigova et al., 2013). Reads having a Phred score quality under 33 or aligning to more than 10 locations in the genome were discarded, and only the best mapping location was retained for each read. A high number of reads mapping to chr17:39979942-39985774+strand region encoding a 45S rRNA precursor not entirely contained in the eukaryote rRNA database were additionally removed. Reads aligning to the + or − strand of the genome were respectively separated into Watson and Crick strand read containing files and wiggle tracks were generated for each file using bedtools (Quinlan and Hall, 2010). Wig format wiggle files were converted into BigWig format with wigToBigWig (Kent et al., 2010) and uploaded for visualization in the UCSC genome browser (Kent et al., 2002).

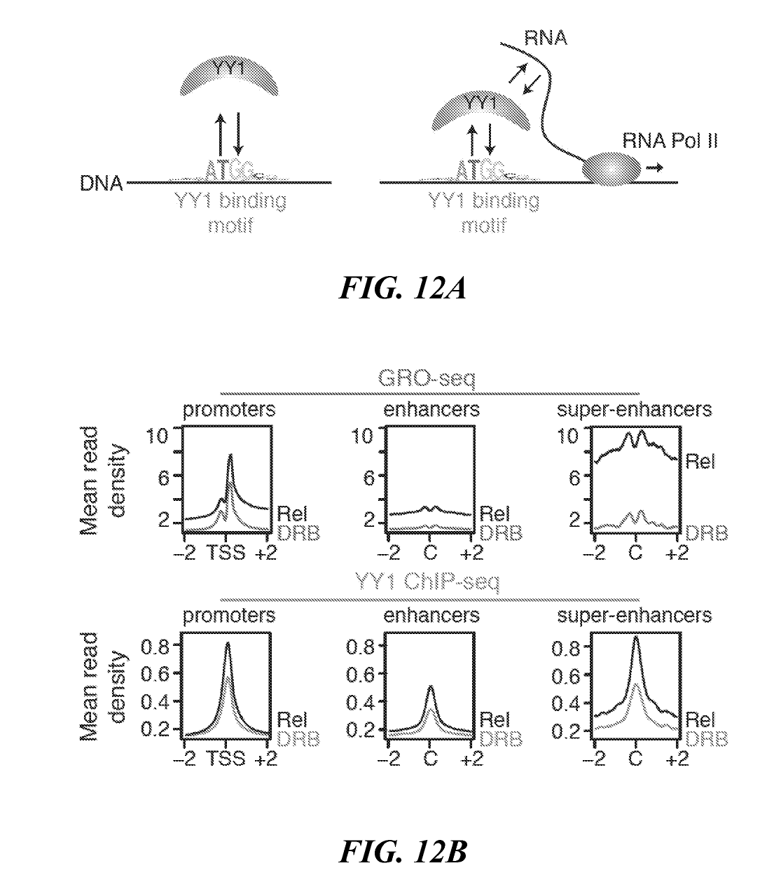

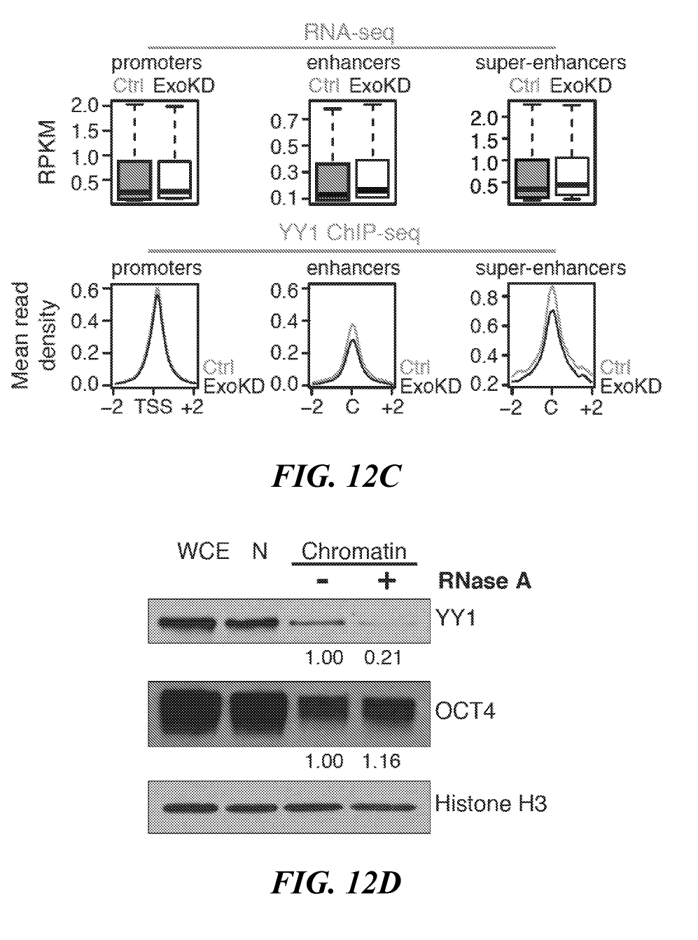

Untreated and ActD-treated GRO-seq reads in FIG. 13C were trimmed at the 3′ end at the sequence TGGAATTCTCGGGTGCCAAGGAACTCCAGTCAC (SEQ ID NO: 7) using cutadapt -m18. Trimmed reads were aligned with bowtie with parameters -p 20 -n 2 -S -k 1 -m 1*best and these reads were discarded in favor of those that had polyA tails. PolyA tails for unmapped reads were trimmed using prinseq -trim_tail_right 1 (Schmieder and Edwards, 2011). PolyA-trimmed reads were aligned using bowtie -n 2 -S -k 1 -m 1 -best to retain reads that could be uniquely mapped. GRO-seq reads used in FIG. 12B and FIG. 13B are from Wang et al., 2015.

Affinity Purification and Sequencing of YY1-Associated RNA (YY1 CLIP-Seq):

CLIP-seq was performed as previously described (Jangi et al., 2014) with the following modifications. Bio-YY1 ESCs and control ESCs expressing only biotin ligase BirA were grown in 15-cm plates. Each plate was washed twice with 30 ml of ice-cold PBS, and UV-crosslinking was conducted with 254 nm UV light when plates were placed on ice with lids off at 400 mJ/cm2. Cells were scraped off each plate and collected in 15-ml Falcon tubes. Nuclei were purified using Nuclei EZ Prep nuclei isolation kit (Sigma) and flash frozen until ready to use. Three 15-cm plates (˜1.5×10⁸ cells) of ESCs were used for each experiment. 50 μl of Dynabeads® MyOne Streptavidin T1 was used for each 0.5×10⁸ cells. Beads were washed thrice with PBS, twice with DEPC-treated 0.1M NaOH, 0.05M NaCl, and once with DEPC-treated 0.1M NaCl. After blocking the beads with sterile-filtered blocking buffer (0.5% BSA in PBS) for 30 min at 4° C., beads were kept on ice in 100 μl of the lysis buffer (1×PBS, 0.1% SDS, 0.5% sodium deoxycholate, 0.5% NP-40, 1 mM DTT, cOmplete EDTA-free protease inhibitor cocktail (Roche)) until ready to use.

Each aliquot of 0.5×10⁸ cells was thawed on ice and resuspended in 1 ml of the lysis buffer in 1.5 ml tubes. Cells were passed three times through the 26″ needle to reduce viscosity, and tubes were rotated for 10 min at 4° C. to complete the lysis. 30 μl of RQ1 RNase-free DNAse (Promega) and 1 μl of RNAse I (Life Technologies, 100U/μl) were added to each 1 ml of lysate and tubes were incubated at 37° C. for 15 min in the ThermoMixer C (Eppendorf) (rock for 15 sec at 1,200 rpm, 75 sec off). RNase digestion was stopped by transferring the tubes to ice and adding 25 μl SuperaseIn RNase Inhibitor (Life Technologies, 20U/μl) to each aliquot of 0.5×108 cells. Lysates were cleared up by centrifugation at 20,000 g for 10 min at 4° C., supernatants were added to the beads, and rotated overnight at 4° C. 2% of the supernatant was saved as input for Western blot analysis.

At the end of incubation, beads were washed twice by rotating them for 5 min each time with 1 ml of ice-cold wash buffer 1 containing 50 mM Tris-HCl, pH 7.4, 1 mM EDTA, 1M NaCl, 0.1% SDS, 1% NP-40, 1 mM DTT, 10 u/ml SuperaseIN, and cOmplete protease inhibitor followed by two washes (each for 5 min at 4° C.) with 1 ml of wash buffer 2 containing 50 mM Tris-HCl, pH 7.4, 1 mM EDTA, 300 mM NaCl, 0.1% SDS, 1% NP-40, 1 mM DTT, 10 u/ml SuperaseIN, and cOmplete protease inhibitor. The second DNase-treatment was conducted by incubating beads in 50-μl reaction in presence of 5 μl of 10× TurboDNAse buffer and 2 μl of TurboDNase (2 u/μl) (Life Technologies) for 25 min at 37° C. in the ThermoMixer C (rock for 15 sec at 1,000 rpm, 90 sec off). Beads were washed twice by rotating them for 5 min each time with 1 ml of ice-cold wash buffer 1 containing 50 mM Tris-HCl, pH 7.4, 1 mM EDTA, 500 mM NaCl, 0.1% SDS, 1% NP-40, 1 mM DTT, 10 u/ml SuperaseIN, and cOmplete protease inhibitor followed by two washes with 1 ml of wash buffer 2, two washes with 1 ml of RIPA-S buffer containing 50 mM Tris-HCl pH 7.4, 1M NaCl, 2M Urea, 0.5% NP-40, 1% sodium deoxycholate, 5 mM EDTA, 0.1% SDS, 1 mM DTT, and two washes with 1 ml of PNK buffer containing 50 mM Tris-HCl pH 7.4, 10 mM MgCl2, and 0.5% NP-40. Beads were resuspended in 1000 μl of PNK buffer and 100 μl of the beads was transferred to a clean tube. After removal of the PNK buffer, these beads were resuspended in 30 μl of PNK labeling mix containing 3 μl of 10×PNK buffer (NEB), 1 μl of T4 PNK (NEB) and 1 μl of ATP, g-³²P (Perkin Elmer, NEG035C001MC) and incubated for 20 min at 37° C. in the ThermoMixer C (rock for 15 sec at 1,000 rpm, 90 sec off). Labeling mix was removed and beads were washed three times with 50 mM Tris-HCl, pH 7.4, 1 mM EDTA, 150 mM NaCl, 0.1% SDS, 1% NP-40, 1 mM DTT. Unlabeled 900-μl worth of beads was washed in the same manner. Labeled RNA species cross-linked to YY1 were eluted off beads by incubating beads for 10 min at 95° C. in 20 μl of 1× NuPage LDS sample buffer (Life technologies) in presence of 10 mM DTT. Of the remaining 900 μl of unlabeled beads, 100 μl was used for Western blot analysis, and protein-RNA complexes were eluted off the remaining beads in 40 μl of 1× NuPage LDS sample buffer, 10 mM DTT. Protein-RNA complexes were separated on a 10% Criterion XT Bis-Tris gel (Bio-Rad) and transferred to nitrocellulose membrane (Bio-Rad) for 2 hrs (250 mA) at 4° C. Samples for Western blot analysis were resolved on SDS-PAGE and transferred to another membrane in parallel with the CLIP samples. After transfer, membrane was rinsed with TBS buffer, wrapped in Saran Wrap and exposed to film. Regions slightly above the radioactive band corresponding to YY1 protein based on results of the Western blot analysis, were excised from the membrane, sliced into thin strips, and transferred to 2 ml tubes. RNA was isolated in the following way: 400 μl of proteinase K buffer containing 100 mM Tris-HCl pH 7.4, 50 mM NaCl, 10 mM EDTA and 4 mg/ml proteinase K (20 mg/ml) (Life Technologies) was added to the tubes, which were rocked for 40 min at 37° C. in the ThermoMixer C at 1,200 rpm. Once 400 μl of PKU buffer containing 100 mM Tris-HCl pH 7.4, 50 mM NaCl, 10 mM EDTA, 7M Urea was added to the tubes, they were incubated for another 40 min at 37° C. in the ThermoMixer C at 1,200 rpm. Liquid fraction was transferred to a fresh 2 ml tube, 800 μl of phenol:chlorophorm:isoamyl alcohol (25:24:1 v/v) pH 8.0 (Sigma) was mixed with it and loaded to MaxTract High Density tubes (Quagen). After centrifugation at 16,000 g for 5 min, aqueous phase was transferred to two clean tubes and RNA was precipitated in presence of 0.3 M sodium acetate, 1 μl of GlygoBlue (Life Technologies) and 3× volume of 100% ethanol at −20° C. overnight.

Precipitated RNA pellets were washed with 70% ethanol and dried. At this point, UV-crosslinked RNA purified from all 1.5×10⁸ cells was pulled from all six tubes and resuspended in 10 μl of RNase-free H₂O. RNA was dephosphorylated in a 20-μl reaction containing 70 mM Tris-HCl pH 6.5, 10 mM MgCl₂, 5 mM DTT, 0.5 μl SuperaseIN, and 2 μl PNK (NEB) at 37° C. for 1 hr. After phenol/chloroform extraction, RNA was ethanol precipitated and resuspended in 13 μl H₂O. RNA was denatured by incubation at 70° C. for 2 min and kept on ice until ready to use. Subsequently, adapter with sequence /5rApp/TG GAA TTC TCG GGT GCC AAG G/3ddC/ (SEQ ID NO: 8) was ligated to the 3′ end of the RNA in a 20 μl reaction containing 2004 adapter, 1×RNA ligase buffer, 2 μl PEG 8000, and 2 μl truncated RNA ligase (NEB, M0373) at 16° C. overnight. Reactions were ethanol precipitated and reverse transcription of the RNA was conducted using SuperScript III reverse transcriptase (Life Technologies) at 55° C. for 1 hr using RT primer [Phos]ANNNNagatcGGAAGAGCGTCGTGTAGGGAAAGAGTGT[Sp-C18]CACTCA[Sp-C18]CCTTGGCACCCGAGAATTCCA (SEQ ID NO: 9). Subsequently, RNA was removed by treating the sample with 1 μl RNase H and 1 μl (0.3 mg/ml) RNase A at 37° C. for 20 min. After phenol/chloroform extraction, cDNA was ethanol precipitated and resuspended in 10 μl H2O.

cDNA denatured in presence of gel loading dye II (Life Technologies) at 70° C. for 3 min was separated from the adapter and the RT primer on a 10% TBE-Urea polyacrylamide gel (10 Watts, 1.5-2 hrs). Gel was stained with SYBR Gold Nucleic Acid Stain (Life Technologies) and area corresponding to 100-200 nt was excised from the gel. Excised gel fragments were further fragmented and cDNA was eluted in buffer containing 0.5M NaCl and 1 mM EDTA by rotating tubes at room temperature overnight. Gel slurry was transferred to a Costar Spin-X 0.22 μm column (Corning, 8161) and spun at 8,000 g for 3 min. cDNA was consequently ethanol precipitated, resuspended in 8 μl H₂O, and circularized in presence of 1 μl CircLigase II buffer, 0.5 μl MnCl₂ and 0.5 μl CircLigasell (Epicentre, CL9021K) by incubating the cDNA at 60° C. for 1 hr. After CircLigaseII heat inactivation at 80° C. for 10 min, DNA was ethanol precipitated and resuspended in 21 μl H₂O. 5 μl of the DNA was used as a template in each of two 50-μl PCR reactions using Phusion High Fidelity DNA polymerase (NEB, M0530) (7 cycles, empirically determined) and the following primers:

DSFP5
(SEQ ID NO: 10)
(Aatgatacggcgaccaccgagatctacactctttccctacacgacgct
cttcc)
and
the following bar-coded primers:
DSFP3_1 for control BirA sample
(SEQ ID NO: 11)
CAAGCAGAAGACGGCATACGAGATCGTGATCGGTCCTTGGCACCCGAGA
ATTCCA
DSFP3_2 for bio-YY1 sample
(SEQ ID NO: 12)
CAAGCAGAAGACGGCATACGAGATACATCGCGGTCCTTGGCACCCGAGAA
TTCCA

PCR products were ethanol precipitated and separated on a 10% TBE-Urea polyacrylamide gel. After gel staining with SYBR Gold, 150-250 nt gel fragments were excised from a gel and DNA was eluted by incubating gel pieces in 0.5M NaCl and 1 mM EDTA by rotating tubes at room temperature overnight. DNA was ethanol precipitated and resuspended in 10 μl H₂O. After diluting the DNA ten-fold, 1 μl of the diluted DNA was used as a template in the next round of 15 cycles of PCR for bio-YY1 sample and 25 cycles of PCR for control BirA sample using either Phusion High-Fidelity DNA polymerase or GoTaq DNA polymerase (Promega) and the following primers: AATGATACGGCGACCACC (SEQ ID NO: 13) and CAAGCAGAAGACGGCATAC (SEQ ID NO: 14). PCR products were resolved on 2% Agarose Resolute GPG gel (American Bioanalytical), isolated from a gel, and PCR products synthesized using GoTaq DNA polymerase was cloned into TOPO-TA vector (Life Technologies) and resulting plasmid was subjected to Sanger sequencing to confirm complexity of the insert. PCR product obtained using Phusion High Fidelity DNA polymerase was subjected to high-throughput sequencing on Illumina High-Seq genome analyzer. Insert sequencing primer was CTCTTTCCCTACACGACGCTCTTCCGATCT (SEQ ID NO: 15) and barcode sequencing primer was TGGAATTCTCGGGTGCCAAGGACCG (SEQ ID NO: 16).

OCT4 CLIP-seq was conducted similarly to YY1 CLIP-seq using bio-OCT4 mESCs described in (Kim et al., 2008).

YY1 CLIP-Seq Analysis:

CLIP-Seq reads were processed in a manner adapted from (Jangi et al., 2014). Cutadapt (Martin, 2011) was used to trim the sequence TGGAATTCTCGGGTGCCAAGG off of the 3′ end of CLIP-Seq reads with parameter -m 18. First, bowtie was used to map trimmed reads to the mm9 mouse genome with parameters -best -strata -m1 -n 2-5 5 -p 50 -S. Bowtie2 (Langmead and Salzberg, 2012) was used to map those reads bowtie failed to map (accounting for deletions caused by UV crosslinking) with parameters -5 5 -sensitive -p 50. The resulting bowtie and bowtie2 mappings were combined and used for downstream analysis.

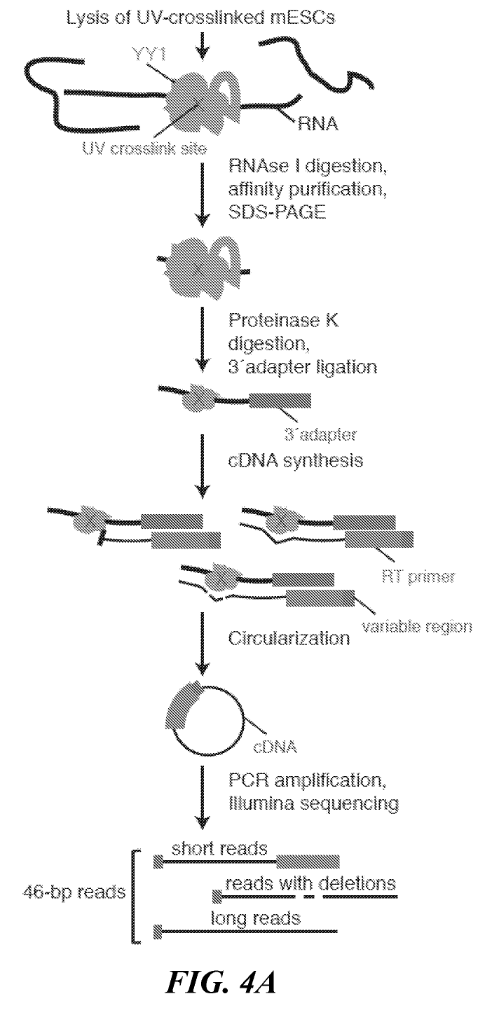

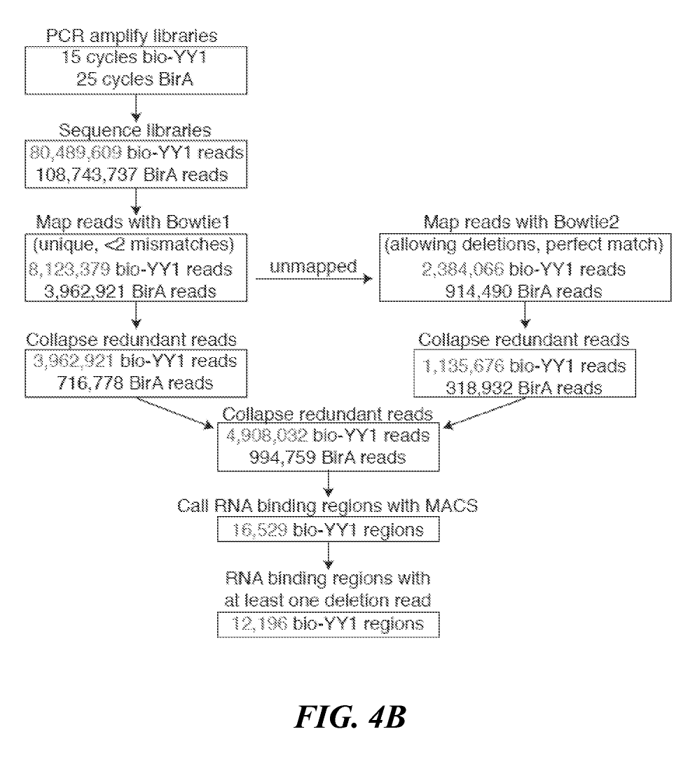

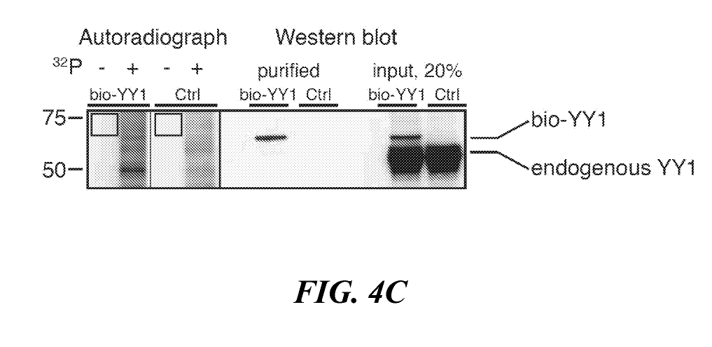

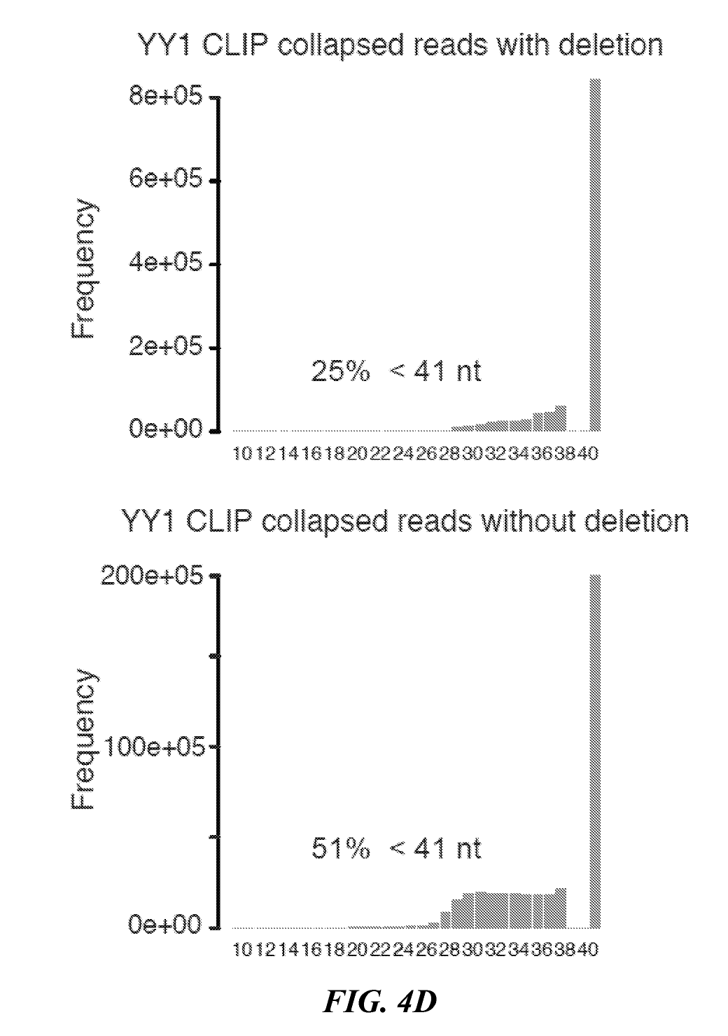

Multiple reads were collapsed to one if they had the same chromosome, start, end, and first four nucleotides (barcode) to account for PCR duplicates. RepeatMasker regions for mm9 were downloaded from the UCSC Genome Browser and subsetted for rRNA repeats. Reads contacting these regions were discarded. Remaining reads were separated into + and − strand reads using samtools view (Li et al., 2009). Enriched regions and WIG files were created on strand-separated mapping files using MACS with parameters -w -S -space=50 -nomodel -shiftsize=40 -keep-dup=1. Peaks identified separately on reads mapping to each strand were concatenated and used for FIG. 4. Peaks were required to have at least one read mapped with bowtie2.

Purification and Determination of Concentration of the Murine Recombinant YY1:

Murine YY1 protein was purified using a method modified from (Jeon and Lee, 2011). Briefly, a plasmid containing the N-terminal His₆-tagged YY1 coding sequence (a gift from Dr. Yang Shi) was transformed into BL21-CodonPlus (DE3)-RIL cells (Stratagene, 230245). A fresh bacterial colony was inoculated into LB media containing ampicillin and chloramphenicol and grown overnight at 37° C. Bacteria were pelleted, resuspended in 500 ml of fresh pre-warmed LB media, and grown for 1.5 hours at 37° C. After induction of YY1 expression with 1 mM IPTG, cells were grown for another 5 hours, collected, and stored frozen at −80° C. until ready to use.

Pellets were resuspended in 5 ml of Buffer A (6M GuHCL, 25 mM Tris, 100 mM NaCl, pH8.0) containing 10 mM imidazole, 5 mM 2-mercaptoethanol, and cOmplete protease inhibitors (Roche) and sonicated (ten cycles of 15 seconds on, 60 sec off). The lysate was cleared by centrifugation at 13,000 g for 20 minutes at 4° C., 1 ml of Ni-NTA agarose (Invitrogen, R901-15) pre-equilibrated with 10× volumes of buffer A was added to the cleared lysate, and tubes containing agarose lysate slurry were rotated at room temperature for 1 hour. The agarose slurry was poured into a column, and packaged agarose was washed with 15× volumes of Buffer A containing 10 mM imidazole and 5 mM DTT. Protein was eluted with 3 ml Buffer A containing 500 mM imidazole and 5 mM DTT.

After adjusting the concentration of ZnCl₂ and DTT in the elution buffer to 0.1 mM and 100 mM, respectively, the purified protein was denatured at 60° C. for 30 minutes and refolded by dialyzing it against 600 ml of the dialysis buffer containing 25 mM Tris-HCl pH 8.5, 100 mM NaCl, 10 mM MgCl₂, 0.1 mM ZnCl₂, and 5 mM DTT at 4° C. changing the buffer three times. At the end of the dialysis, precipitated material was removed by centrifugation at 1,800 g for 15 minutes at 4° C. Soluble fraction was dialyzed against 600 ml of the dialysis buffer containing 1 mM DTT and 10% glycerol at 4° C. changing the buffer three times. At the end of the dialysis, protein was stored in aliquots at −80° C.

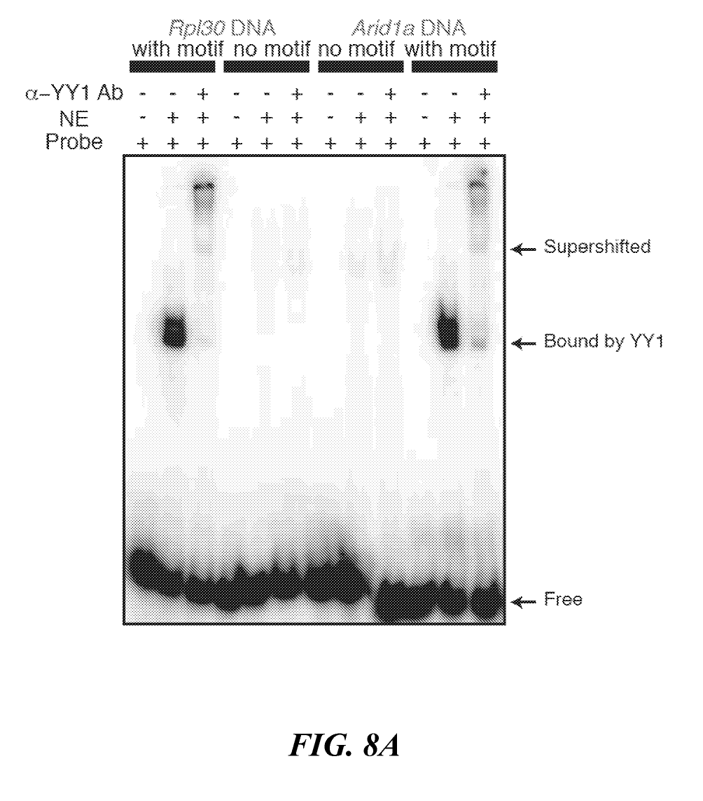

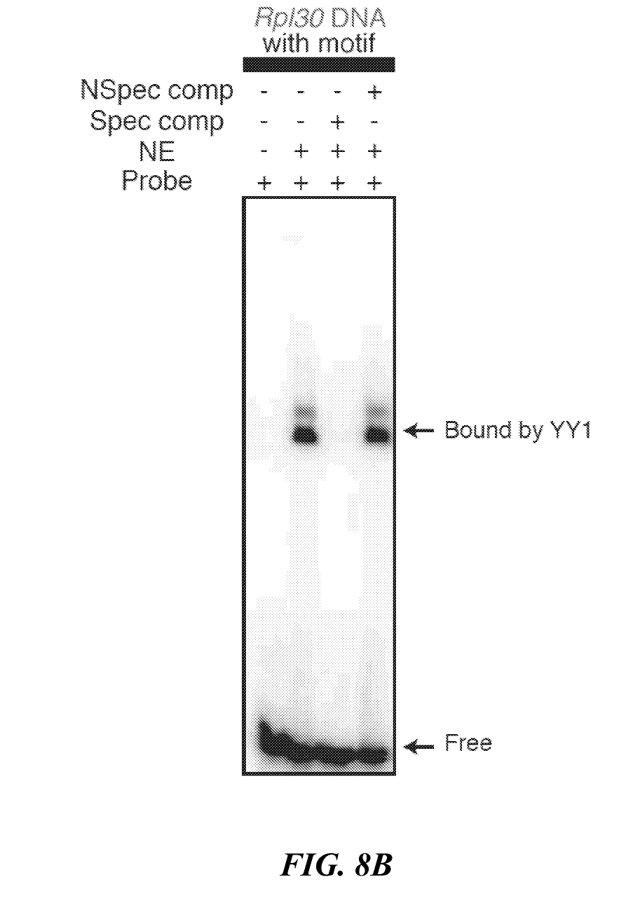

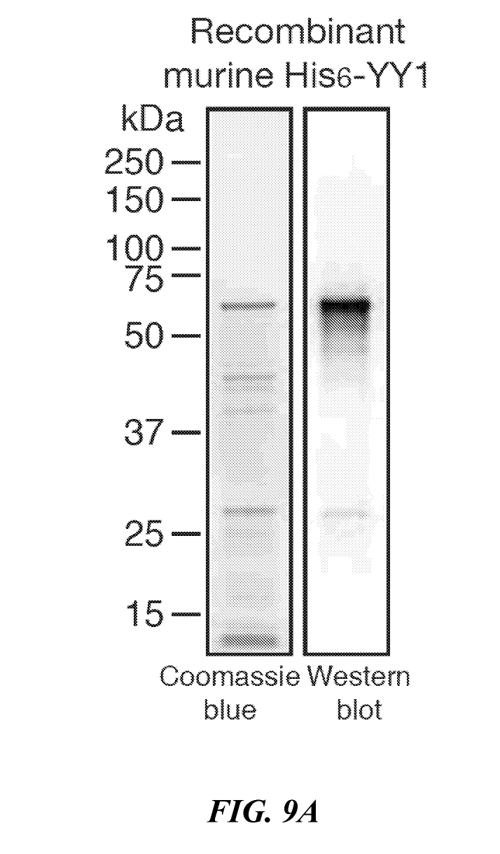

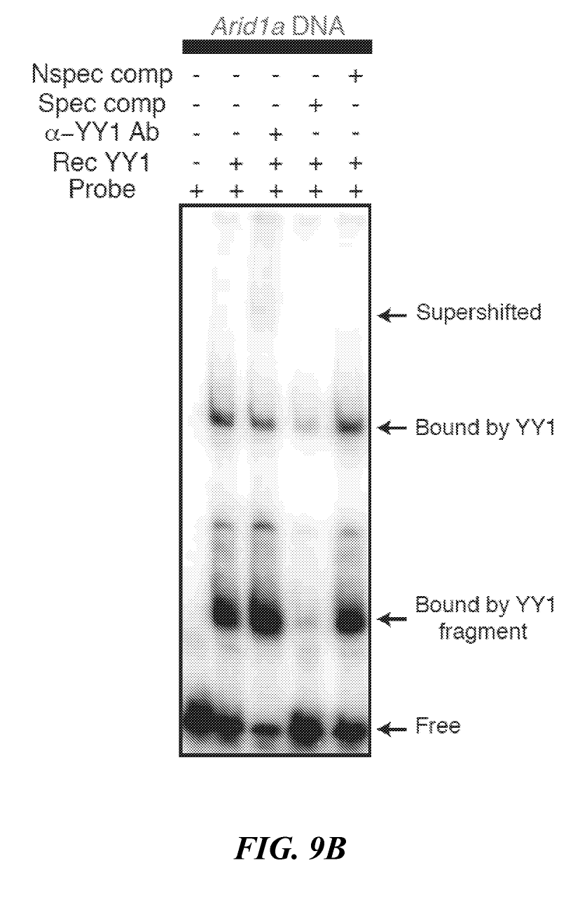

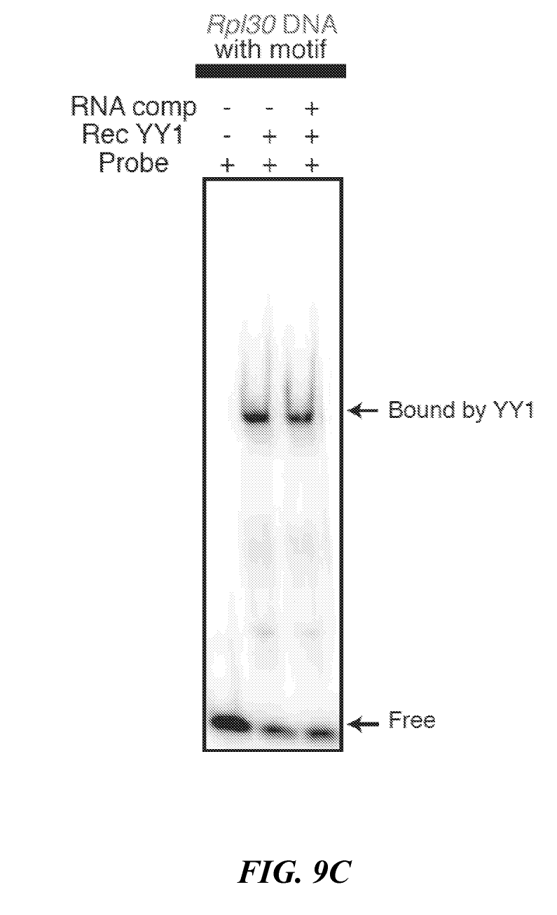

Based on A₂₆₀ to A₂₈₀ ratio, less than 4% of protein is associated with nucleic acid. HPLC chromatograph does not show presence of soluble YY1 aggregate. Recombinant YY1 behaves similarly to endogenous YY1 in nuclear extract EMSA assays as shown in FIG. 8 and FIG. 9.

Concentration of the full-length YY1 in the final protein preparation was determined by first resolving serial dilutions of BCA standard alongside YY1 on a 10% SDS-PAGE gel. Resolved proteins were then stained with the Bio-Safe Coomassie Stain (Bio-Rad), and YY1 amount was estimated by densitometry of the corresponding Coomassie-stained YY1 band relative to the BCA standards using ChemiDoc XRS+ system with Image Lab software (Bio-Rad).

The N-terminal (amino acids 1-277) and C-terminal (amino acids 271-420) portions of YY1 were purified in a fashion similar to the full-length YY1.

RNase Treatment and YY1 Chromatin Binding Assay:

Chromatin binding assay was modified from a published protocol (Cernilogar et al., 2011). Nuclei were prepared using hypotonic buffer and then half of the purified nuclei was left untreated (control), whereas another half was treated with 1:100 dilution of RNase A (Sigma, R4642) for 10 min at 37° C. After washing, both untreated and RNase A-treated nuclei were digested with 1:10 dilution of DNase I (Promega, M6101) in presence of (NH₄)₂SO₄ at 37° C. for 30 min to isolate soluble chromatin fraction. The supernatants (soluble chromatin fraction) were then analyzed by Western blotting.

Electrophoretic Mobility Shift Assay (EMSA):

EMSA was performed essentially as described in (Mullen et al., 2011). To prepare nuclear extracts (NE), bio-YY1 mESCs were depleted of MEFs. Cells were then washed twice in cold PBS, collected, and resuspended in 5 ml ice-cold hypotonic lysis buffer (20 mM HEPES, pH 7.4, 20% glycerol, 10 mM NaCl, 1.5 mM MgCl₂, 0.2 mM EDTA, 0.1% Triton X-100, 0.5 mM dithiothreitol, 1 mM phenylmethylsulfonyl fluoride) in presence of cOmplete proteinase inhibitor. After 10 min incubation on ice, nuclei were spun down, resuspended in 0.5 ml of nuclear extraction buffer (hypotonic lysis buffer plus 420 mM NaCl) and rotated for 1 hr at 4° C. Supernatants were then clarified by centrifugation, aliquoted, and stored at −80° C. Protein concentrations were determined using the BCA protein assay (Life Technologies).

Oligonucleotide DNA probes containing YY1 binding sites were generated by first annealing 30-nt single-stranded oligonucleotides to obtain 100 μM stock of double-stranded oligonucleotides, and by then labeling 10 pmol of this stock with T4 polynucleotide kinase (New England Biolabs) and [γ-32P]-ATP (Perkin Elmer). DNA-RNA chimeric oligonucleotides used in the tethering experiments were annealed and labeled in a similar way. The 30-nt single-stranded RNA probes were labeled in the same way as the DNA probes. Unincorporated [γ-32P]-ATP was removed using G-25 spin columns (Roche). Labeled stocks were further diluted to obtain 0.1 μM stocks of labeled nucleic acids, 1 μl of which was used in the binding reactions.

EMSA with NE were performed as follows: DNA-binding reactions (20 μl) containing 20 mM HEPES-KOH, pH 7.5, 105 mM NaCl, 1.5 mM MgCl₂, 0.2 mM EDTA, 0.02% Triton X-100, 5% glycerol, 0.5 mM dithiothreitol, 500 ng poly(dI-dC), and 10 μg nuclear extract were pre-incubated with or without specific competitor (100 fold excess) at room temperature for 20 min. Following pre-incubation, 0.1 pmol of radiolabeled DNA probe was added to the reaction mixtures and they were incubated for another 80 min. Each reaction mixture was then mixed with Triple Dye Loading Buffer (National Diagnostics), loaded onto a native 5% polyacrylamide gel (acrylamide:bis, 39:1, National Diagnostics) containing 0.5×TBE and 1% glycerol, and electrophoresed in 0.5×TBE at 300 V for 10 min at room temperature, followed by at 250 V for 75 min at 4° C. After electrophoresis, the gels were dried at 80° C. for 50 min and exposed to a phosphorimager screen (Fuji). For antibody super-shift assays, NEs were first incubated with radiolabeled DNA probe for 20 min before 0.5 μl of the YY1 antibody was added to the reaction mixtures and incubation continued for another 80 min.

EMSAs with the recombinant murine YY1 were performed in a similar way as those with the NE. DNA-binding reactions (20 μl) contained 10 mM HEPES-KOH pH 7.5, 12 mM Tris-HCl pH 7.4, 50 mM NaCl, 50 mM KCl, 5 mM MgCl₂, 0.1 mM ZnCl₂, 0.01% NP-40, 5% glycerol, 500 ng poly(dI-dC), 0.5 mM DTT, and recombinant murine YY1. RNA-binding reactions (20 μl) contained 10 mM HEPES-KOH pH 7.5, 12 mM Tris-HCl pH 7.4, 50 mM NaCl, 50 mM KCl, 5 mM MgCl₂, 0.1 mM ZnCl₂, 0.01% NP-40, 5% glycerol, 10 U SUPERase In RNase inhibitor (Life Technologies), 0.5 mM DTT, and recombinant murine YY1.

DNA Oligonucleotides Used as Probes and Competitors in EMSA:

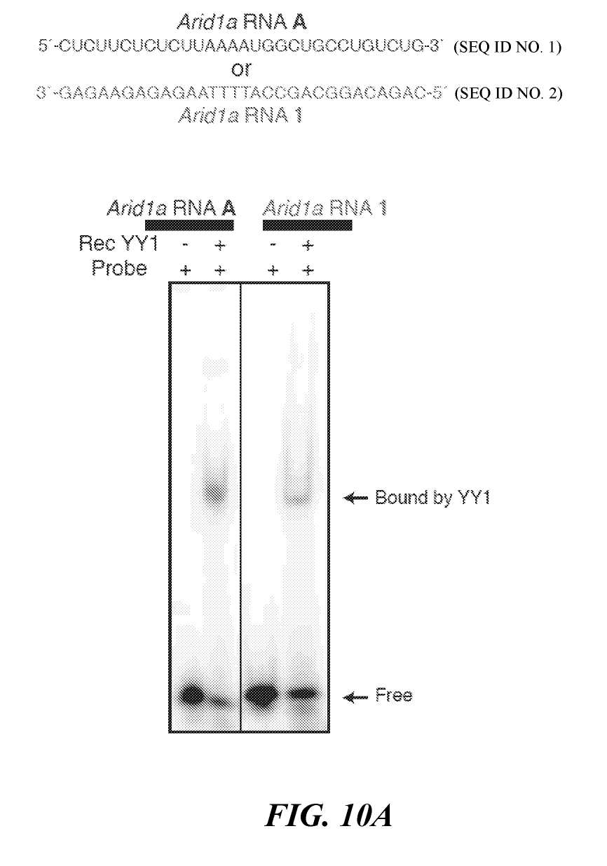

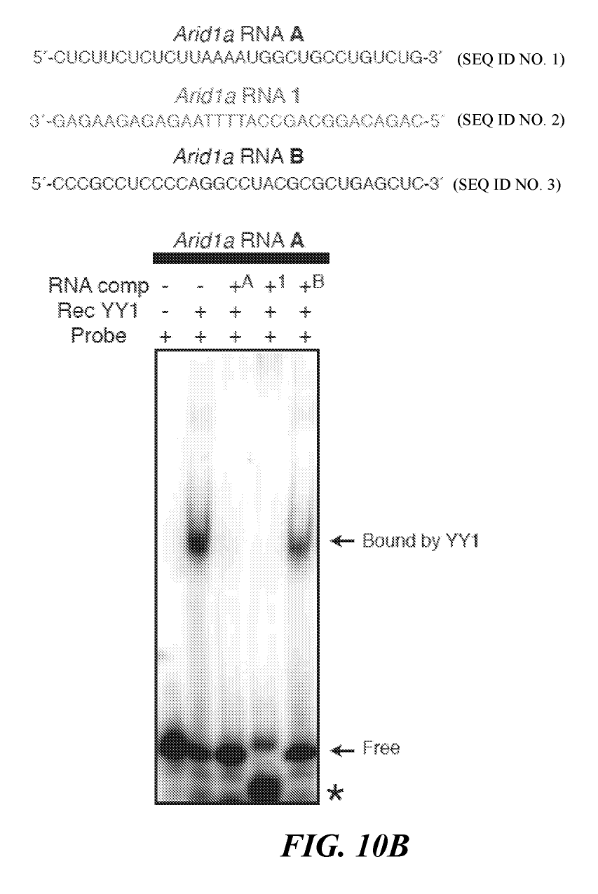

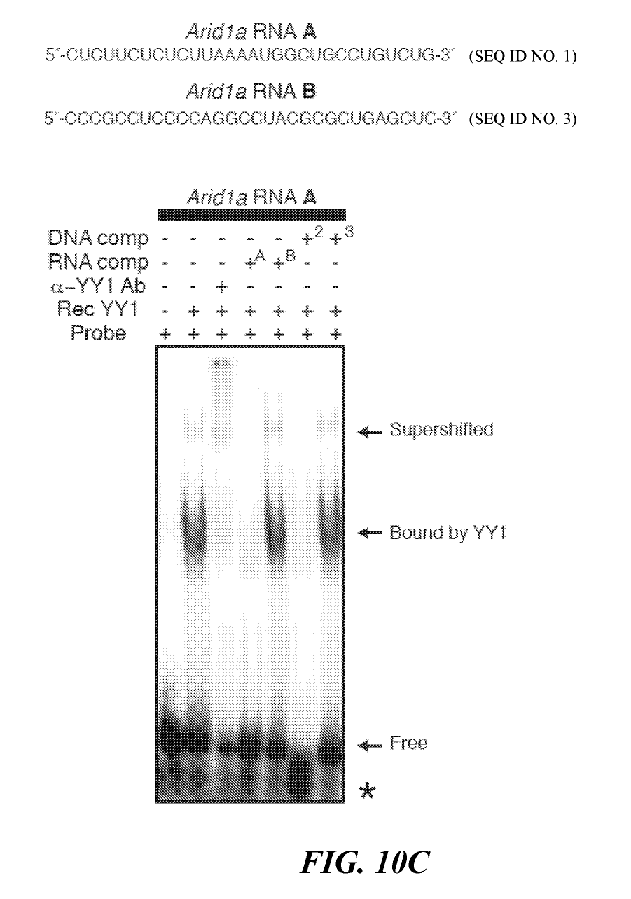

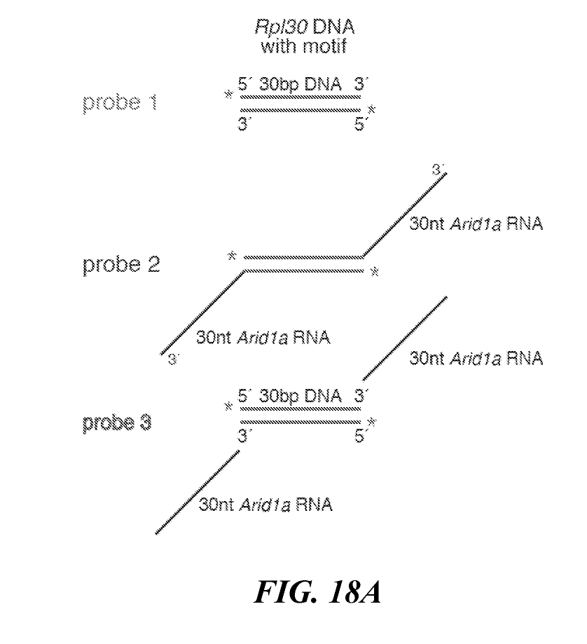

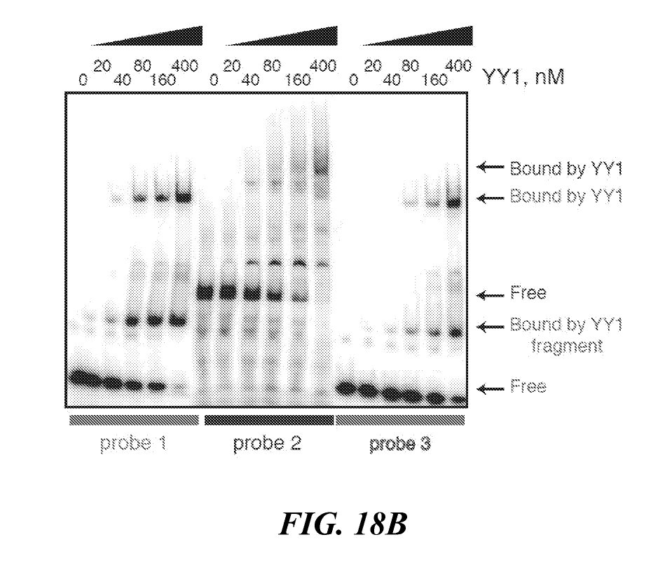

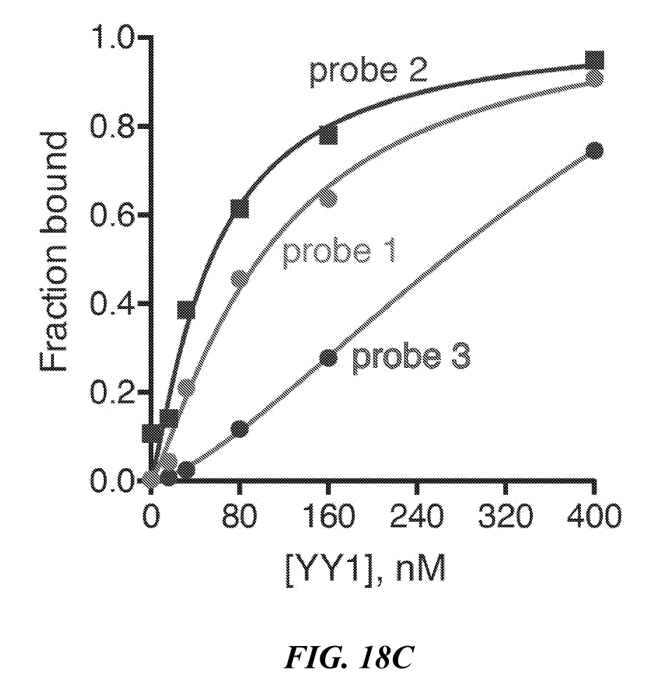

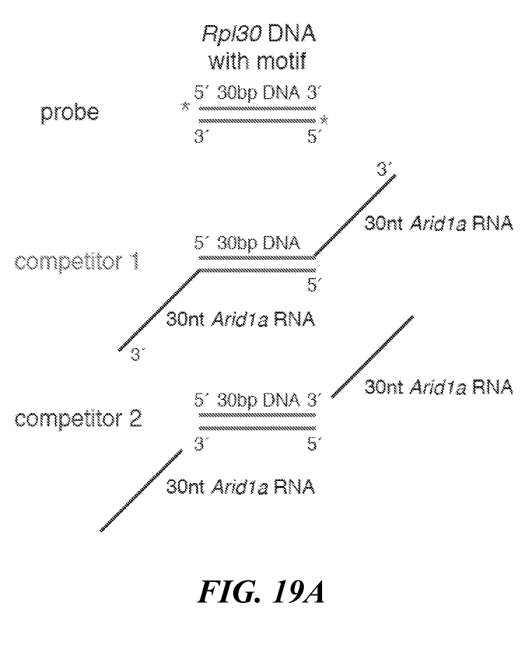

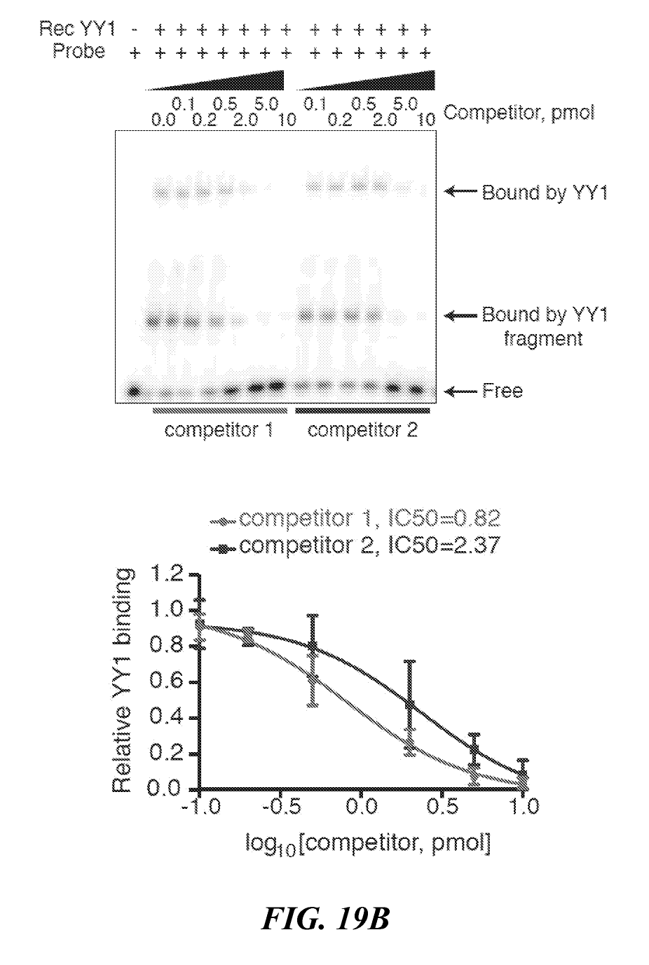

Rpl30 promoter with YY1 motif (labeled DNA probe in FIG. 8A, FIG. 8B, FIG. 9C, FIG. 18A, FIG. 18B, FIG. 18C, FIG. 19A, and FIG. 19B; cold specific competitor in FIG. 8B, cold DNA competitor 3 in FIG. 10C, and a part of cold competitor 2 in FIG. 19A and FIG. 19B):

Forward
(SEQ ID NO: 17)
5′-TCGCTCCCCGGCCATCTTGGCGGCTGGTGT-3′
Reverse
(SEQ ID NO: 18)
5′-ACACCAGCCGCCAAGATGGCCGGGGAGCGA-3

Rpl30 promoter with no YY1 motif (labeled probe in FIG. 8A and cold non-specific competitor in FIG. 8B):

Forward
(SEQ ID NO: 19)
5′-AGGCAGTGGCtAGcTcaCGTCCCTGGATCG-3′
Reverse
(SEQ ID NO: 20)
5′-CGATCCAGGGACGtgAgCTaGCCACTGCCT-3′

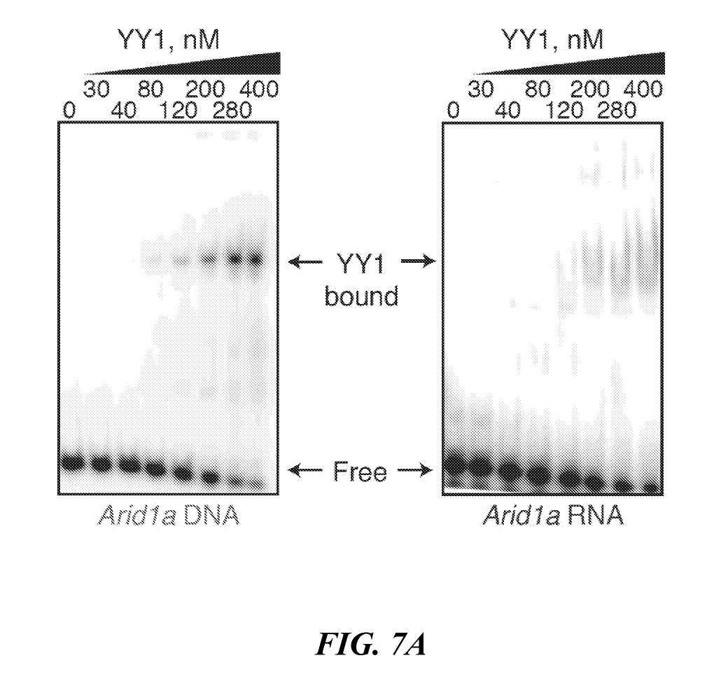

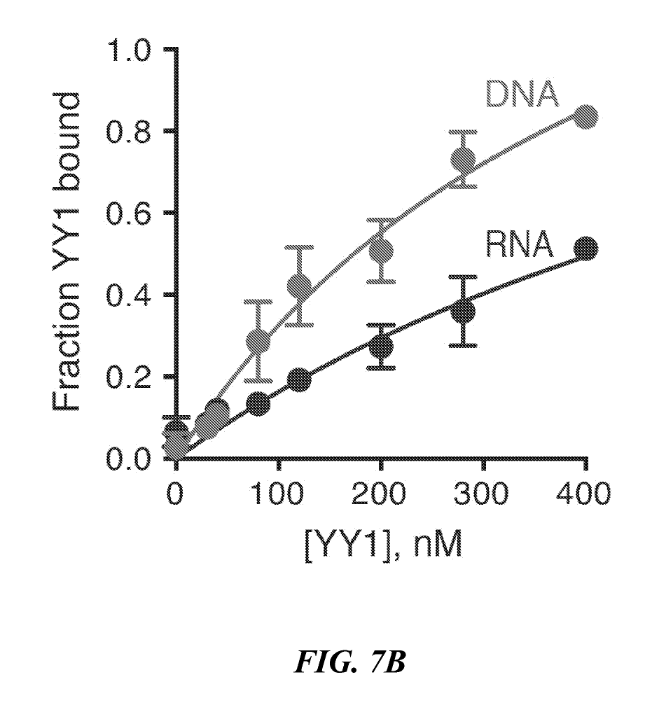

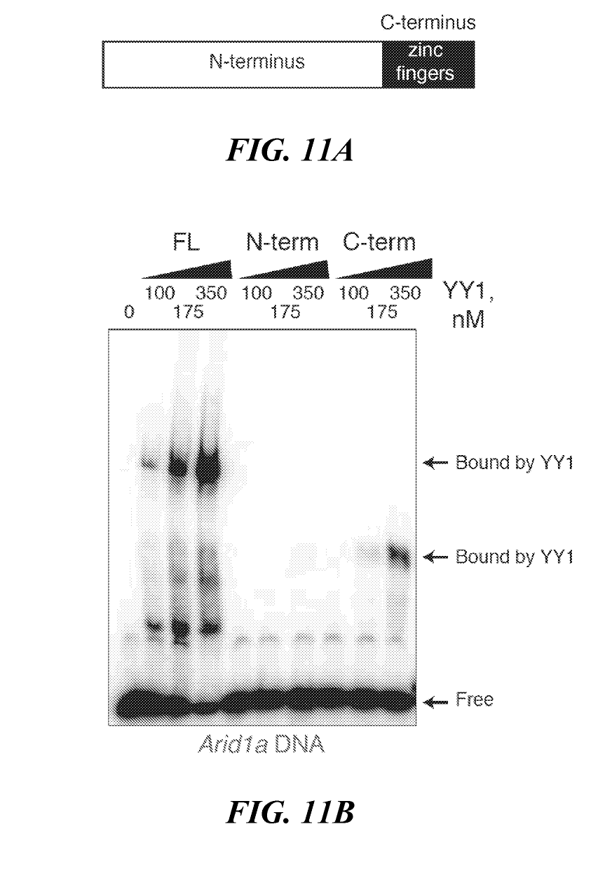

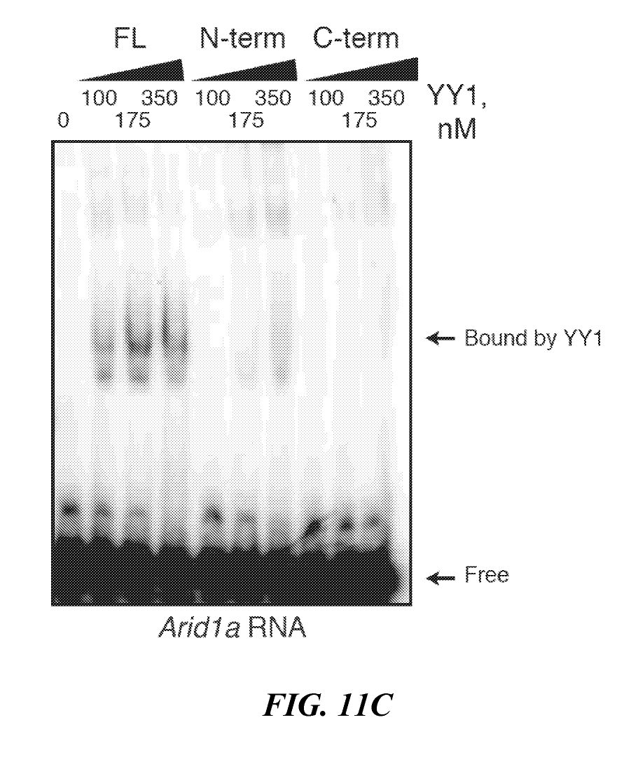

Arid1a promoter with YY1 motif (labeled DNA probe in FIG. 7A, FIG. 7B, FIG. 8A, FIG. 9B, and FIG. 11B; cold competitor in FIG. 9B and DNA competitor 2 in FIG. 9C):

Forward
(SEQ ID NO: 4)
5′-CTCTTCTCTCTTAAAATGGCTGCCTGTCTG-3′
Reverse
(SEQ ID NO: 21)
5′-CAGACAGGCAGCCATTTTAAGAGAGAAGAG-3′

Arid1a promoter with no YY1 motif (labeled DNA probe in FIG. 8A; cold non-specific competitor in FIG. 9B):

Forward
(SEQ ID NO: 22)
5′-CCCGCCTCCCCAGGCCTACGCGCTGAGCTC-3′
Reverse
(SEQ ID NO: 23)
5′-GAGCTCAGCGCGTAGGCCTGGGGAGGCGGG-3′

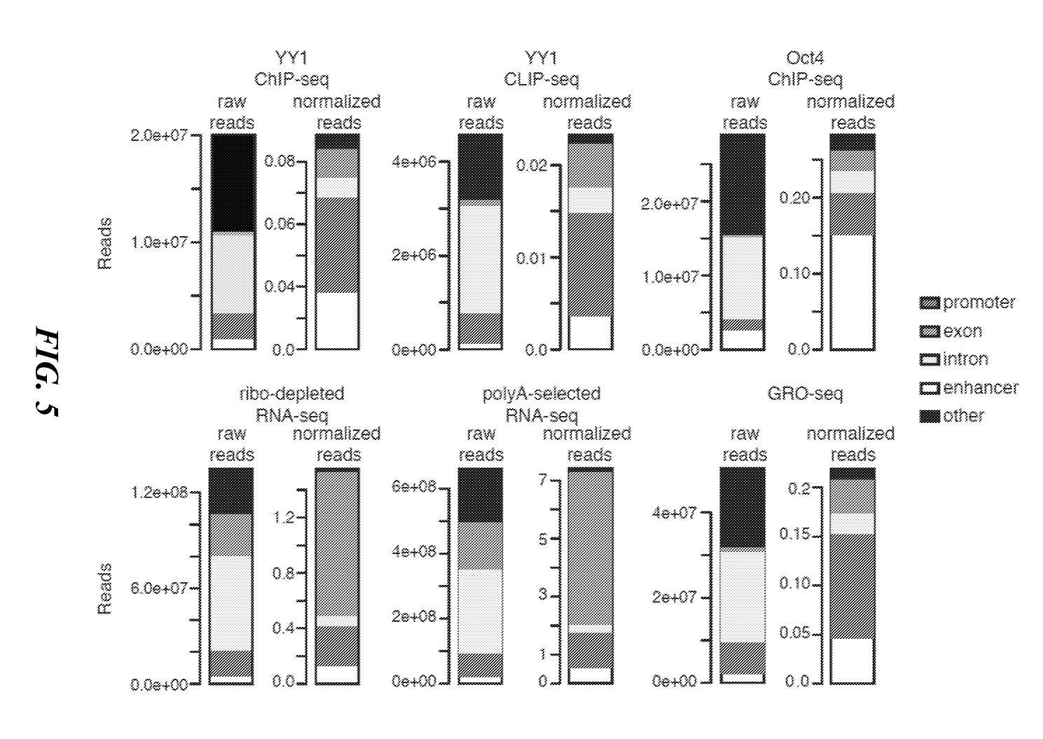

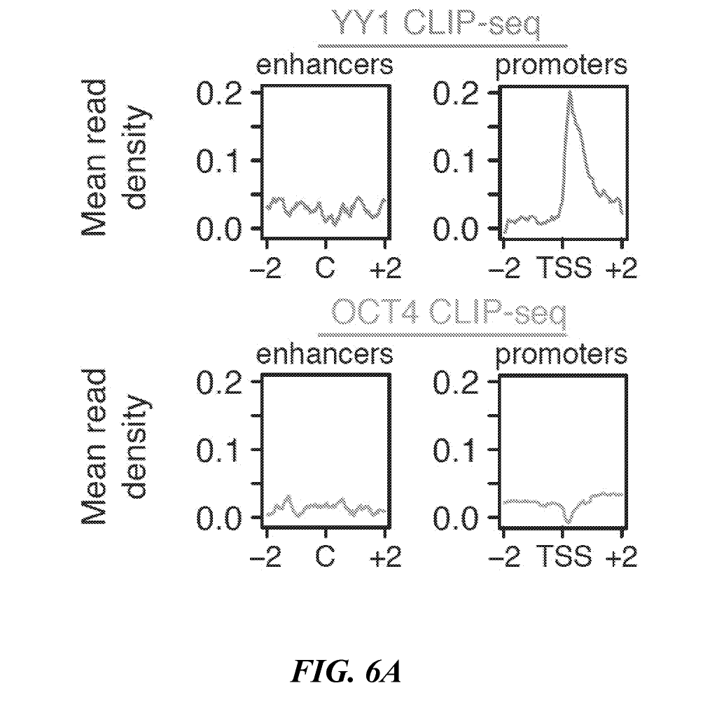

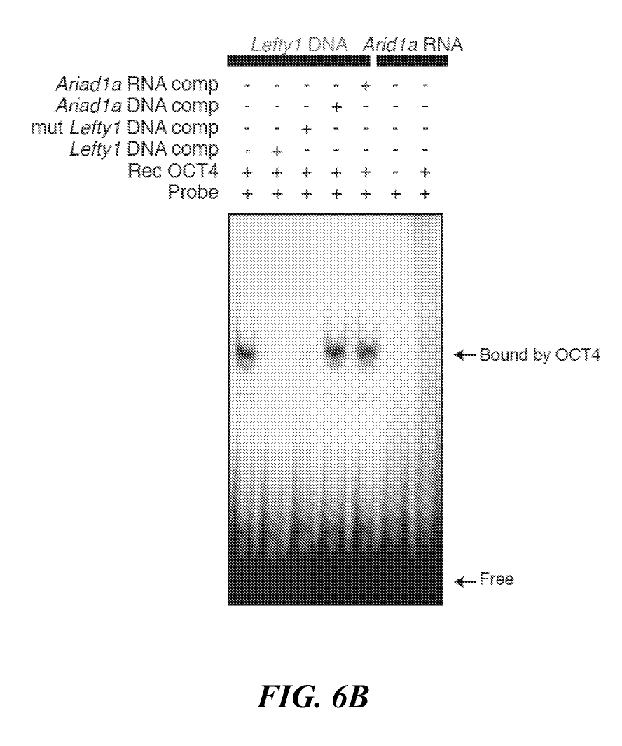

Lefty1 enhancer (labeled DNA probe in FIG. 6B and cold DNA competitor in FIG. 6B):

Forward
(SEQ ID NO: 24)
5′-GGTGGGAGGGAGACTGCCCTTTGTCATGTAGAAGGAGCTT-3′
Reverse
(SEQ ID NO: 25)
5′-AAGCTCCTTCTACATGACAAAGGGCAGTCTCCCTCCCACC-3′

mut Lefty1 enhancer (cold DNA competitor in FIG. 6B):

Forward
(SEQ ID NO: 26)
5′-ACGTGCAGGGAGACTGCCCTTTGTCATGTAGAAGCAGTTG-3′
Reverse
(SEQ ID NO: 27)
5′-CAACTGCTTCTACATGACAAAGGGCAGTCTCCCTGCACGT-3′

RNA Oligonucleotides Used as Probes and Competitors in EMSA:

Arid1a promoter RNA A (labeled probe in FIG. 7A and FIG. 7B, in FIG. 6B, FIG. 9A, FIG. 9B, FIG. 9C, FIG. 11C, and as a part of probe 2 in FIG. 18A, FIG. 18B, and FIG. 18C; cold competitor in FIG. 6B, FIG. 9C, FIG. 10B and FIG. 10C, a part of probe 3 in FIG. 18A, FIG. 18B, and FIG. 18C, and a part of cold competitor 1 and 2 in FIG. 19A and FIG. 19B):

(SEQ ID NO: 1)
5′-rCrUrCrUrUrCrUrCrUrCrUrUrArArArArUrGrGrCrUrGrCr
CrUrGrUrCrUrG-3′

Arid1a promoter RNA 1 (labeled probe in FIG. 10A; cold competitor in FIG. 10B):

(SEQ ID NO: 28)
5′-rCrArGrArCrArGrGrCrArGrCrCrArUrUrUrUrArArGrArGr
ArGrArArGrArG-3′

Arid1a promoter RNA B (cold competitor in FIG. 10B and FIG. 10C):

(SEQ ID NO: 3)
5′-rCrCrCrGrCrCrUrCrCrCrCrArGrGrCrCrUrArCrGrCrGrCr
UrGrArGrCrUrC-3′

DNA RNA Chimeric Oligonucleotides Used in In Vitro Tethering Experiments:

Rpl30 promoter with YY1 motif -Arid1a promoter RNA (labeled probe 2 in FIG. 18A, FIG. 18B, and FIG. 18C; cold competitor 1 in FIG. 19A and FIG. 19B):

Forward
(SEQ ID NO: 29)
5′-
TCGCTCCCCGGCCATCTTGGCGGCTGGTGTrCrUrCrUrUrCrUrCrUrC
rUrUrArArArArUrGrGrCrUrGrCrCrUrGrUrCrUrG-3′
(SEQ ID NO: 30)
ACACCAGCCGCCAAGATGGCCGGGGAGCGArCrUrCrUrUrCrUrCrUrC
rUrUrArArArArUrGrGrCrUrGrCrCrUrGrUrCrUrG-3′

Reverse 5′-

Competition EMSA: In Vitro RNA Tethering Experiments:

For competition EMSA in FIG. S15, single-stranded DNA-RNA chimeric oligonucleotides were annealed to obtain 50 μM stock of the 30-bp Rpl30 DNA containing 30-nt Arid1a RNA overhang on each 3′end (competitor 1) and its serial dilutions immediately before the experiment. Thus, each molecule of competitor 1 contained three potential binding sites for YY1: one in DNA, and two in RNA. Competitor 2 was obtained by first annealing complementary single-stranded Rpl30 DNA oligonucleotides to obtain 100 μM stock of 30-bp Rpl30 DNA, and by then mixing this stock with Arid1a promoter RNA to obtain a final stock, which consisted of 50 μM 30-bp Rpl30 DNA and 100 μM 30-nt Arid1a RNA. Thus, for each molecule of DNA, there were two molecules of RNA, which added up to three potential YY1 binding sites, as was the case in the competitor 1. The stock of the competitor 2 was further serially diluted before each experiment.

Binding reactions (20 μl) containing 10 mM HEPES-KOH pH 7.5, 12 mM Tris-HCl pH 7.4, 50 mM NaCl, 50 mM KCl, 5 mM MgCl₂, 0.1 mM ZnCl₂, 0.01% NP-40, 5% glycerol, 10 U SUPERase In RNase inhibitor (Life Technologies), 0.5 mM DTT, 500 ng poly(dI-dC), and recombinant murine YY1 were pre-incubated with or without different concentrations of either competitor 1 or competitor 2 at room temperature for 20 min. Following pre-incubation, 0.1 pmol of radiolabeled DNA probe was added to the reaction mixtures and binding reactions were incubated for another 80 min. Each reaction mixture was then mixed with Triple Dye Loading Buffer (National Diagnostics) and loaded onto a native 5% polyacrylamide gel (acrylamide:bis, 39:1, National Diagnostics) containing 0.5×TBE and 1% glycerol and electrophoresed in 0.5×TBE at 300 V for 10 min at room temperature, followed by electrophoresis at 250 V for 75 min at 4° C.

After electrophoresis, the gels were dried at 80° C. for 50 min and exposed to a phosphorimager screen (Fuji). Screen were scanned using Typhoon FLA 9500 (GE Healthcare Life Sciences) and quantified using ImageQuant TL software (GE Healthcare Life Sciences). Graph in FIG. 19B was created and IC₅₀ values were estimated using Prism 6 software. Error bars represent standard deviation from the mean value for each data point obtained in at least three replicates. Values were fit using non-linear regression function in the Prism program. The p-value for differences between log 10IC₅₀ values was calculated using the two-tailed t-test taking into account number of biological replicates for each data point.

Generation of Stable mESC Lines for In Vivo Tethering Experiments:

Genome editing was performed using CRISPR/Cas9 essentially as described (Wang et al., 2013; Kearns et al., 2014). Lentiviral plasmid pHAGE-TRE-dCas9 containing tetracycline-inducible dCas9 and lentiviral stuffer plasmid pLKO.1-puro U6 sgRNA BfuAI were obtained from Addgene (50915 and 50920, respectively).

Target-specific DNA oligonucleotides were annealed and cloned into the pLKO.1-puro U6 sgRNA BfuAI plasmid digested with BfuAI to obtain pLKO.1-puro U6 sgSuz12, pLKO.1-puro U6 sgKlf5, pLKO.1-puro U6 sgE2f3, pLKO.1-puro U6 sgNufip2, pLKO.1-puro U6 sgCnot6, and pLKO.1-puro U6 sgPias1 plasmids. The genomic sequences complementary to guide RNAs are GTACTGGCTGCTCAAATGTC (SEQ ID NO: 31) for Suz12 enhancer, GTGATGTAGGTATAATTAGCC (SEQ ID NO: 32) for Klf5 enhancer, TTGCATGTTGTTCCTCGGAGC (SEQ ID NO: 33) for E2f3 enhancer, CCATGTATGGTTACGGGGATC (SEQ ID NO: 34) for Nufip2 enhancer, GCTGAAAGACCACAGCTCCC (SEQ ID NO: 35) for Cnot6 enhancer, and TTGGGTGTTGAGAATAGGTCC (SEQ ID NO: 36) for Pias1 enhancer. Constructs for targeting the RNA tethering constructs to the six enhancers were ordered from Integrated DNA Technologies as gBlocks and cloned into the pLKO.1-puro U6 sgRNA BfuAI plasmid digested with NdeI and EcoRI to obtain pLKO.1-puro U6 sgSuz12-tracrRNA-Arid1a RNA A and pLKO.1-puro U6 sgSuz12-tracrRNA-Arid1a RNA B constructs, pLKO.1-puro U6 sgCnot6-tracrRNA-Arid1a RNA A and pLKO.1-puro U6 sgCnot6-tracrRNA-Arid1a RNA B constructs, pLKO.1-puro U6 sgE2f3-tracrRNA-Arid/a RNA A and pLKO.1-puro U6 sgE2f3-tracrRNA-Arid/a RNA B constructs, pLKO.1-puro U6 sgKlf5-tracrRNA-Arid1a RNA A construct, pLKO.1-puro U6 sgNufip2-tracrRNA-Arid1a RNA A, and pLKO.1-puro U6 sgPias1-tracrRNA-Arid1a RNA A, in which Arid1a RNA A is the RNA compatible with YY1 binding in vitro, and Arid1a RNA B is the RNA incompatible with YY1 binding.

Puromycin-resistance (pac) gene in all targeting plasmids was then replaced with hygromycin B-resistance gene (HygR) by first amplifying HygR gene from pLVX-Tet-On Advanced vector (Clontech) using Phusion High-Fidelity DNA polymerase (NEB), digesting it with BamHI and KpnI restriction enzymes, and then cloning the digested PCR product into the targeting plasmids digested with the same restriction enzymes to obtain pLKO.1-hygro U6 sgSuz12-tracrRNA-Arid1a RNA A and pLKO.1-hygro U6 sgSuz12-tracrRNA-Arid1a RNA B constructs, pLKO.1-hygro U6 sgCnot6-tracrRNA-Arid1a RNA A and pLKO.1-hygro U6 sgCnot6-tracrRNA-Arid1a RNA B constructs, pLKO.1-hygro U6 sgE2f3-tracrRNA-Arid/a RNA A and pLKO.1-hygro U6 sgE2f3-tracrRNA-Arid/a RNA B constructs, pLKO.1-hygro U6 sgKlf5-tracrRNA-Arid/a RNA A construct, pLKO.1-hygro U6 sgNufip2-tracrRNA-Arid1a RNA A, and pLKO.1-hygro U6 sgPias1-tracrRNA-Arid1a RNA A plasmids.

pHAGE-TRE-dCas9 DNA and each of the targeting plasmids were packaged into lentivirus in the pairwise fashion by transfecting the corresponding plasmids into the HEK 293FT cells (Life Technologies) using Lipofectamine 2000 transfection reagent (Life Technologies) in the presence of packaging plasmids.

mESCs expressing bio-YY1 (Vella et al., 2012) were depleted of MEFs and transduced with the lentivirus for the expression of dCas9 and each of the targeting RNA constructs. Selection for cells expressing both dCas9 and each of the targeting constructs was conducted in presence of 500 μg/ml of gentamycin and 500 μg/ml of hygromycin B. Selection began 24 hrs from the start of transduction and continued for another 7 days. Once selection was finished, cells were maintained on DR4 MEFs in media containing 100 μg/ml of gentamycin and 100 μg/ml of hygromycin B. Expression of dCas9 was induced with 1 μg/ml of doxycycline and cells were collected 72 hrs later. Cells were crosslinked using formaldehyde in three biological replicates and the extent of YY1 binding at targeted and control untargeted enhancers in the same cells was evaluated after affinity purification of YY1-associated DNA followed by qPCR.

Affinity Purification and qPCR Analysis (ChIP-qPCR) of YY1-Associated DNA in the In Vivo Tethering Experiments:

Approximately 2×10⁷ mESCs was chemically crosslinked by the addition of one-tenth volume of fresh 11% formaldehyde solution for 10 minutes at room temperature. Formaldehyde was quenched by addition of one-twentieth volume of 2.5M Glycine and incubation for another 5 min at room temperature. Cells were rinsed twice with 25 ml of ice-cold 1× PBS and harvested using a silicon scraper, pelleted down by centrifugation at 1,500 g for 5 min at 4° C., and pellets were flash-frozen in liquid nitrogen. Cells were stored at −80° C. prior to use.

Cells were lysed in 4 ml of LB1 (50 mM HEPES-KOH pH 7.5, 140 mM NaCl, 1 mM EDTA, 0.25% Triton X-100, 0.5% NP-40, 10% glycerol) in presence of cOmplete proteinase inhibitors (Roche) and incubated on a rotator at 4° C. for 10 min. After centrifugation at 1350 g for 5 min at 4° C., nuclei were washed with 4 ml of LB2 (10 mM Tris-HCl pH 8.0, 200 mM NaCl, 1 mM EDTA pH 8.0, 1 mM EGTA pH 8.0 in presence of cOmplete proteinase inhibitors (Roche)) by incubating them on a rotator at 4° C. for 10 min. After centrifugation at 1350 g for 5 min at 4° C., nuclei were resuspended in 2 ml of the final sonication buffer (50 mM HEPES-KOH pH 7.5, 140 mM NaCl, 1 mM EDTA, 1% Triton X-100, 0.1% SDS, 0.1% sodium deoxycholate in presence of cOmplete proteinase inhibitors (Roche)) and sonicated on ice (7 cycles at 30 seconds each at 18 watts 60 second pause between pulses, output level 4.5) using Misonix Sonicator 3000. The resulting whole cell extract was cleared by centrifugation for 10 min at 20,000 g and then incubated overnight at 4° C. with 50 μl Dynabeads® MyOne™ Streptavidin T1 (Life Technologies, 65601) magnetic beads pre-blocked with 0.5% BSA in PBS for 2 hrs at 4° C. Beads were washed at room temperature for 8 min with each of the following buffers: 2× with 2% (vol/vol) SDS in PBS; 1× with 50 mM HEPES pH 7.5, 350 mM NaCl, 1 mM EDTA, 1% Triton X-100, 0.1% sodium deoxycholate, 0.1% SDS; 1× with 20 mM Tris-HCl pH 8.0, 250 mM LiCl, 1 mM EDTA, 0.5% NP40, 0.5% sodium deoxycholate; 2× with TE.

DNA was eluted off beads in 300 μl of elution buffer (50 mM Tris-HCl pH 8.0, 10 mM EDTA pH 8.0, 1% SDS) by rotating the tubes containing the beads overnight at 65° C. SDS in the eluted sample was diluted two-fold with TE buffer, and RNA was digested by incubating the tubes at 37° C. for 30 min in presence of 3 μl of RNase A (30 mg/ml). Following incubation, proteins were digested with 7 μl of 20 mg/ml Proteinase Kin presence of 3 μl of 1M CaCl₂ at 55° C. for 1.5 hrs. After phenol/chloroform extraction, DNA was ethanol precipitated and resuspended in 50 μl.

DNA isolated from 5% of the initial whole-cell extract (input DNA) was similarly resuspended in 50 μl of H₂O.

1 μl of the affinity-purified YY1-associated DNA or input DNA was used in qPCR reactions containing 1× Power SYBR Green PCR master mix (Life Technologies). Arid1a enhancer, bound by YY1 in mESCs, was chosen as a positive control region, whereas olfactory receptor gene, which has no YY1 binding in mESCs, was chosen as a negative control region. First, fold enrichment in YY1 binding at Arid1a enhancer in each of the ChIP samples over the YY1 binding at the same enhancer in the corresponding input samples was estimated relative to the fold enrichment at the negative control region in the same samples as a measure of the affinity purification efficiency among different samples. As a result, to compensate for differences in affinity purification efficiency among different samples, normalization coefficients were derived for each ChIP sample. Then, fold enrichment in YY1 binding at each of the six targeted enhancers and at three not targeted enhancers in each of the ChIP samples over the corresponding input samples was estimated relative to the fold enrichment at the negative control region in the same samples. These fold enrichment values were then multiplied by the normalization coefficients to allow comparison between different samples. Finally, fold enrichment at the targeted enhancers in cells containing tethered RNA was compared to fold enrichment at these enhancers in cells containing only the corresponding guide RNA (sgRNA). The differences in the fold enrichments were expressed as fold change in YY1 binding in cells containing tethered RNA relative to cells containing only the corresponding guide RNA.

Primer sequences were the following:

Arid1a enhancer
Forward
(SEQ ID NO: 37)
GGCAAACTTTCGGTTCAGTGG
Reverse
(SEQ ID NO: 38)
TTTTCCTCCCCCAAGACAGG
Negative control region
Forward
(SEQ ID NO: 39)
CCCACCTTGTGTTCAAATGCTGA
Reverse
(SEQ ID NO: 40)
ACGCTTTTCTTCTGCCTTCTGC
Cnot6 enhancer
Forward
(SEQ ID NO: 41)
AACCATCAGGAAGGCATCAGG
Reverse
(SEQ ID NO: 42)
AGCTGATAGGCACTCTGGGTA
Suz12 enhancer
Forward
(SEQ ID NO: 43)
GATCTCCGTACAAGCAGGAGG
Reverse
(SEQ ID NO: 44)
ACTCACCTAAATCTGCCTGCC
E2f3 enhancer
Forward
(SEQ ID NO: 45)
AGATGCCAGAGGTCACCTTTG
Reverse
(SEQ ID NO: 46)
CAAGAGGATTTGTGGGGCTCT
Nufip2 enhancer
Forward
(SEQ ID NO: 47)
TACACGTGTGCGCCAGAAGAG
Reverse
(SEQ ID NO: 48)
TCTAGGAGCCCAGCCCCTTTT
Klf5 enhancer
Forward
(SEQ ID NO: 49)
CTCCATTGTCCTAGGGATGCC
Reverse
(SEQ ID NO: 50)
ACTTTATTCACGGGGCTCCAG
Pias1 enhancer
Forward
(SEQ ID NO: 51)
ACTGACTTCCTCCAAGGCCAC
Reverse
(SEQ ID NO: 52)
TGAATGCTTGGTCCCCAGTGT

p-values for the in vivo tethering experiments are shown below:

TABLE 3
Fold change in YY1 binding relative to control

| | | | | p-value for |
|---|---|---|---|---|
| | | | | changes relative |
| | exp1 | exp2 | exp3 | to control |
| Targeted | 1.64 | 1.36 | 1.54 | 0.01 |
| enhancer Suz12 | | | | |
| not-targeted | 0.75 | 0.85 | 0.91 | 0.96 |
| enhancer Cnot6 | | | | |
| not targeted | 1.19 | 1.03 | 1.16 | 0.06 |
| enhancer E2f3 | | | | |
| not targeted | 1.22 | 0.80 | 1.13 | 0.37 |
| enhancer Klf5 | | | | |
| Targeted | 1.14 | 1.34 | 1.32 | 0.03 |
| enhancer Cnot6 | | | | |
| not targeted | 0.87 | 0.82 | 1.18 | 0.63 |
| enhancer Suz12 | | | | |
| not targeted | 0.81 | 0.72 | 0.91 | 0.96 |
| enhancer E2f3 | | | | |
| not targeted | 1.06 | 1.12 | 0.89 | 0.38 |
| enhancer Klf5 | | | | |
| Targeted | 1.5 | 1.58 | 1.43 | 0.00 |
| enhancer E2f3 | | | | |
| not targeted | 1.30 | 1.22 | 1.31 | 0.01 |
| enhancer Cnot6 | | | | |
| not targeted | 1.08 | 1.15 | 1.29 | 0.05 |
| enhancer Suz12 | | | | |
| not targeted | 0.74 | 1.01 | 0.75 | 0.90 |
| enhancer Klf5 | | | | |
| Targeted | 1.64 | 1.81 | 2.41 | 0.03 |
| enhancer Klf5 | | | | |
| not targeted | 0.96 | 0.69 | 0.87 | 0.91 |
| enhancer Suz12 | | | | |
| not targeted | 1.02 | 0.68 | 1.10 | 0.67 |
| enhancer E2f3 | | | | |
| not targeted | 0.89 | 0.86 | 0.83 | 0.99 |
| enhancer Cnot6 | | | | |
| Targeted | 1.51 | 1.39 | 1.31 | 0.01 |
| enhancer Pias1 | | | | |
| not targeted | 0.79 | 1.09 | 1.05 | 0.59 |
| enhancer Suz12 | | | | |
| not targeted | 1.10 | 1.12 | 0.82 | 0.45 |
| enhancer E2f3 | | | | |
| not targeted | 1.19 | 0.98 | 1.15 | 0.12 |
| enhancer Cnot6 | | | | |
| Targeted | 1.87 | 2.18 | 1.42 | 0.03 |
| enhancer | | | | |
| Nufip2 | | | | |
| not targeted | 0.85 | 0.97 | 1.19 | 0.49 |
| enhancer Suz12 | | | | |
| not targeted | 1.05 | 1.07 | 1.02 | 0.04 |
| enhancer E2f3 | | | | |
| not targeted | 0.82 | 0.92 | 0.76 | 0.96 |
| enhancer Cnot6 | | | | |

TABLE 4
GEO numbers for the datasets used in this study.

| | | |
|---|---|---|
| Experiment | IP Sample ID | Control Sample ID |

FIG. 1B metagenes

| | | |
|---|---|---|
| GRO-Seq | GSM1665566 | N/A |

FIG. 1C Arid1a track

| | | |
|---|---|---|
| OSN merged ChIP-Seq | GSM1082340, | N/A |
| | GSM1082341, | |
| | GSM1082342 | |
| YY1 ChIP-Seq | GSM1665561 | N/A |
| YY1 CLIP-Seq | GSM1665564 | N/A |
| Control CLIP-Seq | GSM 1665565 | N/A |
| GRO-Seq | GSM 1665566 | N/A |

FIG. 1D metagenes

| | | |
|---|---|---|
| Oct4 ChIP-Seq | GSM1082340 | N/A |
| YY1 ChIP-Seq | GSM1665561 | N/A |
| YY1 CLIP-Seq | GSM1665564 | N/A |

FIG. 1E heatmaps

| | | |
|---|---|---|
| YY1 ChIP-Seq | GSM1665561 | N/A |
| YY1 CLIP-Seq | GSM1665564 | N/A |

FIG. 12B metagenes

| | | |
|---|---|---|
| Untreated GRO-Seq | GSM1579223 | N/A |
| DRB GRO-Seq | GSM1579224 | N/A |
| Untreated YY1 ChIP-Seq | GSM1665555 | GSM1665554, |
| | | GSM1665557, |
| | | GSM1665560 |
| DRB YY1 ChIP-Seq | GSM1665556 | GSM1665554, |
| | | GSM1665557, |
| | | GSM1665560 |

FIG. 12C metagenes

| | | |
|---|---|---|
| DRB −> Release GRO-Seq | GSM1579227 | N/A |
| DRB GRO-Seq | GSM1579224 | N/A |
| DRB −> Release YY1 ChIP- | GSE68195 | GSM1665554, |
| Seq | | GSM1665557, |
| | | GSM1665560 |
| DRB YY1 ChIP-Seq | GSM1665556 | GSM1665554, |
| | | GSM1665557, |
| | | GSM1665560 |

FIG. 12D boxplots/metagenes

| | | |
|---|---|---|
| RNA-Seq | GSE68198 | GSE68195 |
| shLuc YY1 ChIP-Seq | GSE68195 | GSE68195 |
| shExo10 YY1 ChIP-Seq | GSE68195 | GSE68195 |

FIG. 2

| | | |
|---|---|---|
| GRO-Seq | GSM1665566 | N/A |
| OCT4 ChIP-Seq | GSM1082340 | N/A |
| YY1 ChIP-Seq | GSM1665561 | N/A |
| YY1 CLIP-Seq | GSM1665564 | N/A |

FIG. 5

| | | |
|---|---|---|
| YY1 CLIP-Seq | GSM1665564 | N/A |
| YY1 ChIP-Seq | GSM1665561 | N/A |

FIG. 6

| | | |
|---|---|---|
| YY1 CLIP | GSM1665564 | GSM1665565 |
| Oct4 CLIP | GSE68196 | GSE68196 |

FIG. 11B

| | | |
|---|---|---|
| GRO-Seq | GSM1665567 | |
| GRO-Seq | GSM1665568 | |
| YY1 ChIP-Seq | GSM1665558 | GSM1665554, |
| | | GSM1665557, |
| | | GSM1665560 |
| ActD YY1 ChIP-Seq | GSM1665559 | GSM1665554, |
| | | GSM1665557, |
| | | GSM1665560 |

FIG. 11C

| | | |
|---|---|---|
| YY1 ChIP-Seq | GSM1665561 | GSM1665554, |
| | | GSM1665557, |
| | | GSM1665560 |
| THZ1 YY1 ChIP-Seq | GSM1665562 | GSM1665554, |
| | | GSM1665557, |
| | | GSM1665560 |
| TPI YY1 ChIP-Seq | GSM1665563 | GSM1665554, |
| | | GSM1665557, |
| | | GSM1665560 |

### Results

Active promoters and enhancer elements are transcribed bi-directionally (FIG. 1A) (Core et al., 2008; Seila et al., 2008; Sigova et al., 2013). Although various models have been proposed for the roles of RNA species produced from these regulatory elements, their functions are not fully understood (Kim et al., 2010; Wang et al., 2011; Melo et al., 2013; Lai et al., 2013; Lam et al., 2013; Li et al., 2013; Kaikkonen et al., 2013; Mousavi et al., 2013; Ruscio et al., 2013; Schaukowitch et al., 2014). Evidence that some DNA-binding transcription factors (TFs) also bind RNA (Cassiday et al., 2002; Jeon et al., 2011) led to the possibility that there might be a direct and general role for promoter-proximal and distal enhancer RNA in the binding and maintenance of TFs at regulatory elements.

Nascent transcripts (GRO-seq) in murine embryonic stem cells (ESCs) were sequenced at great depth, which confirmed that active promoters and enhancer elements are generally transcribed bi-directionally (FIG. 1B, FIG. 2A, Table 4). Studies were then focused on the TF Yin-Yang 1 (YY1) because it is ubiquitously expressed in mammalian cells, plays key roles in normal development, and can bind RNA species in vitro (Jeon et al., 2011; Gordon et al., 2006). ChIP-seq analysis in ESCs revealed that YY1 binds to both active enhancers and promoters, with some preference for promoters (FIG. 1C, FIG. 1D, FIG. 2, Table 5 in Appendix, which is disclosed in U.S. Provisional Application No. 62/248,119, filed Oct. 29, 2015, which is incorporated herein by reference in its entirety.

In contrast, the pluripotency TF OCT4 preferentially occupies enhancers (FIG. 2B). Consistent with this, YY1 sequence motifs were enriched at promoters, whereas OCT4 motifs were enriched at enhancers (FIG. 2B). Neither YY1 nor OCT4 occupied the promoter-proximal sequences of inactive genes (FIG. 3). These results establish that YY1 generally occupies active enhancer and promoter-proximal elements in ESCs.

YY1 binding to RNA was next investigated in vivo by using CLIP-seq in ESCs (FIG. 4, FIG. 5, Table 5 in Appendix, which is disclosed in U.S. Provisional Application No. 62/248,119, filed Oct. 29, 2015, which is incorporated herein by reference in its entirety. The results showed that YY1 binds RNA species at the active enhancer and promoter regions where it is bound to DNA (FIG. 1C, FIG. 1D, FIG. 2C). At promoters, YY1 preferentially occupied RNA downstream rather than upstream of transcription start sites (FIG. 2B), consistent with YY1 motif distribution and evidence that upstream ncRNA is unstable (Sigova et al., 2013; Flynn et al., 2011; Preker et al., 2008). In similar experiments with OCT4, significant levels of RNA binding were not observed (FIG. 6). These results suggest that YY1 generally binds to RNA species transcribed from enhancers and promoters in vivo.

The DNA and RNA binding properties of YY1 were further investigated in vitro (FIG. 7, FIG. 8; FIG. 9; FIG. 10). Recombinant murine YY1 protein bound both DNA and RNA probes in electrophoretic mobility shift essays (EMSA), showing higher affinity for DNA than RNA. There was variation in the affinity of YY1 for different RNA sequences (FIG. 10). The four YY1 zinc-fingers can bind DNA (Houbaviy et al., 1996), but the portion of YY1 that interacts with RNA is unknown. The zinc-finger—containing C-terminal region and the N-terminal region of YY1 were purified and their DNA and RNA binding properties were further investigated (FIG. 11). The zinc-finger region of YY1 bound to DNA, but not to RNA, whereas the N-terminal region of YY1 bound to RNA (FIG. 11). Furthermore, the DNA probe did not compete efficiently with the RNA probe for YY1 binding (FIG. 9C, FIG. 10C). These results suggest that different regions of YY1 are responsible for binding to DNA and RNA.

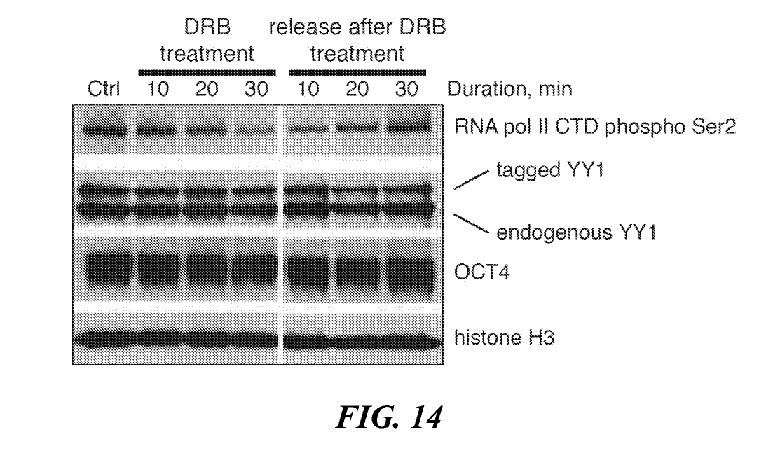

The observation that YY1 binds to enhancer and promoter-proximal elements and to RNA transcribed from those regions led us to postulate that nascent RNA contributes to stable TF occupancy at these regulatory elements (FIG. 12A). If this model is correct, then reduced levels of nascent RNA at promoters and enhancers might lead to reduced YY1 occupancy at these sites. Transcription elongation was briefly inhibited with the reversible inhibitor D-rybofuranosylbenzimidazole (DRB) to reduce RNA levels at promoters and enhancers without causing changes in the steady-state levels of YY1 (FIG. 13, FIG. 14). DRB treatment reduced transcription at promoters and enhancers and this caused small but significant decrease in the levels of YY1 at these regions (FIG. 13). Super-enhancers are clusters of enhancers that are highly transcribed (Hnisz et al., 2015), and DRB treatment had a profound effect on transcription at these sites (FIG. 13). Similar results were observed with additional inhibitors (FIG. 13). When transcription was allowed to resume after DRB removal, the levels of YY1 increased at promoters and enhancers (FIG. 12B, FIG. 13A). These results suggest that nascent RNA produced at promoters and enhancers contributes to YY1 binding to these elements.

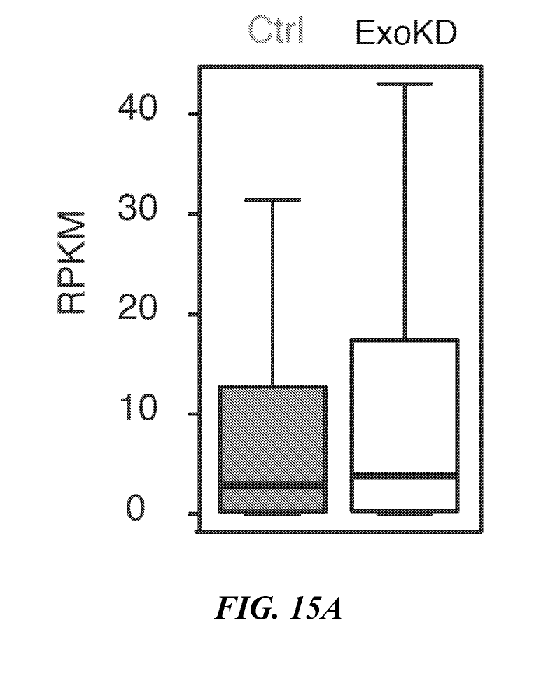

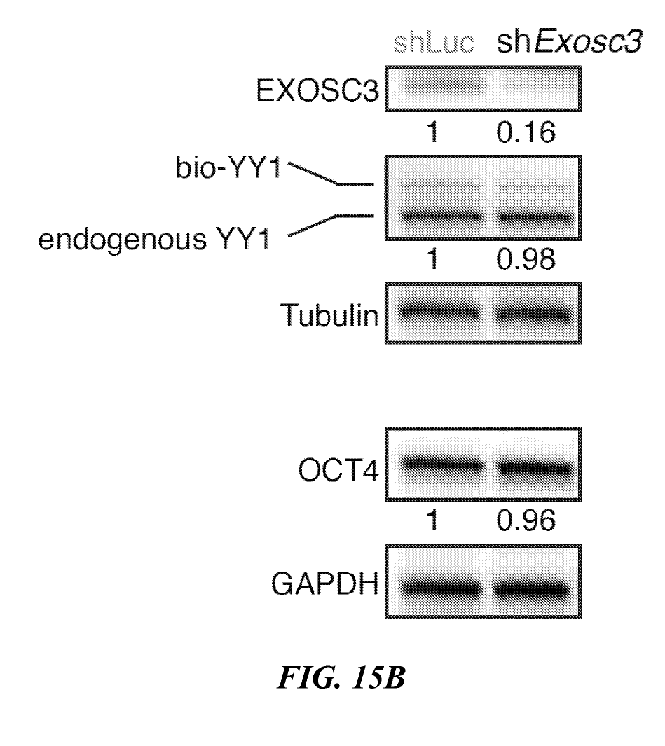

The exosome reduces the levels of enhancer RNAs once they are released from Pol II (degradation is 3′ to 5′) (Lubas et al., 2015, so knockdown of an exosome component will cause an increase in untethered enhancer RNA, which might titrate some YY1 away from enhancers. Indeed, exosome knockdown led to increased steady state levels of enhancer RNAs and a decrease in the levels of YY1 bound to enhancers (FIG. 12C, FIG. 15). These results are consistent with the model that YY1 binding to DNA is stabilized by binding to nascent RNA.

If YY1 binding to DNA is stabilized by its binding to RNA, then RNase treatment of chromatin should reduce YY1 occupancy. Chromatin was extracted from ESC nuclei and the levels of YY1 in the chromatin preparation were compared with and without RNase A treatment (FIG. 12D). The results show that the levels of YY1 bound to chromatin were significantly decreased when the chromatin preparation was treated with RNase, consistent with the idea that RNA contributes to the stability of YY1 in chromatin.

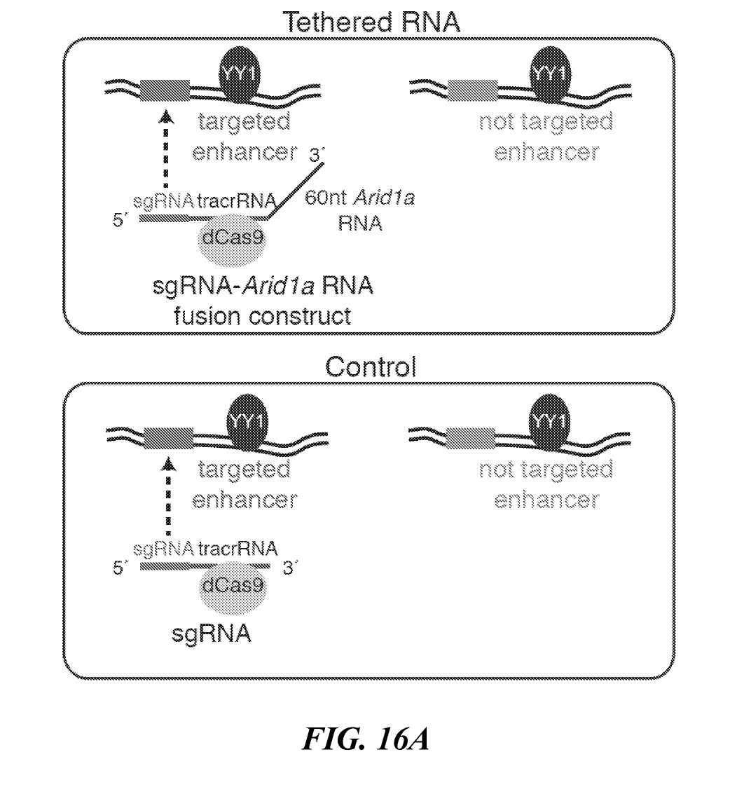

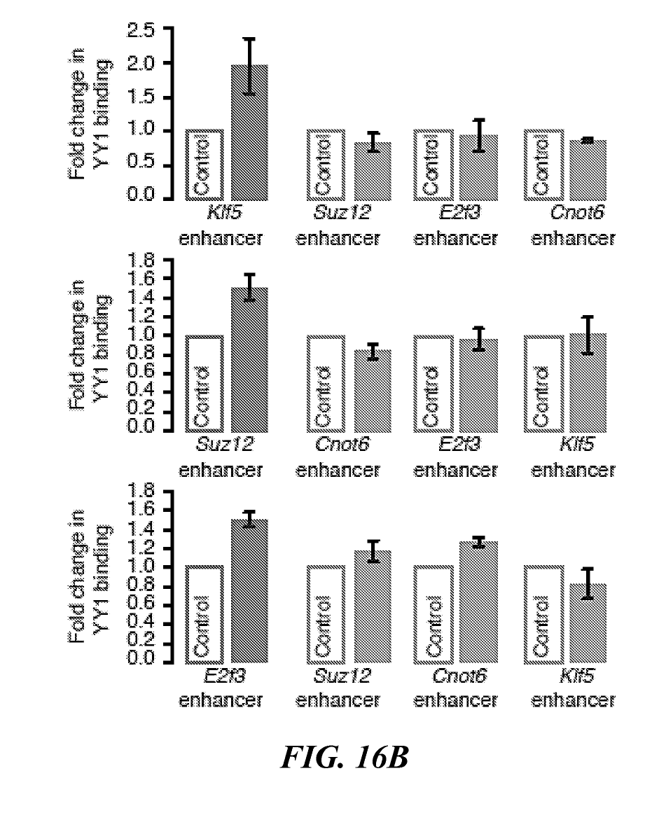

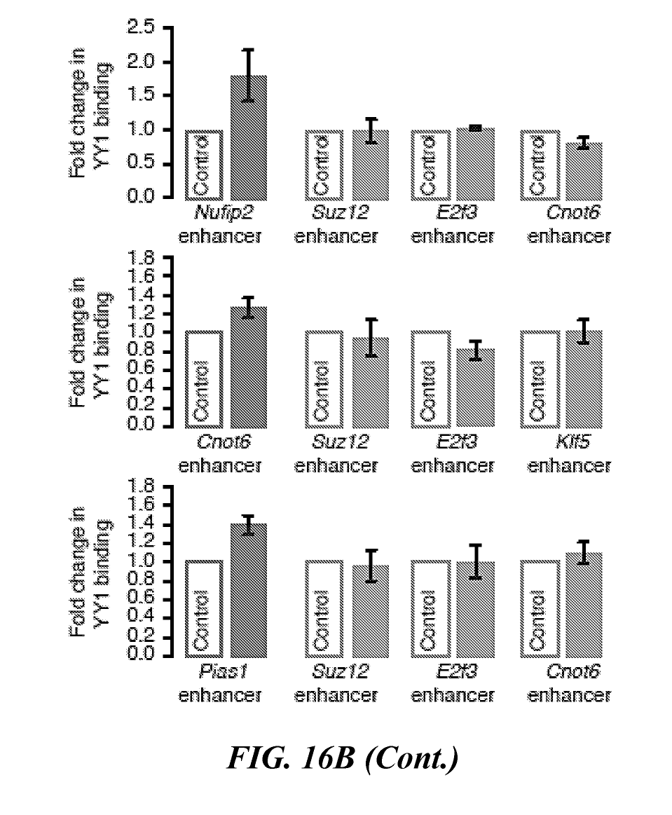

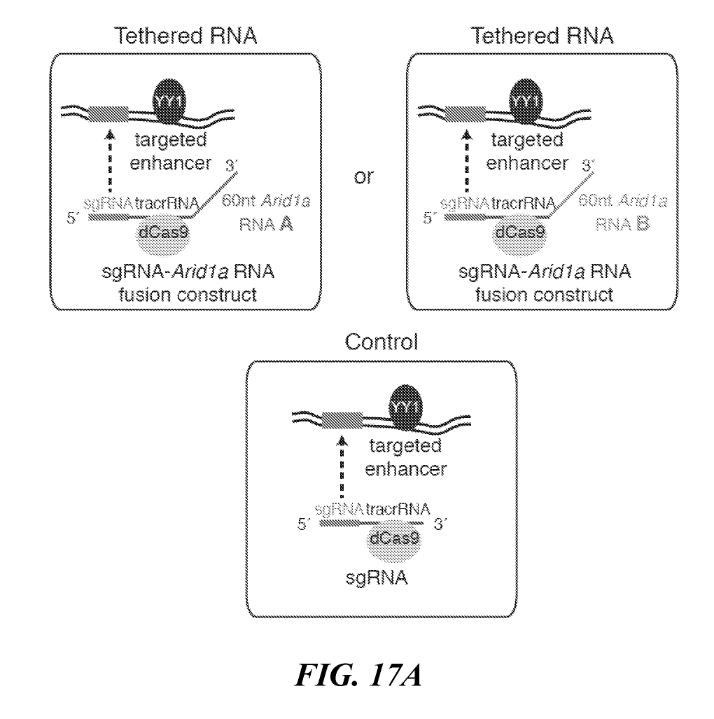

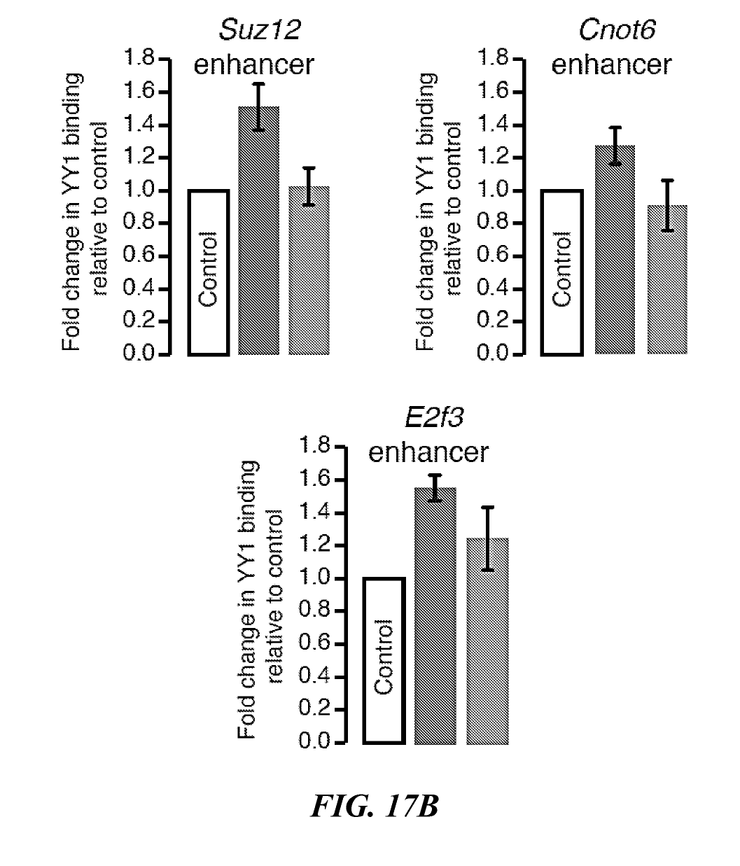

To test the idea that RNA near regulatory elements can contribute to stable TF occupancy in vivo, RNA was tethered in the vicinity of YY1 binding sites at six different enhancers in ESCs using the CRISPR/Cas9 system and it was determined whether the tethered RNA increases the occupancy of YY1 at these enhancers (FIG. 16). Stable murine ESC lines were generated expressing both the catalytically inactive form of bacterial endonuclease Cas9 (dCas9) and a fusion RNA composed of guide RNA (sgRNA), tracrRNA, and a 60-nt RNA derived from the promoter sequence of Arid1a compatible with YY1 binding in vitro (FIG. 10). For controls, stable cell lines were created that express dCas9 and sgRNA fused to tracrRNA for the six enhancers. Tethering the Arid1a RNA at each enhancer led to increased binding of YY1 to the targeted enhancer as measured by ChIP-qPCR (FIG. 16B). This elevation in YY1 binding was specific to the targeted locus and the sequence of tethered RNA as there was no observable increase in YY1 binding at the enhancers not targeted in the same cells (FIG. 16B) or targeted with tethered RNA not compatible with YY1 binding in vitro (FIG. 17). These results show that RNA tethered near regulatory elements in vivo can enhance the level of YY1 occupancy at these elements.

To corroborate the in vivo RNA tethering results, a competition EMSA was used to test whether tethered RNA increases the apparent binding affinity of YY1 to its motif in DNA (FIG. 18, FIG. 19). A short 30-bp labeled DNA probe containing a consensus YY1 binding motif was incubated with recombinant murine YY1 protein in the presence of increasing concentrations of cold competitor DNA with tethered or untethered RNA, and the amount of radiolabeled DNA that remained bound was quantified (FIG. 19). This analysis revealed that DNA containing tethered RNA outcompetes the DNA without tethered RNA for YY1 binding. These results indicate that tethering RNA near the YY1 binding motif in DNA leads to increased binding of YY1 to DNA in vitro.

### SUMMARY

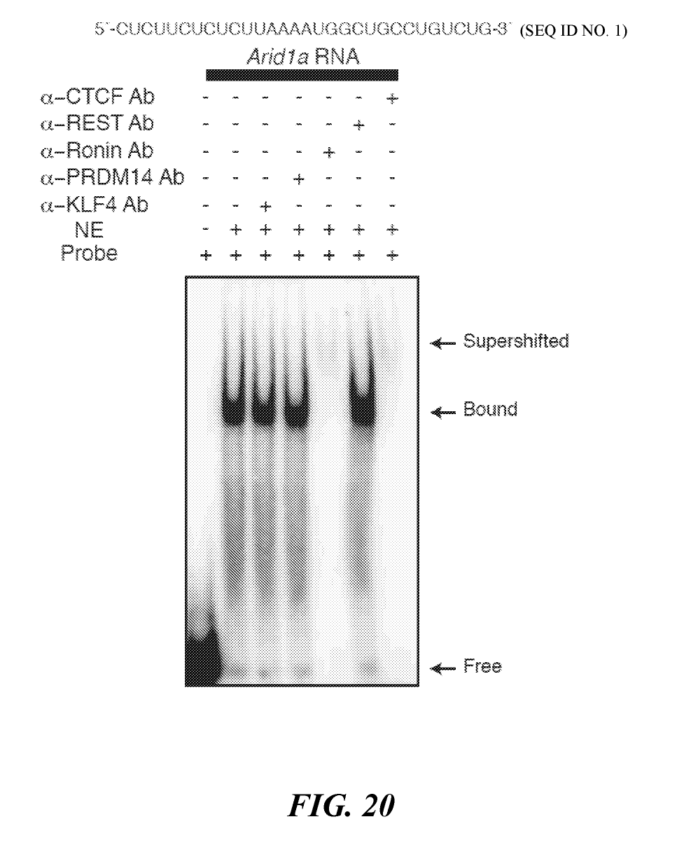

In summary, these results are consistent with the proposal that RNA enhances the level of YY1 occupancy at active enhancer and promoter-proximal regulatory elements (FIG. 12A). It is suggested that nascent RNA produced in the vicinity of enhancer and promoter elements captures dissociating YY1 via relatively weak interactions, which allows this TF to rebind to nearby DNA sequences, thus creating a kinetic sink that increases YY1 occupancy on the regulatory element. The observation that YY1 occupies active enhancers and promoters throughout the ESC genome where RNA is produced, coupled with evidence that YY1 is expressed in all mammalian cells, suggests that this model is general. There are additional DNA-binding TFs that can bind RNA (FIG. 20) (Cassiday et al., 2002), so transcriptional control may generally involve a positive feedback loop, where YY1 and other TFs stimulate local transcription, and newly transcribed nascent RNA reinforces local TF occupancy. This model helps explain why TFs occupy only the small fraction of their consensus motifs in the mammalian genome where transcription is detected and suggests that bidirectional transcription of active enhancers and promoters evolved, in part, to facilitate trapping of TFs at specific regulatory elements. The model also suggests that transcription of regulatory elements produces a positive feedback loop that contributes to the stability of gene expression programs in cells. In addition, much of disease-associated sequence variation occurs in enhancers (Hnisz et al., 2013; Maurano et al., 2012) and may thus affect both DNA and RNA sequences that interact with gene regulators.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
L. J. Core, J. J. Waterfall, J. T. Lis, Science 322, 1845-1848 (2008).
A. C. Seila et al., Science 322, 1849-1851 (2008).
A. A. Sigova et al., Proc Natl Acad Sci USA 110, 2876-2881 (2013).
T. K. Kim et al., Nature 465, 182-187 (2010).
D. Wang et al., Nature 474, 390-394 (2011).
C. A. Melo et al., Mol Cell 49, 524-535 (2013).
F. Lai et al., Nature 494, 497-501 (2013).
M. T. Lam et al., Nature 498, 511-515 (2013).
W. Li et al., Nature 498, 516-520 (2013).
M. U. Kaikkonen et al., Mol Cell 51, 310-325 (2013).
K. Mousavi et al., Mol Cell 51, 606-617 (2013).
A. Di Ruscio et al., Nature 503, 371-376 (2013).
K. Schaukowitch et al., Mol Cell 56, 29-42 (2014).
L. A. Cassiday, L. J. Maher, 3rd, Nucleic Acids Res 30, 4118-4126 (2002).
Y. Jeon, J. T. Lee, Cell 146, 119-133 (2011).
S. Gordon, G. Akopyan, H. Garban, B. Bonavida, Oncogene 25, 1125-1142 (2006).
R. A. Flynn, A. E. Almada, J. R. Zamudio, P. A. Sharp, Proc Natl Acad Sci USA 108, 10460-10465 (2011).
P. Preker et al., Science 322, 1851-1854 (2008).
H. B. Houbaviy, A. Usheva, T. Shenk, S. K. Burley, Proc Natl Acad Sci USA 93, 13577-13582 (1996).
D. Hnisz et al., Cell 155, 934-947 (2013).
M. Lubas et al., Cell Rep 10, 178-192 (2015).
M. T. Maurano et al., Science 337, 1190-1195 (2012).
W. A. Whyte et al., Cell 153, 307-319 (2013).
L. Wang et al., Nature 523, 621-625 (2015).
P. Vella, I. Barozzi, A. Cuomo, T. Bonaldi, D. Pasini, Nucleic Acids Res 40, 3403-3418 (2012).
J. Kim, J. Chu, X. Shen, J. Wang, S. H. Orkin, Cell 132, 1049-1061 (2008).
L. A. Boyer et al., Nature 441, 349-353 (2006).
R. Schmieder, R. Edwards, Bioinformatics 27, 863-864 (2011).
J. Kim, A. B. Cantor, S. H. Orkin, J. Wang, Nature Protocols 4, 506-517 (2009).
B. Langmead, C. Trapnell, M. Pop, S. L. Salzberg, Genome Biol 10, R25 (2009).
Y. Zhang et al., Genome biology 9, R137 (2008).
W. J. Kent et al., Genome research 12, 996-1006 (2002).
C. E. Grant, T. L. Bailey, W. S. Noble, Bioinformatics 27, 1017-1018 (2011).
J. C. Bryne et al., Nucleic acids research 36, D102-106 (2008).
A. Jolma et al., Cell 152, 327-339 (2013).
C. Trapnell, L. Pachter, S. L. Salzberg, Bioinformatics 25, 1105-1111 (2009).
L. Wang, S. Wang, W. Li, Bioinformatics 28, 2184-2185 (2012).
J. Loven et al., Cell 151, 476-482 (2012).
C. Y. Lin et al., Cell 151, 56-67 (2012).
A. R. Quinlan, I. M. Hall, Bioinformatics 26, 841-842 (2010).
A. A. Sigova et al., Proc Natl Acad Sci USA 110, 2876-2881 (2013).
D. Wang et al., Nature 474, 390-394 (2011).
E. Kopylova, L. Noe, H. Touzet, Bioinformatics 28, 3211-3217 (2012).
W. J. Kent, A. S. Zweig, G. Barber, A. S. Hinrichs, D. Karolchik, Bioinformatics 26, 2204-2207 (2010).
M. Jangi, P. L. Boutz, P. Paul, P. A. Sharp, Genes & development 28, 637-651 (2014).
M. Martin, EMBnet journal 17, 10-12 (2011).
B. Langmead, S. L. Salzberg, Nature Methods 9, 357-359 (2012).
H. Li et al., Bioinformatics 25, 2078-2079 (2009).
F. M. Cernilogar et al., Nature 480, 391-U151 (2011).
A. C. Mullen et al., Cell 147, 565-576 (2011).
H. Wang et al., Cell 153, 910-918 (2013).
N. A. Kearns et al., Development 141, 219-223 (2014).

Although the foregoing subject matter has been described in some detail by way of illustration and example for purposes of clarity of understanding, it will be understood by those skilled in the art that certain changes and modifications can be practiced within the scope of the appended claims.

1. A method of modulating expression of a target gene, the method comprising modulating binding between a ribonucleic acid (RNA) transcribed from at least one regulatory element of a target gene and a transcription factor which binds to both the RNA and the at least one regulatory element, wherein modulating binding between the RNA and the transcription factor modulates expression of the target gene.

2. The method of claim 1 wherein the RNA is a non-coding RNA selected from the group consisting of enhancer RNA, promoter RNA, super-enhancer constituent RNA, and combinations thereof.

3. (canceled)

4. The method of claim 1, wherein modulating binding comprises promoting binding between the RNA and the transcription factor.

5. The method of claim 4, wherein promoting binding between the RNA and the transcription factor stabilizes occupancy of the transcription factor at the at least one regulatory element, thereby increasing expression of the target gene.

6. (canceled)

7. The method of claim 1, wherein modulating binding comprises interfering with binding between the RNA and the transcription factor.

8. The method of claim 1, wherein interfering with binding between the RNA and the transcription factor destabilizes occupancy of the transcription factor at the at least one regulatory element, thereby decreasing expression of the target gene.

9.-12. (canceled)

13. The method of claim 1, wherein the transcription factor is Yin-Yang 1 (YY1).

14. (canceled)

15. The method of claim 1, wherein modulating expression of the target gene comprises contacting a cell with an effective amount of an agent which interferes with binding between the RNA and the transcription factor.

16.-20. (canceled)

21. The method of claim 15, wherein the agent comprises a decoy RNA.

22. (canceled)

23. The method of claim 21, wherein the decoy RNA comprises a synthetic RNA that comprises a nucleotide sequence that comprises an RNA binding site for the transcription factor.

24.-32. (canceled)

33. The method of claim 15, wherein the agent inhibits a component of the exosome.

34. (canceled)

35. The method of claim 1, wherein the target gene comprises a gene for which increased or aberrant transcription is associated with a disease, condition, or disorder.

36. (canceled)

37. The method of claim 1, wherein the target gene comprises an oncogene.

38. The method of claim 1, wherein the target gene comprises at least one mutation in the at least one regulatory element, wherein the at least one mutation results in the transcription factor binding to RNA transcribed from the at least one regulatory element in a manner that stabilizes occupancy of the transcription factor to the at least one regulatory element, thereby increasing expression of the target gene.

39. (canceled)

40. A method of identifying a candidate agent that interferes with binding between RNA transcribed from at least one regulatory element and a transcription factor which binds to the RNA and to the at least one regulatory element, the method comprising assessing binding between RNA transcribed from at least one regulatory element and a transcription factor which binds to the RNA and to the at least one regulatory element in the presence and absence of a test agent, wherein decreased binding of the transcription factor to the RNA transcribed from the at least one regulatory element in the presence of the test agent as compared to the absence of the test agent indicates that the test agent is a candidate agent that interferes with binding between the RNA and the transcription factor.

41.-43. (canceled)

44. The method of claim 40, wherein assessing binding comprises contacting a complex or mixture comprising the transcription factor, the at least one regulatory element, and the RNA transcribed from the at least one regulatory element with the test agent.

45.-46. (canceled)

47. The method of claim 40, wherein the test agent comprises a decoy RNA.

48. (canceled)

49. The method of claim 47, wherein the decoy RNA comprises a synthetic RNA that comprises a nucleotide sequence that comprises an RNA binding site for the transcription factor.

50.-51. (canceled)

52. The method of claim 40, wherein binding is performed in a cell.

53. The method of claim 40, wherein the method comprises performing cross-linking immunoprecipitation (CLIP) with the RNA and the transcription factor.

54.-55. (canceled)
